(19) United States
(12) Patent Application Publication
(10) Pub. No.: US 2008/0124738 A1
(43) Pub. Date: May 29, 2008

(54) COMPOSITIONS AND METHODS OF TESTING FOR TUBERCULOSIS AND MYCOBACTERIUM INFECTION

(75) Inventors: Lawrence R. Green, Tacoma, WA (US); Anne L. Sherwood, Kirkland, WA (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: PRITEST, INC, Redmond, WA (US)

(21) Appl. No.: 11/740,383

(22) Filed: Apr. 26, 2007

Related U.S. Application Data
(63) Continuation-in-part of application No. 11/069,351, filed on Mar. 1, 2005.
Application No. 11/069,351 is a continuation-in-part of application No. PCT/US05/43642, filed on Dec. 2, 2005.
(60) Provisional application No. 60/797,223, filed on May 3, 2006.
(60) Provisional application No. 60/819,199, filed on Jul. 7, 2006.

(51) Int. Cl. G01N 33/53

(52) U.S. Cl. 435/7.1

(57) ABSTRACT

The present disclosure concerns methods, compositions and apparatus for detecting pathogens and/or molecular markers. In a particular embodiment, the pathogen to be detected may be Mycobacterium bovis or any other Mycobacterium species that causes tuberculosis in a mammal. However, the disclosed methods are not limited and virtually any type of pathogen and/or molecular marker may be screened and detected. Preferred embodiments comprise reflex supplemental testing using the same assay at approximately 100% sensitivity and the highest possible corresponding sensitivity—in one example 70%. Such assay conditions, used iteratively, result in elimination of 70% of uninfected subjects for each round of testing. Use of 4 or more rounds of testing results in less than 1% error. Since only positive samples are retested, the methods provide a rapid, inexpensive and highly accurate way to detect infected subjects.

### RELATED APPLICATIONS

The present application claims the benefit under 35 U.S.C. §119(e) of Provisional U.S. Patent Application Ser. No. 60/797,223, filed May 3, 2006, and 60/819,199, filed Jul. 7, 2006. The present application is a continuation-in-part of U.S. patent application Ser. No. 11/069,351, filed Mar. 1, 2005, and of PCT/US2005/043642, filed Dec. 2, 2005. The entire contents of each priority application are incorporated herein by reference.

### FIELD

The present invention relates to methods for detecting the presence of pathogens and/or marker molecules in biological samples. In a particular embodiment, the pathogen is a Mycobacterium, such as M. bovis or M. tuberculosis. The methods involve use of a highly sensitive and selective test for the presence of a pathogen and/or marker molecule, such as antibodies against the pathogen. Because sensitivity can be selected to be at or close to 100%, a negative result is considered to be indicative of the absence of the pathogen or marker. In a more particular embodiment, samples exhibiting positive test results may be subjected to reflex supplemental testing, using the same assay, with sensitivity set at or close to 100%. A second positive result may be used as an indication for a third round of testing. With each round of reflex testing, the number of false positive results is decreased. The presence of false positives may be eliminated or reduced to any desired level by selection of an appropriate number of iterative reflex tests, depending on number of subjects tested, sensitivity and specificity of the assay used. Use of a selected number of rounds of reflex testing of positive samples may result in virtual elimination of false positives.

### BACKGROUND

Tuberculosis is a major world health issue for humans and animals, with the human disease causing approximately 2 million deaths annually (WHO 1992 Tuberculosis control and research strategies for the 1990s. Bulletin W.H.O. 70:17-21) and the animal disease posing a major cause of economic loss and a significant source of zoonotic infection (Dabom and Grange, Br. Vet. J. 1993, 149:405-17).

Bovine tuberculosis is an infectious and highly contagious disease in cattle, caused by infection with Mycobacterium bovis, a close relative of the human pathogen, Mycobacterium tuberculosis (Mtb). Very few differences have been found between the antigens expressed by these two species, both of which are included in what has been defined as “the tuberculosis complex.” However, clear variations in antigen expression differentiate these disease-causing agents from nonpathogenic strains. (Pollock and Anderson, Infect. Immun., 1997, 65:2587-92; Anderson et al., Infect. Immun. 1992, 60:2317-23; Harboe et al., Infect. Immun., 1996, 64:16-22) Bovine tuberculosis can also infect humans, other domestic animals and some wildlife including elk and whitetail deer. In the UK, badgers are heavily infected with TB and closely parallel the rates of infection for cattle.

Bovine tuberculosis may be spread by aerosol exposure to Mycobacterium or by ingestion of contaminated material. The disease tends to progress as a chronic inflammation, that is relatively asymptomatic during the early stages. Advanced cases are characterized by nonspecific symptoms such as weakness, loss of appetite, lymph node swelling, persistent cough and respiratory distress and may be mistaken for other types of respiratory disease.

A bovine tuberculosis eradication program is currently in place in the US and in many other countries. Prevalence tracking began in 1917 and decreases in prevalence related to eradication programs result in benefits observed each year. Economic benefits in 2004 were estimated to be $190,000,000 per year. Still, TB infections are frequently discovered. In fiscal year 2003, 10 affected cattle herds were detected and 6 more were detected in 2004 in the U.S. The source of infected dairy heifers/steers may be related to either undetected TB in U.S. dairies, intermingling with infected feeder cattle, or illegal movement of cattle from TB-infected areas. Inadequate animal identification may also hinder efforts to identify the source of infection.

Working to better control and eventually to eliminate tuberculosis poses a serious challenge due to the difficulty of identifying infected subjects using current tests. In most diseases, humans and animals readily develop detectable levels of antibodies in response to foreign proteins which give a reliable indication of infection with a particular pathogen. Indeed, modern disease diagnosis and surveillance has generally been based on detection of antibodies—looking for the markers of the body's response to a disease agent, rather than the disease agent itself.

However, the TB pathogen is one of a group of more insidious disease agents in which few detectable antibodies are observed by traditional methods until late in infection, when the pathogen is well established and clinical signs of disease are already apparent. It has been demonstrated that most M. bovis infected cattle mount an effective cell-mediated immune (CMI) response, have a low antibody response and contain the infection within localized foci for long periods. (Theon and Morris, 1983, Vet. Bull. Weybridge 53:543-50)

For that reason, diagnostic tests measuring specific antibodies developed in response to TB infection have proved to be less sensitive than the tuberculin PPD skin test (in both humans and cattle) as they yield many false negatives which represent missed cases of TB. The traditional serologic tests have proven to have low specificity; antibodies produced by the body in response to other comparatively harmless mycobacteria give rise to false positives. Frequently, BCG causes interference making the TB detection in BCG vaccinated subjects very difficult by either skin testing or antibody testing.

Standard methods of detection presently include tuberculin skin testing. However, false negative or false positive results of the standard skin test are not uncommon. As a positive test is likely to result in destruction of the putatively infected animal, along with other herd animals that have been directly exposed to that animal, a need exists for a more sensitive and accurate method for tuberculosis testing. Such a method would also be of benefit for testing human subjects for tuberculosis and other diseases.

Reflex testing to reduce the incidence of false positive results has been recommended for such infectious diseases as hepatitis C viral infection, human immunodeficiency virus (HIV) infection and hepatitis B infection (e.g., Morbidity and Mortality Weekly Report, Dept. Health and Human Services, Centers for Disease Control and Prevention, Feb. 7, 2003, 52(RR-3):1-13). However, such previous reflex testing has been performed using a different type of assay than the one used to generate the initial positive result. For example, the Centers for Disease Control and Prevention (CDC) has recommended that an initial positive test result for the presence of anti-hepatitis C antibody be followed up with a reflex test using a more specific serologic test, such as recombinant immunoblot assay or a nucleic acid screening test (Id.) Because such tests are often more expensive, time consuming and require a greater degree of technical expertise, reflex testing using different, more sensitive assays may considerably increase the expense, delay, or difficulty of obtaining definitive test results. Previous examples of reflex testing have focused on the use of an initial test of lower accuracy but greater speed, economy or convenience, followed by a reflex test of higher accuracy, usually with slower speed, greater expense and more complicated testing procedures. Among other things, such testing protocols are plagued by the presence of false negatives, resulting in a residual pool of infected subjects that may spread the disease. A need exists for a reflex testing method that may repetitively utilize the same type of assay to reduce or eliminate false positive and false negative results.

### SUMMARY

The present invention resolves an unfulfilled need in the art by providing a rapid (in some cases 2 hour or less), reliable, sensitive, highly specific and inexpensive test for infection with or exposure to pathogens, more particularly for the presence of antibodies against pathogens. In specific embodiments, the pathogen is a Mycobacterium, such as M. bovis or M. tuberculosis. In certain embodiments, the testing procedure may utilize a reflex supplemental testing protocol. The subjects may be mammals, such as humans, cattle, badgers, elk, deer or any other animal subject to infection with the pathogen to be detected.

The skilled artisan will realize that the reflex testing methods disclosed and claimed herein are not limited to tuberculosis in either bovines or humans, but rather may be applied to testing for the presence of a wide range of pathogens and/or marker molecules. A number of different tests for various pathogens and/or marker molecules are known in the art and commercially available. It is within the skill in the art to vary the conditions used to perform such tests to provide a higher or lower level of stringency, for example by varying parameters such as temperature, pH, number and/or stringency of wash steps, the presence, absence or concentration of agents such as detergents, chaotrophic agents (e.g., formamide, urea, guanidinium, isothiocyanate), chelating agents (e.g., EGTA, EDTA), compounds to reduce non-specific binding of antibodies or other detection moieties (e.g., bovine serum albumin), salt concentration, divalent cation concentration and other techniques known in the art. Using such techniques, the relative sensitivity and/or specificity of a given test may be adjusted. In many cases, the sensitivity and specificity vary inversely. That is, as the sensitivity of the assay is increased, the specificity is decreased due to non-specific binding or cross-reactivity with other, similar pathogens or molecules.

In preferred embodiments using the presently disclosed and claimed reflex testing methods, assay conditions may be selected to provide sensitivity at or close to 100%, with correspondingly reduced specificity. Such conditions may be selected to eliminate the presence of false negative results, allowing samples that give negative test results to be eliminated from further testing. False positives may be reduced or eliminated by iterative testing of only those samples giving positive test results in the previous testing cycle. The testing methods disclosed an claimed herein provide substantial advantages over prior art testing methods in terms of economy, speed, efficiency, simplicity of testing and reduction or elimination of false positives and false negatives.

Certain embodiments concern the use of the SeraLyte™ method for testing serum to accurately detect evidence of TB infection, which has demonstrated in test optimization as well as in blinded studies that it is possible to detect infection reliably based on antibody titers with a 2 hour assay procedure. Furthermore, it appears that the same antigen, affixed to a ferrite microbead, can be used to accurately detect TB infection in cattle, badgers, and humans. Unlike interferon based tests, such as Bovigam™, that have been used for diagnostics in recent years, antibody detection is far easier and does not require that the blood be tested within hours of its collection for accurate test results.

The SeraLyte-Mbv™ rapid diagnostic test for detecting antibodies to Mycobacterium bovis provides an ideal serological method for widespread screening of TB infection in cattle and this test is far superior to the current skin test in a number of ways:
the test appears to yield highly accurate results in about 2 hours for an initial screen;
only a small amount (as little as 0.05 ml of serum) is required from a single blood draw;
animals are not sensitized in the sample-testing process such as in skin testing;
test performance does not require a high level of technical expertise; and
test results are completely objective because they are calculated automatically by built-in software.

It is anticipated that the test will prove to be very economical to use in screening large numbers of cattle, especially since the high accuracy inherent in the test design minimizes the number of animals that will have to be retested. With reflex supplemental testing (RST, described below) the final results are obtained without having to rely on the more expensive and complex testing characterized by Bovigam. Furthermore, for the claimed assay method the blood sample can be retested at any time, unlike Bovigam which requires testing within 8-12 hours.

### BRIEF DESCRIPTION OF THE DRAWINGS

The following drawings form part of the present specification and are included to further demonstrate certain embodiments of the present invention. The embodiments may be better understood by reference to one or more of these drawings in combination with the detailed description of specific embodiments presented herein.

FIG. 1 illustrates an exemplary Reflex Supplemental Testing scheme, assuming normal distribution for infected and non-infected cattle.

FIG. 2 shows the effect of specificity and prevalence on the RST number in testing cattle for TB.

FIG. 3 shows an exemplary crib set of 11 samples and a negative control reference, using ferrite CP10-ESAT6 fusion protein conjugate with positive and negative cattle sera samples. Samples were analyzed as disclosed in Example 1.

FIG. 4 shows an exemplary ROC curve.

FIG. 5 shows an exemplary assay result for autothreshold normalized relative luminosity screening and RST test on cattle with positive M. bovis infection.

FIG. 6 shows an exemplary comparison of PPV (Positive Predicted Values) for Caudal Fold, Bovigam and the presently disclosed Reflex Supplemental Testing methods.

FIG. 7 shows cassette detail with the centered cell over the aperture for the optical detector.

FIG. 8 illustrates an exemplary protocol for TB detection, using ferrite antigen conjugates to concentrate and isolate anti-Mycobacteria antibodies.

FIG. 9 provides a summary of exemplary cattle test results for ROC graphs for MPB83 conjugates.

FIG. 10 shows an exemplary Sensitivity and Specificity ROC graph for Badger Mbv detection (N=306, blinded study)

FIG. 11 illustrates a ROC graph for MPB83 ferrite antigen SeraLyte-TB™ testing of human serum samples (N=253)

FIG. 12 shows lyophilized 3 Year Old serum sample test results for SeraLyte-Mbv™ on USDA artificially infected cattle (blinded study format N=90)

FIG. 13 shows exemplary distribution curves for negative (1) and immune incompetent (2), early (3), and robust immune competent (3) positive for Mbv cattle

FIG. 14 example showing optimal separation of Positive and Negative for Mbv cattle serum samples in a single reflex.

FIG. 15(A) shows an exemplary microtiter well cassette.

FIG. 15(B) illustrates an exemplary aluminum block crib, of use to hold and orient the microtiter well cassette shown in FIG. 15(A).

FIG. 16 shows an exemplary sample loading template for a microtiter well cassette, as illustrated in FIG. 15(A).

### DESCRIPTION OF ILLUSTRATIVE EMBODIMENTS

Additional details illustrating exemplary embodiments of the present invention are disclosed in U.S. patent application Ser. Nos. 10/425,222, filed Apr. 29, 2003; 10/373,546, filed Feb. 24, 2003; 10/373,408, filed Feb. 24, 2003; 10/373,408, filed Feb. 24, 2003 and PCT Patent Application PCT/US2004/04675, filed Feb. 17, 2004, the text of each of which is incorporated herein by reference.

### DEFINITIONS

Terms that are not otherwise defined herein are used in accordance with their plain and ordinary meaning.

As used herein, “a” or “an” may mean one or more than one of an item.

As used herein, “capture molecule” or “probe” refers to a molecule or aggregate that has binding affinity for one or more pathogens, pathogen associated molecules, and/or marker molecules. Within the scope of the present invention virtually any molecule or aggregate that has a binding affinity for some pathogen and/or marker molecule of interest may be a “probe.” “Probes” include, but are not limited to, polyclonal antibodies, monoclonal antibodies, antibody fragments, FAb fragments, humanized antibodies, single-chain antibodies, chimeric antibodies, affibodies, oligonucleotides, polynucleotides, nucleic acids, aptamers, binding proteins, receptor proteins, biotin, streptavidin, avidin and any other known ligand that can bind to at least one pathogen and/or pathogen associated molecule.

As used herein, a “marker” molecule refers to a molecule, or aggregate of molecules, whose presence, absence and/or concentration in a sample is indicative of the presence or absence of a disease, pathogenic organism or other condition. For example, the presence of particular forms of cancer in a subject may be indicated by the presence or elevated concentrations of various marker molecules, such as prostate specific antigen (PSA), CA125, mutant forms of ras or Her2-neu protein, CEA (carcinoembryonic antigen) and other molecules. Similarly, Alzheimer's disease may be indicated by the presence or elevated levels of amyloid beta peptide, amyloid precursor protein (APP) or other known markers. Many such marker molecules or molecular complexes are known in the art for different disease states or conditions and any such known marker may be assayed for using the claimed methods.

As used herein, a “pathogen” is any virus, bacterium, microorganism, molecule or molecular aggregate known in the art to be associated with an infectious disease. Non-limiting examples of pathogens are listed in Table 1.

As used herein, “quantum efficiency” means the fraction of light or photon flux that is utilized or contributes to current or signal output for an imaging device.

As used herein, “image sensor”, “imaging device”, or “imager” refers to a device for capturing an image. The term includes, but is not limited to, a CMOS (complementary metal oxide semiconductor) image sensor and a CCD (charge-coupled device) imager.

As used herein, “photon flux” means the energy of photons striking a surface, including the surface of an image sensor. The energy striking a surface may be measured in watts per cm² and correlates with the number of photons striking a unit area over a given period of time.

TABLE 1
Non-limiting Exemplary Pathogens

Actinobacillus spp.
Actinomyces spp.
Adenovirus (types 1, 2, 3, 4, 5 et 7)
Adenovirus (types 40 and 41)
Aerococcus spp.
Aeromonas hydrophila
Ancylostoma duodenale
Angiostrongylus cantonensis
Ascaris lumbricoides
Ascaris spp.
Aspergillus spp.
Bacillus anthracis
Bacillus cereus
Bacteroides spp.
Balantidium coli
Bartonella bacilliformis
Blastomyces dermatitidis
Bluetongue virus
Bordetella bronchiseptica
Bordetella pertussis
Borrelia burgdorferi
Branhamella catarrhalis
Brucella spp.
B. abortus
B. canis,
B. melitensis
B. suis
Brugia spp.
Burkholderia mallei
Burkholderia pseudomallei
Campylobacter fetus subsp. fetus
Campylobacter jejuni
C. coli
C. fetus subsp. jejuni
Candida albicans
Capnocytophaga spp.
Chlamydia psittaci
Chlamydia trachomatis
Citrobacter spp.
Clonorchis sinensis
Clostridium botulinum
Clostridium difficile
Clostridium perfringens
Clostridium tetani
Clostridium spp.
Coccidioides immitis
Colorado tick fever virus
Corynebacterium diphtheriae
Coxiella burnetii
Coxsackievirus
Creutzfeldt-Jakob agent, Kuru agent
Crimean-Congo hemorrhagic fever virus
Cryptococcus neoformans
Cryptosporidium parvum
Cytomegalovirus
Dengue virus (1, 2, 3, 4)
Diphtheroids
Eastern (Western) equine encephalitis
virus
Ebola virus
Echinococcus granulosus
Echinococcus multilocularis
Echovirus
Edwardsiella tarda
Entamoeba histolytica
Enterobacter spp.
Enterovirus 70
Epidermophyton floccosum,
Microsporum spp. Trichophyton spp.
Epstein-Barr virus
Escherichia coli, enterohemorrhagic
Escherichia coli, enteroinvasive
Escherichia coli, enteropathogenic
Escherichia coli, enterotoxigenic
Fasciola hepatica
Francisella tularensis
Fusobacterium spp.
Gemella haemolysans
Giardia lamblia
Giardia spp.
Haemophilus ducreyi
Haemophilus influenzae (group b)
Hantavirus
Hepatitis A virus
Hepatitis B virus
Hepatitis C virus
Hepatitis D virus
Hepatitis E virus
Herpes simplex virus
Herpesvirus simiae
Histoplasma capsulatum
Human coronavirus
Human immunodeficiency virus
Human papillomavirus
Human rotavirus
Human T-lymphotrophic virus
Influenza virus
Junin virus/Machupo virus
Klebsiella spp.
Kyasanur Forest disease virus
Lactobacillus spp.
Legionella pneumophila
Leishmania spp.
Leptospira interrogans
Listeria monocytogenes
Lymphocytic choriomeningitis virus
Marburg virus
Measles virus
Micrococcus spp.
Moraxella spp.
Mycobacterium spp.
Mycobacterium tuberculosis, M. bovis
Mycoplasma hominis, M. orale,
M. salivarium, M. fermentans
Mycoplasma pneumoniae
Naegleria fowleri
Necator americanus
Neisseria gonorrhoeae
Neisseria meningitidis
Neisseria spp.
Nocardia spp.
Norwalk virus
Omsk hemorrhagic fever virus
Onchocerca volvulus
Opisthorchis spp.
Parvovirus B19
Pasteurella spp.
Peptococcus spp.
Peptostreptococcus spp.
Plesiomonas shigelloides
Powassan encephalitis virus
Proteus spp.
Pseudomonas spp.
Rabies virus
Respiratory syncytial virus
Rhinovirus
Rickettsia akari
Rickettsia prowazekii, R. canada
Rickettsia rickettsii
Ross river virus/O'Nyong-Nyong virus
Rubella virus
Salmonella choleraesuis
Salmonella paratyphi
Salmonella typhi
Salmonella spp.
Schistosoma spp.
Scrapie agent
Serratia spp.
Shigella spp.
Sindbis virus
Sporothrix schenckii
St. Louis encephalitis virus
Murray Valley encephalitis virus
Staphylococcus aureus
Streptobacillus moniliformis
Streptococcus agalactiae
Streptococcus faecalis
Streptococcus pneumoniae
Streptococcus pyogenes
Streptococcus salivarius
Taenia saginata
Taenia solium
Toxocara canis, T. cati
Toxoplasma gondii
Treponema pallidum
Trichinella spp.
Trichomonas vaginalis
Trichuris trichiura
Trypanosoma brucei
Ureaplasma urealyticum
Vaccinia virus
Varicella-zoster virus
Venezuelan equine encephalitis
Vesicular stomatitis virus
Vibrio cholerae, serovar 01
Vibrio parahaemolyticus
Wuchereria bancrofti
Yellow fever virus
Yersinia enterocolitica
Yersinia pseudotuberculosis
Yersinia pesti

### Testing for Bovine Tuberculosis

In an exemplary embodiment, the disclosed reflex testing methods may be used to test for the presence of tuberculosis causing bacteria in bovine or human subjects. In cattle, tuberculosis (TB) is caused by infection with the bacterium, Mycobacterium bovis, a close relative of the human pathogen, Mycobacterium tuberculosis. Only very limited differences have been found in the antigens expressed by these strains, while clear differences in antigen expression distinguish these disease-causing agents from nonpathogenic strains. (Pollock, J. M. and P. Andersen, “Predominant recognition of the ESAT-6 protein in the first phase of interferon with Mycobacterium bovis in cattle,” Infect Immun, 1997, 65:2587-92; Andersen, A. B. et al., “Structure and function of a 40,000 MW protein antigen of Mycobacterium Tuberculosis,” Infect. Immun., 1992. 60:2317-2323; Harboe, M. T. et al., “Evidence for occurrence of the ESAT-6 protein in Mycobacterium tuberculosis and virulent Mycobacterium bovis and for its absence in Mycobacterium bovis BCG,” 1996, Infect. Immun. 64: 16-22.)

A bovine tuberculosis eradication program is currently in place in the US and in many other countries. Prevalence tracking began in 1917 and decreases in prevalence related to eradication programs have resulted. Still, infections with bovine tuberculosis are frequently discovered. In fiscal year 2003, 10 affected cattle herds were detected and 6 more were detected in 2004. Thirty-five tuberculosis cases were disclosed by slaughter surveillance in FY 2004 compared to the discovery of 39 cases in FY 2003. In Mexico the infection rate was 0.22/10,000 cattle imported in 2004 compared to 0.34/10,000 cattle in FY 2003. In Great Britain, from 1996 through October 2004 there were confirmed 1311 infected animals out of 372,284 animals tested (0.4% prevalence). Current approximate prevalence rates are 0.2% in the US, 0.4% in Mexico and 0.4% in Great Britain.

Humans contract bovine tuberculosis by: 1) inhaling air contaminated with the bacteria after an infected animal or infected person coughs or sneezes nearby; 2) drinking unpasteurized milk from an infected cow or eating raw or undercooked meat from an infected animal; or 3) handling infected meat in the dressing and processing of animal carcasses, especially if hands aren't washed carefully prior to consuming food.

However it is acquired, tuberculosis kills more people than any other disease. According to the World Health Organization (WHO), over 2 million people died in 2003 because of tuberculosis. Current estimates are that 1 in every 3 individuals in the world has latent tuberculosis, representing nearly 3 billion infected individuals. It is highly contagious, spreading rapidly akin to the common cold and can not be eradicated in the US unless it is controlled world-wide.

The symptoms of bovine tuberculosis in humans generally relate to the transmission method and are similar to those observed in tuberculosis (Mtb) infections. These symptoms include: cough, fever, night sweats, fatigue and weight loss. Patients infected with Mbov or Mtb have typically been identified using a tuberculin purified protein derivative (PPD) skin test and are treated with the same prescription drugs in either case. The drugs used to treat TB are by no means innocuous, contributing to morbidity and mortality because patients are required to endure treatment for prolonged periods for adequate therapy. It is the leading infectious killer worldwide because it can not easily be adequately treated. The prevalence of drug resistant TB is rising rapidly and is currently greater than 3%. It takes weeks to months to confirm that an individual is infected using the current best tests available.

There are no rapid diagnostic tests that will easily identify individuals who are infected and spreading the disease because the organism is difficult to isolate, slow growing, and unpredictably disseminated. The skin test requires great expertise to interpret, can not be used reliably to identify BCG vaccinated patients who could be infected or carry the organism, and fails to identify 30-40% of those individuals who are truly infected. A rapid (1 to 4 hours) and simple blood test would be of great value in finding those individuals who are infected and treating them to eradicate and or control TB.

Eradication of Tuberculosis

The control and eradication of tuberculosis poses a serious challenge due to the difficulty of identifying infected subjects. In most diseases, humans and animals readily develop detectable levels of antibodies in response to foreign proteins, which give a reliable indication of infection with a particular pathogen. However, the TB pathogen is one of a group of disease agents in which few detectable antibodies are observed by traditional methods until late in infection when the pathogen is well established and clinical signs of disease are already apparent. It has been demonstrated that most M. bovis infected cattle mount an effective cell-mediated immune (CMI) response, have a low antibody response and contain the infection within localized foci for long periods. (Theon, C. O. and Morris, J. A., The immune spectrum of Mycobacterium bovis infections in some mammalian species: a review. 1983, Vet. Bull, Weybridge, 53: 543-550.) For that reason, diagnostic tests measuring specific antibodies developed in response to TB infection have proven to be less sensitive than the tuberculin PPD skin test (in both humans and cattle), as they yield many false negatives or missed cases of TB. These serologic tests have typically also proven to have low specificity in that they frequently detect antibodies produced by the body in response to other comparatively harmless mycobacteria, giving rise to false positives.

Diagnostic Methods in Cattle

The current preferred method for diagnosing TB in cattle is the single intradermal tuberculin test (SIDT), a field test which measures the cell-mediated immune (CMI) response to M. bovis infection. Specifically, it measures the delayed type hypersensitivity reaction, a component of the CMI response, following intradermal inoculation of bovine tuberculin PPD. Results are assessed after 72 hours. (Wood, P. R. and Jones, S. L. Bovigam™ an Internationally Accredited Diagnostic test for Bovine Tuberculosis. USAHA Proceedings, 1998, 1-9.) This test has been reported to have specificity as high as 96-99% in herds with widespread infection. (Monaghan, M., et. al., The tuberculin test. 1994, Vet. Micro. 40:111-124.)

However, the moderate sensitivity of the test (72 percent) is problematic in TB eradication programs because 28% of infected animals are incorrectly identified as uninfected. They are released back into the herd unless they are obviously ill, and will spread the infection to other uninfected cattle. Also, in some geographical areas, specificity is even further reduced because of significant cross-reactivity with atypical mycobacteria. Other infected animals not in the eradication program including white tail deer, elk, badgers, etc. also re-infect cattle previously cleared, thereby contributing to and compounding problems relating to eradication.

Both sensitivity and specificity have an important impact in eradicating the disease. With declining prevalence, the number of false positive animals detected relative to true positive animals rises due to less than 100 percent specificity. The positive predictive values decline in direct proportion to prevalence, resulting in most animals incorrectly identified as infected at low prevalence. Eradicating those animals incorrectly identified as infected is economically unacceptable. Nevertheless, in spite of these shortcomings, the skin test has been the only practical test available for screening large populations (human or animal) over the past 100 years.

Most cattle with tuberculosis do not exhibit clinical signs. They do, however, pose a serious threat to other livestock and humans. Tuberculosis prevalence is thought to be low in the US and many other countries where eradication programs are currently in process, but weakness in the program persists. In many countries a standard “in series” test is used, combining two less than perfect testing modalities. (M. Thrusfield, Veterinary Epidemiology, 2nd Edition, Blackwell Science, Malden, Mass., USA. 1995) The first test is a PPD skin test called the Caudal Fold Test (CFT) which is designed to identify either a suspect (reactor) or positive. All animals testing either positive or reactor are then retested with a Short Interval Comparative Tuberculin Test (SICTT), sometimes referred to as the comparative cervical test. Sensitivity for CFT ranges from 68-95% and specificity from 96 to 98.8%. SICTT has a sensitivity of 77-95% and a specificity greater than 99%. (Francis, J. et al., “The sensitivity and specificity of various tuberculin tests using bovine PPD and other tuberculins,” Veterinary Record, 1978 103:420-425; Monaghan, M L et al., “The tuberculin test,” Veterinary Microbiology, 1994, 40:111-124.) If this test is positive, the animal is slaughtered and tissues are collected for culture and examination by histopathology. By combining the two less than perfect tests the overall sensitivity is reduced to approximately 70% but specificity is nearly 100%, sparing incorrectly identified animals from being slaughtered. However, the reduced sensitivity results in some false negative results, with corresponding presence of cryptic infected animals resulting in a continued low level rate of infection. A good example of in series testing statistical outcome for diagnostic testing in series is discussed at prevmed.vet.ohio-state.edu/ext_{—}62b.htm.

CFP10-ESAT6 Fusion Protein

A molecular analysis has been conducted of the genetic differences between virulent M bovis and the attenuated vaccine strain M. bovis BCG. Three regions of difference designated RD-1 to RD-3 were identified using subtractive genomic hybridization. RD-1 was detected in all strains of M. tuberculosis and M. bovis, but is absent in all BCG substrains. (Maheiras, G. G. et al., 1996, “Molecular analysis of genetic differences between Mycobacterium bovis BCG and virulent M. bovis,” J. Bacteriol 178:1274-1282.) The ESAT-6 gene, encoding the Early Secreted Antigen Target 6 kD protein, is found within RD-1. ESAT-6 is a major T cell antigen which has been purified from M. tuberculosis short term culture filtrates. (Sorensen, A. L. et al., 1995, “Purification and characterization of a low molecular mass T-cell antigen secreted by M tuberculosis,” Infect. Immun. 63:1710-1717; Harboe, M. et al, 1996, “Evidence for occurrence of the ESAT-6 protein in Mycobacterium tuberculosis and virulent Mycobacterium bovis and for its absence in Mycobacterium bovis BCG,” Infect. Immun. 64:16-22.) No function has yet been ascribed to ESAT-6, but it is strongly antigenic in nature, stimulates the production of gamma interferon from mice memory immune T lymphocytes and may contribute to the development of anti-tuberculosis immunity. (Andersen, et al., 1995, “Recall of long-lived immunity to Mycobacterium tuberculosis infection in mice,” J. Immunol. 154:3359-3372.)

A new gene which is cotranscribed with ESAT-6, designated lhp, has been identified. Within the lhp/esat-6 operon, a total of 13 potential genes (or open reading frames, ORFs) have been further identified, defining a novel gene family. The Mycobacterium tuberculosis lhp gene product has been identified as CFP-10, a low molecular weight 10 kD Culture Filtrate Protein, also found in short-term culture filtrates. (Berthet, F-X., et al., 1998, “A Mycobacterium tuberculosis operon encoding ESAT-6 and a novel low-molecular-mass culture filtrate protein (CFP-10),” Microbiology 144:3195-3203.) Thus, both CFP-10 and ESAT-6 are transcribed together early in M. tuberculosis or M. bovis and both encode small exported proteins. Since the two genes occur adjacent to one another in either genome, a fusion protein recombinant antigen composed of both full length proteins was designed as a capture probe for our assay.

Bovigam Tests

A new panel of tests are currently being evaluated in which killer T-cells, activated by the immune system following exposure to M. tuberculosis or M. bovis, are assayed for gamma interferon (IFN-y) production when stimulated with specific bacterial epitopes from these organisms. The bovine application of this test is called Bovigam™, primate application is Primagam™ and human application is Quantiferon™. In these assays, whole blood is taken from the patient, and incubated overnight with tuberculin (proteins extracted from M. tuberculosis or M. bovis). If the patient (human or animal) has been previously exposed to TB, then the T cells in the blood will produce IFN-y, detectable in a simple laboratory assay. The advantages of such a test is that extraction of the test blood sample does not interfere with the immune state of the subject so repeated testing is possible if necessary (the skin test requires a minimum gap of 60 days before retesting) and can be used to detect early cases of infection.

These tests can also be used reliably in HIV infected and other immuno-compromised individuals whereas skin tests cannot. Worldwide trials of well over 300,000 cattle have shown Bovigam™ to have a sensitivity ranging from 77 to 93.6% as compared to 65.6 to 84.4% for the skin test. If Bovigam™ and the skin test are used together then even higher sensitivity can be achieved.

Quantiferon Tests

Quantiferon™ has been tested in clinical trials involving over 3,000 individuals to date. In the improved version of Quantiferon™, QuantiFERON-TB GOLD test, M. tuberculosis antigens ESAT-6 or CFP-10 (gene products expressed in early in active infections of Mtb or Mbov) is are used in place of tuberculin PPD. ESAT-6 sequences have been shown to be recognized by antibodies in sera of tuberculous non-human primates (Kanaujia, G. V. et al., “Recognition of ESAT-6 Sequences by Antibodies in Sera of Tuberculous Nonhuman Primates,” Clin. and Diag. Lab. Immunol., 2004, 11(1):222-226) and cattle (Buddle, B. M., et al. Use of ESAT-6 in the interferon-gamma test for diagnosis of bovine tuberculosis following skin testing. Vet Microbiol, 2001. 80: 37-46; Buddle, B. M., et al. Use of mycobacterial peptides and recombinant proteins for the diagnosis of bovine tuberculosis in skin test positive cattle. The Veterinary Record, 2003. 615-620; Vordermeier, H. M., et al. Use of synthetic peptides derived from the antigens esat-6 and cfp-10 for differential diagnosis of bovine tuberculosis in cattle. Clin Diagn Lab Immunol, 2001. 8: 571-8) as well as humans (Arend, S. M., et al. Antigenic equivalence of human T-cell responses to Mycobacterium tuberculosis-specific RD1-encoded protein antigens ESAT-6 and culture filtrate protein 10 and to mixtures of synthetic peptides. Infect Immun, 2000. 68: 3314-21; Arend, S. M., et al. Detection of active tuberculosis infection by T cell responses to early secreted antigenic target 6-kDa protein and culture filtrate protein 10. J Infect Dis, 2000. 181: 1850-4; Berthet, F. X., et al. A Mycobacterium tuberculosis operon encoding ESAT-6 and a novel low molecular-mass culture filtrate protein (CFP-10). Microbiology, 1998. 144: 3195-203; Brock, I., et al. Performance of whole blood IFN-gamma test for tuberculosis diagnosis based on PPD or the specific antigens ESAT-6 and CFP-10. Int J Tuberc Lung Dis, 2001. 5: 462-7.)

A large number of published clinical studies have demonstrated the utility of measuring IFN-γ responses to ESAT-6 and/or CFP-10 (using methods other than Bovigam™ or Quantiferon™) for the detection of TB infection. (Dillon, D. C., et al. Molecular and immunological characterization of Mycobacterium tuberculosis CFP-10, an immunodiagnostic antigen missing in Mycobacterium bovis BCG. J Clin Microbiol, 2000. 38: 3285-90; Pathan A. A., et al. Direct ex vivo analysis of antigen-specific IFN-γ-secreting CD4 T cells in Mycobacterium tuberculosis-infected individuals: Associations with clinical disease state and effect of treatment. J Immunol, 2001. 167: 5217-25; Shams H., et al. Contribution of CD8+ T cells to gamma interferon production in human tuberculosis. Infect Immun, 2001. 69: 3497-501; Smith, S. M., et al. Human CD8(+) T cells specific for Mycobacterium tuberculosis secreted antigens in tuberculosis patients and healthy BCG-vaccinated controls in The Gambia. Infect Immun, 2000. 68: 7144-8; Vekemans J., et al. Tuberculosis contacts but not patients have higher gamma interferon responses to ESAT-6 than do community controls in The Gambia. Infect Immun, 2000. 69: 6554-7.) However, these studies have generally used testing systems that are complex to perform and unsuitable for routine diagnostic application (e.g. purified lymphocyte culture, ELISPOT). Trials in Australia have found the sensitivity and specificity of the Quantiferon™ test to be approximately 90% and 98% respectively. By comparison, the skin test has sensitivity in the order of 70% for detecting human TB infection.

Blood vs. Skin Testing

While Bovigam™, Primagam™ and Quantiferon™ require only one blood draw, and are far more accurate than skin tests and TB antibody detection tests to date, they are considerably more expensive to run, tedious to perform as they are not automated and results are not available until up to 24 hours after incubation of activated T-cell lymphocytes with M. bovis or M. tuberculosis specific antigens. Furthermore, a fresh blood sample is required for each test and must be assayed within 24 hours of collection. Thus, these tests are impractical to use for widespread screening, and are best employed as confirmatory assays of samples testing positive by a simpler initial screening method.

In Great Britain the Intradermal Comparative Cervical Tuberculin test (SICCT/skin test) is the only test used to diagnose TB in cattle. Because the Bovigam test is considered to be more sensitive but less specific than the SICCT test, the possibility of using Bovigam to eradicate infected herds was investigated. Both conventional PPD antigens and newer synthetic antigens were used in a cost-effectiveness study to determine if the IFN test would be of use. (defra.gov.uk/animalh/tb/forum/papers/tbf71.pdf) It was observed that blood testing did result in detection before skin testing became positive, but the overall benefit economically could not be justified because of the complexity of the test and overall cost per animal.

Advantages related to blood testing over skin testing include;
Reducing the length of time a herd is under movement restriction
Removing the need to visit stock twice, as required for skin tests
Reducing veterinary and labor fees by reducing the time spent on testing
Reducing the number of rounds of testing required clearing up infection due to the test's increased sensitivity
Detecting cattle infection at an earlier stage of infection
Ability to re-test in very short intervals without interference as is a problem in skin testing (60 day minimal interval)
High sensitivity ideally suited to pick up more disease when infection is spreading rapidly
Distinguish between cattle vaccinated with BCG and cattle infected with M. bovis
Analysis of results is more precise rather than a subjective assessment of a skin reaction by a trained veterinary surgeon

The skin test (SICCT or CFT) requires the mustering of animals to administer the test and again to read results 2-3 days later. It is highly recommended that this be done by a trained veterinarian as the sensitivity of the test can be as low as 20% with inexperienced technicians. It costs much more in time and money to use experienced and trained veterinarians to administer and read the test than serum-based testing.

Although there has been a long and persistent search for serological assays which can detect circulating antibody to M. bovis, none to date have demonstrated the adequate sensitivity or specificity which would make them suitable as diagnostic tests for routine use. (Wood, P. R. and Rothel, J. S. In vitro immunodiagnostic assays for bovine tuberculosis. 1994, Vet. Micro, 40: 125-135)

### Reflex Supplemental Testing—A Rapid, Efficient and Highly Accurate Method to Identify TB-Infected Subjects

The Rapid Diagnostic Test for Mycobacterium bovis (Mbv) infection provides an ideal serological method for widespread screening of TB infection in cattle. It is superior to the skin test in a number of ways, requiring a small serum sample (as little as 0.1 ml serum from a single blood draw will provide more than enough material for multiple rounds of testing). The test yields highly accurate results in less than 1.5 hours for an initial screen, and does not require a high level of technical expertise. A single blood sample is all that is required and the results are then processed in the laboratory where results may be automatically interpreted by instrumentation and software.

The test is economical to use in screening large numbers of cattle, especially since the high accuracy inherent in the test design minimizes the number of animals that have to be retested. With reflex supplemental testing (RST) the final results are obtained without having to rely on the more expensive and complex testing characterized by Bovigam. And the blood sample can be retested at any time, having none of the time dependence requirements (15 hours) required for accurate Bovigam testing.

Immune reactivity is not affected in the Rapid Diagnostic Test since there is no tuberculin injection and the results are obtained in the laboratory within a single day (including the initial screen and 2 confirmatory reflex supplemental tests on positive samples). As with Bovigam™, the Rapid Diagnostic Test is specific for M. bovis and provides a differential diagnosis between M. bovis and other mycobacterial infections such as M. paratuberculosis. Neither test reacts positively with BCG vaccinated animals. Bovigam™ requires a fresh blood sample with special handling requirements that must be tested within 15 hours. The Rapid Diagnostic Test needs only serum to test and requires no special handling so that samples may be collected and even frozen to be tested at a later date.

A typical test involves using a recombinant fusion protein capture probe of Mtb CFP-10/ESAT-6 which has been conjugated to ferrite particles coated with a carboxyl acrylic polymer. However, the skilled artisan will realize that other capture probes may alternatively be used, such as MPB83 (e.g., Wiker et al. 1998, Infect. Immun. 66:1445-52; NCBI Accession No. BAA11027; GenBank Accession Nos. BX248344, BX248333). A defined quantity of beads is dispersed in a test serum sample which has been diluted 1:50 in a dilution buffer composed of 10 mM PBS, 0.1% BSA and 0.05% ProClin. The diluted sera and beads are incubated 15 minutes at room temperature (RT). Beads are then affixed to the walls of the sample chamber using neodymium magnets and re-dispersed in wash buffer and collected twice more. In preferred embodiments, a pair of spherical magnets may be arranged in a plastic block, one on either side of a sample chamber holder (such as a microfuge tube holder). Spherical magnets have been found to provide a more intense localized magnetic field for more efficient collection of ferrite beads from solution.

A secondary biotinylated anti-bovine antibody may be used to label bovine TB antibodies that have been captured by recombinant antigen on the ferrite beads. After a 10 minute RT incubation, the beads are again affixed to the side walls of a chamber, such as a 1.5 ml Eppendorf microfuge tube, using magnets and twice more dispersed in wash buffer and recollected. The beads with biotinylated antibody affixed to bovine antibody are then incubated for 5 minutes with horseradish peroxidase streptavidin (HRP-SA) and then twice dispersing and washing the beads.

The beads are then suspended in 1:1 luminol peroxide solution and chemiluminescence is detected in a photoimager, for example, a TOAD™ (see e.g., Provisional U.S. Patent Application 60/540,720, filed Jan. 29, 2004, incorporated herein by reference) (PriTest, Redmond, Wash.). For example, an image may be obtained in a totally dark chamber for 10 minutes to accurately measure the photon image that corresponds to the level of antibody bound to beads. A software package may be used (e.g., Slider ver 3.6, see U.S. patent application Ser. No. 10/373,408, filed Feb. 24, 2003) to process up to 12 samples in a single pass. Assigned values of positive or negative are computed based on the reference negative control sera.

Each crib set currently has as many as 11 test samples in addition to the negative control. Observed signals, given as numerical values, are entered into an automated program which calculates relative luminosity (RL) of each sample by subtracting background of the crib from the observed sample value. The delta relative luminosity (ARL) for each sample is then calculated by subtracting RL of the negative control (consisting of a pooled sample of ˜10 bovine sera which have tested negative for TB) from the RL of each sample with a statistical adjustment for setting the threshold criteria that a sample must have to read positive.

A threshold, or cutoff value, for designating a sample as negative or positive for M. bovis antibodies, is determined by a preset algorithm contained within the program. Samples testing positive in the first round of screening are retested in a second round (Reflex Test #1). Samples testing positive in this second round are again retested in a confirmatory round (Reflex #2). Any samples testing negative at any point in the reflex study are confirmed as negative. Samples testing positive after Reflex #2 are confirmed positive in the assignment.

No single test will ever consistently produce the level of Sensitivity and Specificity required to effectively isolate infected animals with 100% certainty. Reflex supplemental testing involves testing deliberately at a threshold where false positives (i.e., at or near 100% sensitivity) are expected because the threshold for detecting positives has been set sufficiently low to catch 100% of positively infected animals.

By then testing in a second batch all of those animals that tested positive for infection again using the same assay, a second batch of uninfected animals will safely be released. The reflex supplemental testing process continues in steps, retesting at each interval only those animals testing positive, until only the true positive isolates are confirmed. This method of testing rapidly centers on infected animals while allowing animals that tested falsely positive to retest negative if indeed they are not infected. It is more efficient than a single screening test at accurately confirming those animals that are infected.

Calculating the RST Number

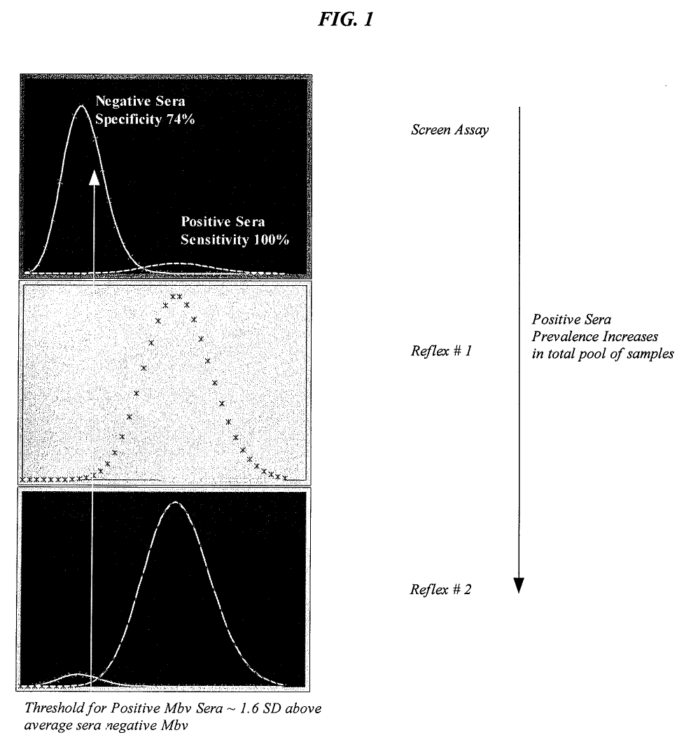

How many reflex supplemental tests are needed to with confidence stop retesting? Too many tests are of no value because time, money and resources are then wasted. This number can be easily calculated. Both seropositive and seronegative animals will have a test value that can be easily determined in an assay. A normal distribution curve of values for both the infected (positive) and uninfected (negative) animals can be determined (see FIG. 1).

The distribution curves do not need to be symmetrical (as shown) and do overlap in practice as shown in FIG. 1. If the two curves for positive and negative do not overlap, a threshold between the curves will easily differentiate between the two populations of infected and uninfected cattle with 100% specificity and sensitivity. But even if the curves overlap, as is the case with TB in cattle, a threshold may be chosen that is to the left (lower in value) of all infected (positive) animals tested. With that threshold assignment, 100% of all truly positive animals will be accurately detected (i.e., false negatives will be zero) even though some of the uninfected cattle will be falsely identified as positive as can be seen by examining FIG. 1 (i.e., a portion of the “negative sera” curve lies to the right of the threshold value). However, as can be see, with each iteration of reflex testing the number of false positive results decreases rapidly.

The assay value for each animal (either infected or uninfected) will with each test, and each reflex supplemental test, fall within one of the two curves shown in FIG. 1 and can be assigned as a positive or negative result, based on the threshold used in the assay. The value will be expected to fall anywhere under the appropriate curve (infected or uninfected) and may not necessarily with great precision reproduce the previous value measured. Variance is expected, as defined by the shape of the distribution curve.

The optimal RST number (N, equal to the number of cycles of iterative testing) depends on three factors—the total number of samples for processing (X), the number of truly infected cattle (prevalence) in a tested number of samples, and the specificity for a threshold that is sufficiently low to accurately assign all truly infected cattle a “positive” value. With each test, a percentage of the samples that are actually uninfected will be removed from the pool of samples in line for testing. Reflex supplemental testing results in each cycle enriching the percentage of positives relative to negatives that are ready for subsequent tests. This procedure is equivalent to increasing prevalence, making the positive predictive value of the test better with each re-test in the process and is shown in FIG. 1 in the change in relative distributions from Screen to Reflex # 1, to Reflex # 2, etc. The proportion of positives in the pool increases relative to the negatives in the pool with each cycle of testing. An optimized RST minimizes the false negative tests relative to the positive total tested. The total number of tests for optimization can be calculated as a sum:

### Total Number of Tests

(Screen+RSTs)=X{1+F+F²+F³+ . . . F^{N}}

### Where F is the false positive factor dependent on the specificity

F=1-Specificity
Number of false positives=F{(X−true positive #)}
X=total sample process number
N=Optimized RST Number

We can optimize with a reasonable specificity by setting the minimal criteria to eliminate 1 false positive animal assuming there is a prevalence of 1 infected animal in the total number of samples being tested. (Assuming that the specificity is substantially greater than 50%, as is the case with the exemplary protocols disclosed herein.) This is equivalent to setting the last series in the sum to a value of 1, and that allows for the calculation of N based on total number of samples (X) and specificity.

XF^{N}=1

N={Log 1−Log X}/Log F

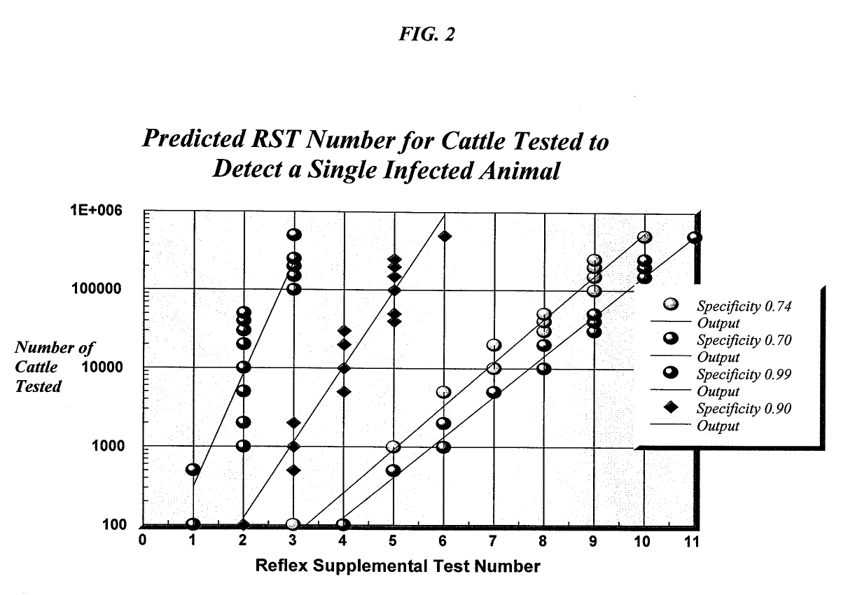

The specificity, prevalence, and number of initial tests in a screen will determine the total number of reflex supplemental tests required to with confidence assign cattle as infected or clear of infection for a given assay at 100% sensitivity. The RST number is calculated in Table 2 for 0.70, 0.74 (exemplary study), 0.90 and 0.99 specificities for 1 truly positive animal in a sample of from 100 to 500,000 cattle as shown in FIG. 2. It is apparent that the higher the specificity, the fewer total tests are required, but nevertheless the RST method quickly identifies within 3 to 4 cycles the truly infected animal in a 100 animal herd.

Optimizing the RST number reveals that a threshold about 1.6 standard deviations above the average for seronegative (uninfected) cattle will yield at 100% sensitivity a specificity of 74% in the current Rapid Mbv cattle assay. In a single screen and 3 reflex supplemental tests, the positively infected animal should be identified to within 1 of 4 animals in a herd of 1000 cattle, and could be positively identified by an additional 4 tests. Under these conditions, the total number of tests performed to positively identify the single infected animal in a 1000 animal

TABLE 2
Predicted RST Number Dependence on Specificity and Prevalence
Reflex Supplemental Testing - Number Calculator

| | | | | | | | | | | | | | |
|---|---|---|---|---|---|---|---|---|---|---|---|---|---|
| | | | | | | | | | | | | Total | Total |
| Sample # | N | R1 | R2 | R3 | R4 | R5 | R6 | R7 | R8 | R9 | R10 | Tests | Tests |

| | |
|---|---|
| Specificity | 0.99 |
| Number Positive | 1 |
| Number False Positive | 1 |
| Specificity Factor (1-Sp) | 0.01 |

| | | | | | | | | | | | | | |
|---|---|---|---|---|---|---|---|---|---|---|---|---|---|
| 100 | 1 | 1 | 0 | 0 | 0 | 0 | 0 | 0 | 0 | 0 | 0 | 1 | 101 |
| 500 | 1 | 5 | 0 | 0 | 0 | 0 | 0 | 0 | 0 | 0 | 0 | 5 | 505 |
| 1000 | 2 | 10 | 0 | 0 | 0 | 0 | 0 | 0 | 0 | 0 | 0 | 10 | 1010 |
| 2000 | 2 | 20 | 0 | 0 | 0 | 0 | 0 | 0 | 0 | 0 | 0 | 20 | 2020 |
| 5000 | 2 | 50 | 0 | 0 | 0 | 0 | 0 | 0 | 0 | 0 | 0 | 50 | 5050 |
| 10000 | 2 | 100 | 0 | 0 | 0 | 0 | 0 | 0 | 0 | 0 | 0 | 100 | 10100 |
| 100000 | 3 | 1000 | 10 | 0 | 0 | 0 | 0 | 0 | 0 | 0 | 0 | 1010 | 101010 |
| 500000 | 3 | 5000 | 50 | 0 | 0 | 0 | 0 | 0 | 0 | 0 | 0 | 5050 | 505050 |

| | |
|---|---|
| Specificity | 0.74 |
| Specificity Factor (1-Sp) | 0.26 |

| | | | | | | | | | | | | | |
|---|---|---|---|---|---|---|---|---|---|---|---|---|---|
| 100 | 3 | 26 | 6 | 1 | 0 | 0 | 0 | 0 | 0 | 0 | 0 | 34 | 134 |
| 500 | 5 | 130 | 33 | 8 | 2 | 0 | 0 | 0 | 0 | 0 | 0 | 174 | 674 |
| 1000 | 5 | 260 | 67 | 17 | 4 | 0 | 0 | 0 | 0 | 0 | 0 | 348 | 1348 |
| 2000 | 6 | 520 | 135 | 35 | 9 | 2 | 0 | 0 | 0 | 0 | 0 | 700 | 2700 |
| 5000 | 6 | 1300 | 338 | 88 | 22 | 6 | 1 | 0 | 0 | 0 | 0 | 1754 | 6754 |
| 10000 | 7 | 2600 | 676 | 175 | 45 | 12 | 3 | 0 | 0 | 0 | 0 | 3510 | 13510 |
| 100000 | 9 | 26000 | 6760 | 1757 | 457 | 118 | 31 | 8 | 2 | 0 | 0 | 35132 | 135132 |
| 500000 | 10 | 130000 | 33800 | 8788 | 2285 | 594 | 154 | 40 | 10 | 2 | 0 | 175672 | 675672 |

| | |
|---|---|
| Specificity | 0.70 |
| Specificity Factor (1-Sp) | 0.30 |

| | | | | | | | | | | | | | |
|---|---|---|---|---|---|---|---|---|---|---|---|---|---|
| 100 | 4 | 30 | 9 | 2 | 0 | 0 | 0 | 0 | 0 | 0 | 0 | 41 | 141 |
| 500 | 5 | 150 | 45 | 13 | 4 | 0 | 0 | 0 | 0 | 0 | 0 | 211 | 711 |
| 1000 | 6 | 300 | 90 | 27 | 8 | 2 | 0 | 0 | 0 | 0 | 0 | 426 | 1426 |
| 2000 | 6 | 600 | 180 | 54 | 16 | 4 | 1 | 0 | 0 | 0 | 0 | 854 | 2854 |
| 5000 | 7 | 1500 | 450 | 135 | 40 | 12 | 3 | 0 | 0 | 0 | 0 | 2139 | 7139 |
| 10000 | 8 | 3000 | 900 | 270 | 81 | 24 | 7 | 2 | 0 | 0 | 0 | 4282 | 14282 |
| 100000 | 10 | 30000 | 9000 | 2700 | 810 | 243 | 72 | 21 | 6 | 2 | 0 | 42853 | 142853 |
| 500000 | 11 | 150000 | 45000 | 13500 | 4050 | 1215 | 364 | 109 | 32 | 9 | 3 | 214280 | 714280 |

herd is 1348. Thus, it can be seen that even with multiple iterative cycles of reflex testing, the total number of tests required is relatively low compared to the herd size.

### Apparatus for Pathogen or Marker Detection

Certain embodiments of the present invention concern apparatus of use for pathogen and/or marker molecule detection assays. Additional details illustrating exemplary embodiments of the present invention are disclosed in U.S. patent application Ser. No. 09/974,089, filed Oct. 10, 2001; 10/035,367, filed Oct. 9, 2002; 10/425,222, filed Apr. 29, 2003; 10/373,546, filed Feb. 24, 2003; 10/373,408, filed Feb. 24, 2003; and provisional patent application 60/540,720, filed Jan. 29, 2004, the text of each of which is incorporated herein by reference in its entirety.

### Total Optical Assay Device (TOAD)

A non-limiting exemplary embodiment of a Total Optical Assay Device (TOAD™) comprises a light-tight casing with a hinged lid. When the lid is closed, the casing blocks external light from the interior of the TOAD™, allowing highly sensitive detection of bioluminescence or other emitted light from samples to be analyzed. The lid is opened to show a stage. Devices to be imaged, including but not limited to GRABBER™ slides (see below) or microtiter well devices, may be positioned on top of the stage. Underneath the stage is a single focusing lens that sits on top of a CCD (charge coupled device) chip or other light sensor. The slide or microtiter wells may be positioned on the stage by aligning them with corresponding shaped depressions on the top of the stage

In a non-limiting example, the imaging device 1350 is a QICAM 10-bit mono cooled CCD camera (Qimaging™, Burnaby, B.C., Canada, catalog #QIC-M-10-C). However, the TOAD™ is not limited to the exemplary embodiment and it is contemplated that other known types of imaging devices may be utilized. A single focusing lens may be interposed between the CCD chip and the stage and may be attached to the top of the imaging device, for example using a C-mount adaptor.

Cooling System

In an exemplary embodiment the imaging device contains, in addition to a CCD chip, a Pelletier heat exchange element surrounding the CCD chip. The rest of the imaging device contains electronic components for processing the signals detected by the CCD chip, along with connections for a power supply, software interface and external data port, such as a 6-pin firewire connector. The Pelletier heat exchanger removes heat from the CCD camera and releases it inside the casing. In order to prevent the TOAD™ from overheating, a separate fan driven cooling device may be positioned adjacent to the top of the imaging device. The cooling device drives hot air out of the casing through a light-tight vent at the back of the casing and circulates cool air through the casing interior. Alternatively, the Pelletier device may be attached to one or more heat dissipation devices, such as fins, heat sinks or other known devices for radiating heat. During standard operation, the cooling device operates efficiently enough that the CCD camera may be operated continuously without overheating. Overheating of the CCD camera results in substantial increase in background noise, producing random bursts of apparent light detection throughout the optical field of the CCD chip.

In preferred embodiments, modifications to the cooling system have resulted in less variability in the baseline for normal controls and better differentiation of positive from negative samples. Cooling improvements are accomplished by adding to the Peltier system a series of heat sinks and a 12 volt circulating fan. Power for the fan may be obtained by taking advantage of the second fire wire connector on the camera that is daisy chained to the main power off the computer. This reduces the number of power connectors needed to run the instrument and makes certain that the fan is always running to cool the camera when it is turned on. In some embodiments, the CCD camera is a 12 bit model 32-0090B (Q Imaging) camera with a Peltier cooling system. The housing of this system is aluminum and is located at the chip end of the camera, where the lens and bezel attach. During normal operation, this aluminum housing heats up to approximately 60° C. and the performance of the chip diminishes because the pixel black noise increases with temperature. The net effect of heating is increased noise relative to signal, making it more difficult to differentiate borderline positive samples from true negative samples in an assay.

To dissipate heat rapidly, especially in the enclosed instrument housing, we affix first to the aluminum lens bezel, a 4 inch square heat sink (anodized black aluminum with a central ±4 inch hole) to allow light to strike the lens and chip. We also affix to one side of the Peltier cooler a 4 inch square heat sink, and a fan and 4 inch square heat sink to the opposite flat surface of the cooling system. This arrangement draws air from beneath the camera housing through the instrument housing that is then exported through a light proof exit port, so that the average temperature at the chip surface housing is reduced by approximately 30° C. to slightly above room temperature. This temperature is sustained even during continuous operation for extended periods with a noticeable reduction in background noise.

The imaging device rests on top of a base that holds the imaging device in position relative to the stage. The bottom of the base may be open to allow access to on on-off switch at the bottom of the imaging device and connections to power supplies (e.g. 110 volt AC electrical cord), firewire and any other external connections.

Stage

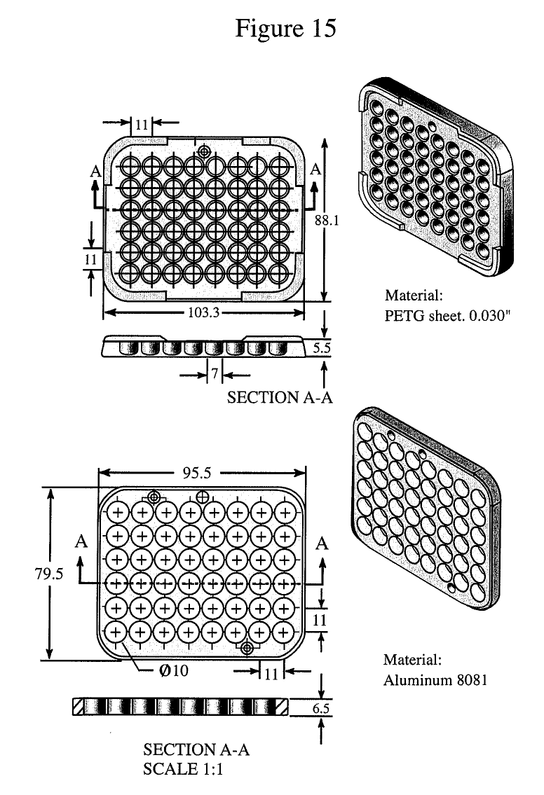

Indentations in the top of the stage may be arranged to allow positioning of slides, microtiter well plates or other devices over the CCD chip. For example, in certain embodiments an aluminum block (FIG. 15B) at the top of the stage may be machined to contain holes that align precisely with microtiter wells in the bottom of standard or custom microtiter plates. In preferred embodiments, an alignment depression on the stage matches a corresponding protrusion on the bottom of a microtiter plate so that there is only one way of inserting the plate into the stage so the plate may be correctly aligned. One or more holes in the stage allow for light transmission through the slide, microtiter well or other device and detection by the underlying CCD chip. It will be apparent to the skilled artisan that the use of devices with a transparent or translucent bottom surface in contact with the stage, as discussed in more detail below, facilitates the use of a TOAD™ apparatus with an optical detector located below the device to be optically read.

In the case of the microtiter well cassette (discussed below), the cassette is securely located on the stage in the proper orientation with an aluminum machined crib (FIG. 15B). The machined crib has 48 vertical cylinders that have centers each in perfect alignment with the template for the cassette. The crib and cassette have in addition to the 48 sample centers, a single locking point so that the cassette will only fit the crib in one way. This assures the operator that the crib locations after loading always appear in the same image position when the camera has completed its analysis of a test run. There are 12 samples for each transfer bar used in an analysis designated as A, B, C and D. The first two samples are used as the negative control set for the cassette analysis. Cassettes are loaded in the order A1 to A12, and then B1 to B12, etc. until all samples are loaded by transferring a luminol peroxide solution with ferrite particles to each well corresponding to a sample position and number ID.

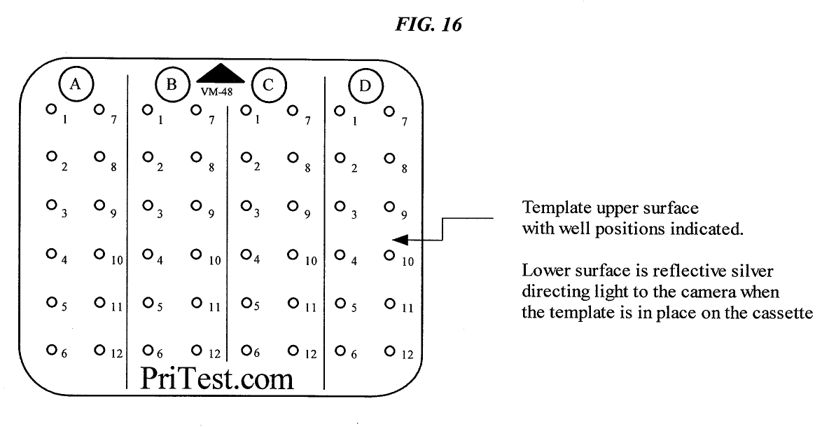

The crib is secured and calibrated on each instrument before it is released so that the well number 1 and 2 are imaged exactly as the template appears (see, e.g., FIG. 16). The sequence and number pattern for loading and interpreting the cassette is thus assured in each test run so that the operator may correlate test runs with sample ID numbers.

The aluminum crib surrounds each well, preventing light from one well cross contaminating the readings for any of its neighbors, and this is also assured when the vacuum molded cassette well sits above the cylinder by silvering of the underside of the cassette. There are two mounting bolts that affix the crib to the instrument housing.

Cassette

In certain embodiments, a vacuum molded cassette comprising microtiter wells (FIG. 15A) may be used. Advantageously, the cassette is leakproof, as it is formed of a single piece of plastic and there is no bonding between different materials to leak. It can be efficiently produced at a cost savings of more then 3× compared to the honeycomb cassette discussed below. It can be silvered to increase reflectivity, directing light generated in a well to the camera, and it is easy to load with samples. In an exemplary embodiment, the translucent vacuum molded cassette has 48 wells that hold approximately 400 μL of fluid when filled to the top. Under normal use, only 100 μL is used to obtain an image for the cell.

Cassettes are treated to produce a silver mirror surface by inverting them and filling with a silvering agent so that only the bottom of each well remains free of silver. As it is translucent, when the cassette is filled with sample solutions and a template is placed on top to fit the cassette (silver reflective surface facing the well contents), all light from the reaction is directed out the bottom through the non-silvered surface directly to the camera. The intensity of the signal is quantified relative to a negative control signal that is also measured in every cassette.

To produce cassette silvering, a silver nitrate solution is applied as described below.
Silver nitrate: 33.3 gm per liter distilled water, store in amber glass (RT)
Concentrated ammonium hydroxide (RT)
Potassium hydroxide, 0.7 M: 40 gm per liter distilled water, store in plastic (RT)
Sugar reducer: 40 gm cane sugar dissolved in 100 ml ethanol and 400 ml distilled water and 1 ml nitric acid. Boil the sugar solution 30 minutes. Store in glass or plastic container (RT).

| | For 1 | | For 10 | | For 20 | |
|---|---|---|---|---|---|---|
| Reagent | cassette | | cassettes | | cassettes | |
| silver nitrate | 18.0 | ml | 180 | ml | 360 | ml |
| conc. ammonium hydroxide | ≧0.5 | ml | ≧5.3 | ml | ≧10.5 | ml |
| potassium hydroxide | 9.0 | ml | 90 | ml | 180 | ml |
| conc. ammonium hydroxide | ≧0.5 | ml | ≧5 | ml | ≧10 | ml |

1. Add silver nitrate solution to the mixing vessel.
2. Add conc. ammonium hydroxide. Mix vigorously. The solution will turn cloudy and then it will clear. Add enough ammonium hydroxide to just clear the solution.
3. Add potassium hydroxide.
4. Add conc. ammonium hydroxide to again just clear the solution. Mix vigorously. The solution will turn cloudy and then it will clear. Add enough ammonium hydroxide to clear the solution
5. Lay the appropriate number of cassettes face down on a protected flat surface near a sink.
6. Using a 50 ml syringe, add 28 ml of silver nitrate complex to all cassettes.
7. Draw a pipette tip between the wells of the cassette to dislodge bubbles.
8. Add 1.06 ml of sugar reducer to a single cassette, distributing the solution over the surface of the silver nitrate complex with a sweeping motion. Drops of sugar reducer may land on the bottom of the cassette wells where there is no silver nitrate complex with no ill effect.
9. Immediately after the addition of the reducer, shake the cassette with a gentle vibration motion to mix the sugar reducer into the silver nitrate complex. The mixture turns black.
10. Repeat for each cassette.
11. Allow the silver to react with the plastic cassette for 10 to 15 minutes. The mixture turns tan and silver crystals will form at the liquid-air interface.
12. Remove the depleted silver solution and rinse the cassette with tap water to remove any sediment.
13. Set the cassettes face down to dry on a clean dry surface.

A template with a silvered reflective back and the markings as shown in FIG. 16, identifying the well positions is placed over the cassette when it is put on the TOAD's stage for imaging. The template is discarded after each use along with the disposable silvered cassette.

| Coordinates for Each cell | | Row A, B, C, D, E, F |
|---|---|---|
| Position | | Number indicated (left to right) |
| Horizontal Dimension | 3.6 | inches begin measurement from left to |
| | | right (indicated by “h”) |
| Vertical Dimension | 2.6 | inches begin measurement from top |
| | | down (indicated by “v”) |
| Cell Width | 0.315 | |
| Cell Height | 0.315 | |
| CC Horizontal | 0.433 | |
| CC Vertical | 0.433 | |

| | Position | | | | | | | | |
|---|---|---|---|---|---|---|---|---|---|
| Row | 1 | 2 | 3 | 4 | 5 | 6 | 7 | 8 | L to R |
| Ah | 0.433 | 0.866 | 1.299 | 1.732 | 2.165 | 2.598 | 3.031 | 3.464 | 3.031 |
| Av | 0.433 | 0.433 | 0.433 | 0.433 | 0.433 | 0.433 | 0.433 | 0.433 | |
| Bh | 0.433 | 0.866 | 1.299 | 1.732 | 2.165 | 2.598 | 3.031 | 3.464 | |
| Bv | 0.866 | 0.866 | 0.866 | 0.866 | 0.866 | 0.866 | 0.866 | 0.866 | |
| Ch | 0.433 | 0.866 | 1.299 | 1.732 | 2.165 | 2.598 | 3.031 | 3.464 | |
| Cv | 1.299 | 1.299 | 1.299 | 1.299 | 1.299 | 1.299 | 1.299 | 1.299 | |
| Dh | 0.433 | 0.866 | 1.299 | 1.732 | 2.165 | 2.598 | 3.031 | 3.464 | |
| Dv | 1.732 | 1.732 | 1.732 | 1.732 | 1.732 | 1.732 | 1.732 | 1.732 | |
| Eh | 0.433 | 0.866 | 1.299 | 1.732 | 2.165 | 2.598 | 3.031 | 3.464 | |
| Ev | 2.165 | 2.165 | 2.165 | 2.165 | 2.165 | 2.165 | 2.165 | 2.165 | |
| Fh | 0.433 | 0.866 | 1.299 | 1.732 | 2.165 | 2.598 | 3.031 | 3.464 | |
| Fv | 2.598 | 2.598 | 2.598 | 2.598 | 2.598 | 2.598 | 2.598 | 2.598 | |
| T to | 2.165 | | | | | | | | |
| B | | | | | | | | | |

### Slides

In certain embodiments, optical detection may be used in combination with chips, matrix arrays and/or slides, such as microscope slides, as a binding surface for detection of pathogens and/or marker molecules. A variety of slide substrates are known, as discussed below.

Traditional Translucent Slides

Glass or plastic microscope slides have commonly been used as solid matrix supports for microarray analysis. Probe molecules have been attached to glass or plastic surfaces using cross-linking compounds. (See, e.g., Schena, Microarray Analysis, J. Wiley & Sons, New York, N.Y., 648 pp., 2002.) Probes may be printed as 2D arrays of spots. Many different kinds of cross-linkers are known, depending on the type of reactive moieties (e.g., sulfhydryl, amino, carboxyl, phenyl, hydroxyl, aldehyde, etc.) available on the probe molecules that can be cross-linked to the surface without affecting probe functionality (e.g., target molecule binding).

A problem with previous methods for probe attachment is that the capacity for attachment is limited. As probe size is increased, the number of possible binding sites for prospective target molecules is generally decreased. If the binding sites for the probe are saturated at a level below the threshold for detection, a signal will not be observed even if binding has occurred between probe and target molecule.

Attempts have been made to attach probes to glass surfaces using avidin-coated slides and biotin-conjugated probe molecules. Alternatively, silanes, such as aminosilane or 3-glycidoxypropyltrimethoxysilane, have been coated onto the glass surface, with the silane moiety attached to the glass and the reactive moiety cross-linked to probe molecules. Other approaches have utilized slides coated with reactive substrates with functional aldehyde, carboxyl, epoxy, or amine groups that can form a covalent bond with the probe molecules, affixing them permanently to the glass surface.

Although these methods work moderately well for small probe molecules, they tend to work poorly for larger probe molecules (e.g., antibodies) where functionality (binding) may depend on probe orientation, flexibility and degree of cross-linking. Covalent attachment methods also tend to bind very little material to the matrix surface. Consequently, probe concentration is low and signal detection is difficult. Because relatively little probe is available on the surface of the 2D array, such systems show a low signal-to-noise ratio for a positive binding reaction between probe and target.

Protein or peptide target molecules are often detected using antibodies as capture molecules. Two-dimensional arrays used in clinical diagnostics or proteomics frequently utilize antibodies as probes for protein or peptide target molecules. Although antibodies tend to be highly specific for their target antigens, they are not easily attached to glass or plastic surfaces with cross-linking agents and standard methods. This is because of the limited amount of material that can be affixed to the matrix with known chemistries, resulting in weak signals generated upon target binding. Another problem is that antibody specific binding cannot be maintained without adequate hydration and support in the matrix. Thus, long-term stability of antibody-coupled solid matrix arrays tends to be limited, with inconsistent results obtained depending on the age of the array.

Attempts have been made to solve this problem by creating an environment that stabilizes the protein and preserves its functional probe features. For example, Prolinx Inc. (Bothell, Wash.) has developed a chemical affinity system using standard glass slides with a polymer brush format affixed to their surface. The system relies upon the interaction between two synthetic small molecules that form a stable complex, phenyldiboronic acid (PDBA) and salicylhydroxamic acid (SHA). (E.g., Stolowitz et al., Bioconjugate Chem. 12:229-239, 2001.) PDBA is first conjugated with protein probes. The conjugated probes then link to SHA attached to the polymer brush to form a 3D functional array. This method is limited by the amount of antibody that can be bound to the surface. More importantly, the target antigen must be sufficiently small to diffuse through the brush border in order to react with antibodies affixed to the matrix. Such methods are not suitable for identifying and/or quantifying larger targets, such as whole cells or bacteria.

Opaque Slides

Methods to stabilize and increase the amount of probe attached to matrix arrays are highly desirable. Such methods generally lead to opaque slides, since the matrix materials used to increase probe binding and preserve stability typically involve non-translucent gels, hydro-gels, agars, and other materials coated on the glass surface. Proteins attached to such opaque matrix materials are stabilized by hydrophobic and electrostatic interactions in a three-dimensional array.

Most scanners in current use for genomic and proteomic microarrays read the slides from the same side as the bound probe and target molecules, using opaque matrix arrays. Opaque matrix-coating materials used to produce microarrays include nylon, PVDF (polyvinylidene fluoride) and nitrocellulose. Nitrocellulose, a traditional polymer substrate in use for more than 50 years, is a substrate with very attractive properties for microarray applications. (E.g., Tonkinson and Stillman, Frontiers in Bioscience 7:c1-12, 2002.)

Opaque nitrocellulose has been extensively used to immobilize proteins and nucleic acids for biomolecular analysis. Nitrocellulose immobilizes molecules of interest in near quantitative fashion and allows for short and long term storage. Nitrocellulose also allows for solution phase target species to efficiently bind to immobilized capture molecules. Diagnostic devices using ELISA methods have employed nitrocellulose membranes with a lateral flow process to bind capture reagents to the membrane (Jones, IVD Technology, 5(2):32, 1999).

Traditional opaque membrane materials have a number of attractive features. They are inexpensive to construct, bind more than 100 times the amount of protein that can be bound by linker coated glass slides, and are generally easy to work with. This is particularly true for opaque nitrocellulose membranes, which have a long history of use.

Nitrocellulose is normally produced in a microporous form that may be applied to the surface of glass slides to form an opaque surface. Probes may then be attached to the opaque nitrocellulose membranes in microarrays, using standard nitrocellulose binding methods. Such slides have been used with radioactive, fluorescent and chemiluminescent detection systems (e.g., Brush, The Scientist 14[9]:21, 2000).

Traditional nitrocellulose membranes are also very brittle in the absence of a supporting structure or foundation, leading to frequent cracking or fragmentation. For this reason, opaque nitrocellulose has been used in a microporous form bound to plastic sheets. Such sheets are always opaque, due to the microporous form, and require a supporting structure (e.g. acetate or cellulose) to avoid damage during handling.

Translucent Nitrocellulose Slides

The methods and compositions disclosed herein may be used to produce translucent surface coatings of colloidal nitrocellulose that retain the advantageous binding characteristics of opaque nitrocellulose membranes, while allowing for use of detectors arranged on the bottom (non-binding) side of the slide. The interaction between probe and target molecules may be observed directly on a translucent nitrocellulose solid matrix.

In some embodiments, translucent nitrocellulose matrix arrays may be used. In such embodiments, the translucent nitrocellulose matrix may be attached to one side of a glass or plastic slide (e.g., nylon). Probes may be attached to the nitrocellulose and the interaction between probe and target molecules observed through the slide with a sensor or camera.

The nitrocellulose material is totally translucent (i.e., transparent) if formed according to the disclosed methods. Light signals may thus be observed without scatter or interference from opaque materials. This allows a greater signal-to-noise ratio and ease of detection of target molecules, compared to opaque microporous nitrocellulose matrix arrays. Such opaque matrix arrays can obscure portions of the light or reaction indicator species (e.g., bioluminescent compound) produced upon binding of target molecules.

Nitrocellulose in the form of a colloid in an amyl acetate solvent has been used by electron microscopists to make castings for specimens. Colloidal nitrocellulose is formed by casting as an ultra-thin film on a water surface. The film may then be picked up on a transmission electron microscopy (TEM) grid and used as a support film for TEM specimens. Because the film must be very clean and uniform, great care is exercised in its production. Colloidal nitrocellulose is readily soluble in amyl acetate. The amyl acetate is water soluble and evaporates evenly to form uniform films. It is supplied as a 1% solution of very pure nitrocellulose.

High purity nitrocellulose in EM grade amyl acetate (Collodion) may be purchased from commercial sources. The amyl acetate is purified by refluxing over calcium oxide to remove all moisture. Soluble and suspended material is removed by slow distillation. The removal of all traces of moisture from the solvent permits the formation of very strong colloidal nitrocellulose films with virtually no holes.

In an exemplary embodiment, Collodion was obtained in bulk from Ernest F Fullam, Inc. (Latham, N.Y.) and used to manufacture high quality translucent nitrocellulose matrix arrays. An aliquot of 200 μl of 1% Collodion solution was pipetted onto the surface of freshly cleaned standard 25×75 mm glass slides. The Collodion was evenly spread to the edges of the glass slide surface in a dust free area. After drying for 2 hours at room temperature, the slides were heated for an additional hour or more at approximately 60° C. Dried slides were labeled and stored for production of microarrays.

When using a glass array surface, the edges of each slide were sealed with lacquer (e.g., nail polish) or other adhesive to prevent the ultra-thin nitrocellulose substrate from separating from the glass upon exposure to aqueous solutions. When colloidal nitrocellulose is applied to nylon slides, acetate film or other plastic surfaces, it requires no adhesive and binds avidly. Slides may be composed of almost any translucent material as long as the amyl acetate does not react with the surface to discolor it or alter its properties. Certain types of plastics become opaque when exposed to amyl acetate and are not suitable for use with that solvent system. In alternative embodiments, the colloidal nitrocellulose may be suspended in other volatile organic solvents besides amyl acetate before application to a nylon, glass or other translucent slide.

An advantage of the translucent nitrocellulose surface is that the progress of the probe binding reaction may be examined by looking through the translucent lower surface of the slide. This allows more effective probe binding to occur. The progress of the probe-target binding reaction may also be monitored in real time through the underside of the slide.

GRABBER™ Slides

Certain embodiments may concern devices and methods of use of translucent coated slides, e.g., GRABBER™ slides, prepared as disclosed above. GRABBER™ slides provide for an easy to use method for detecting and identifying one or more targets of interest in a solution assayed on the surface of a slide. Slides may be provided pre-spotted with capture probes (e.g., antibodies) affixed ready for immediate testing. Alternatively, users who want to prepare their own 2D-spotted array may in 1 hour prepare a manual array for testing.

In certain preferred embodiments, the method of detection utilizes a primary antibody capturing an antigen target to form an immunocomplex with a second antibody that is biotinylated or otherwise labeled. The first antibody probe is affixed to a translucent nitrocellulose-coated slide (GRABBER™) and during the entire procedure remains affixed to the surface of the slide.

The immunocomplex, if formed, may be detected using a well-known enzyme activated bioluminescent process. For example, horseradish peroxidase (HRP) catalyzes the breakdown of peroxide and produces an intense light if luminol is added to the solution during the breakdown process. Luminol forms an unstable free radical intermediate that results in the release of photons at 430 nm. By conjugating HRP to streptavidin (SA), the immunocomplex may be detected with an appropriate and sensitive photon detector. Biotin and streptavidin rapidly combine to form this light producing complex.

Typical protein (e.g., antibody) probes may be spotted with 1 to 5 nl fluid per spot at a concentration of 0.05 to 0.2 mg/ml. Where primary antibodies are attached to a slide, matrix array or other surface, secondary tagged antibody, such as biotinylated antibody, may be provided in a dilution buffer premixed at an optimized concentration for target detection with pre-spotted slides. The optimized concentration may be determined empirically by the user. Secondary antibodies may be diluted in dilution buffer to a concentration typically in the range of 5 to 30 μg/ml.

A TOAD™ (total optical assay device) may be provided with software to allow the user the means to read, interpret and record signals at the surface of a slide. An image file and report may be saved or printed for subsequent analysis and comparisons.

GRABBER™ slides may be coated with a translucent nitrocellulose substrate (see U.S. patent application Ser. No. 10/373,546, filed Feb. 24, 2003) that avidly and immediately binds proteins, carbohydrates and/or oligonucleotides, strongly affixing them to the coated surface while preserving the functionality and 3D structure of the bound molecule. No other chemical process is required to affix the probes to the slide surface, making the 2D array procedure simple and fast. A hydrophobic surface with a superior contact angle is well suited for compact robotically printed arrays.

The bioluminescent reaction is long lasting and the signal may be acquired over prolonged periods, allowing for extraordinary sensitivity in detection. Many targets may be simultaneously identified in a single sample. The proper primary probe antibody and biotinylated secondary antibody should strongly interact with the target for adequate detection. Preprinted slides and reagents provided are optimized to produce high quality detection. The skilled artisan will realize that translucent coated slides are merely one preferred alternative for detection of target binding and that other alternatives, such as the magnetic beads discussed below, may be used with an optical detection system.

For sample measurement using slide substrates, a stage designed to hold in place a spotted slide may be produced and affixed to the instrument housing over the CCD camera so that light producing spots in an array may be analyzed.

Reconfigurable Microarrays

In certain embodiments, reconfigurable microarrays may be produced by using small linker molecules, such as aptamers or affibodies, bound to the surface of a solid matrix. Aptamers are oligonucleotides derived by an in vitro evolutionary process called SELEX (e.g., Brody and Gold, Molecular Biotechnology 74:5-13, 2000). Aptamers may be produced by known methods (e.g., U.S. Pat. Nos. 5,270,163; 5,567,588; 5,670,637; 5,696,249; 5,843,653) or obtained from commercial sources (e.g., Somalogic, Boulder, Colo.). Aptamers are relatively small molecules on the order of 7 to 50 kDa. Because they are small, stable and not as easily damaged as proteins, they may be bound in higher numbers to the surface of a solid matrix. This effectively amplifies the number of probe reactive sites on the surface of an array.

Affibody® ligands (U.S. Pat. No. 5,831,012) are highly specific affinity proteins that may be designed and used like aptamers. Affibodies may be produced or purchased from commercial sources (Affibody AB, Bromma, Sweden). Aptamers and affibodies may be used in combination with antibodies to increase the functional avidity of translucent or non-translucent solid matrices for probe molecule binding. Increased binding in turn results in an increased signal strength, greater signal-to-noise ratio, more reproducible target molecule detection and greater sensitivity of detection.

Reconfigurable microarrays may be used in combination with two antibodies and a capture probe. The capture probe may be an affibody, aptamer or any other probe capable of binding one of the antibodies. Both antibodies should selectively bind to a target cell, molecule or antigen.

The effectiveness of binding is increased if the capture probe binds to a portion of an antibody characteristic of the IgG class. Such probes would only require a small part of the antibody structure to be present in order to react and bind to an antibody-target complex. Larger targets, such as microbes or cells are covered with numerous antigens that may form very large complexes with antibodies. However, truncated IgG antibody fragments could interact with such large targets and still bind to an aptamer or affibody probe on the slide surface.

In certain embodiments, two antibodies are allowed to bind to the target in solution. Once target-antibody complexes are formed, the complex may be exposed to aptamer or affibody probe molecules on a reconfigurable matrix array. The probes may bind to a first antibody, while the second antibody may be conjugated to a bioluminescent tag or other marker. The tagged complex may then be detected on the surface of the matrix array, using optical detection or any other known detection method.

For example, an aptamer may be tailored to specifically bind to the Fc portion of mouse IgG with high affinity. Samples containing target molecules of interest may be allowed to interact in solution with a mouse antibody specific for an antigen of interest. The sample is mixed with a different biotinylated or otherwise tagged second (non-mouse) antibody that binds to a different epitope on the same antigen. The target antigen bound to the first and second antibodies is exposed to the aptamer microarray. The anti-mouse aptamer affixes the complex to the solid matrix. After extensive washing to remove unbound tagged antibodies, the complex containing tagged antibody that is attached to the matrix array surface is detected.

The skilled artisan will realize that many variations on this scheme may be used. For example, in alternative embodiments, a first antibody may be used in conjunction with multiple tagged second antibodies, each of which binds to a different epitope of the target molecule. This may occur, for example, where the available second antibodies are polyclonal antibodies. Alternatively, use of more than one second antibody with affinity for the same antigen may improve the sensitivity of detection. In another alternative, one second antibody may bind to a class of targets (for example, all E. coli bacteria) while a second antibody binds to a specific subclass (e.g., E. coli strain O157:H7).

In a non-limiting example, to detect Listeria monocytogenes, an IgG mouse anti-Listeria m. antibody may be incubated with a food sample of interest at an appropriate concentration (typically 1 to 50 μg/ml). A rabbit (or other non-mouse) biotinylated secondary anti-Listeria m. antibody (1 to 50 μg/ml) may be added and incubated for 5 to 30 minutes. The sample with both antibodies may then be applied to the array containing anti-mouse IgG aptamers. After a short interval (approximately 15 minutes) the array may be washed so that only mouse IgG and rabbit biotinylated antibody complexed with Listeria m. is retained on the array. A solution of HRP-conjugated strepavidin or other indicator applied to the surface may then reveal the presence or absence of an anti-Listeria m. antibody complex affixed to the surface.

### Magnetic Beads

In certain embodiments, the probes, antigens or other ligands of interest may be attached to magnetic beads for separation and/or detection of target binding. Additional details of protocols for use with magnetic beads are disclosed in the Examples section below. In preferred embodiments, the probes, antigens or other ligands attached to the beads may be proteins or peptides, such as antibodies, antibody fragments, antibody binding proteins (e.g., protein A) or target antigens. Processes for the coupling of molecules to magnetic beads or a magnetite substrate are well known in the art, i.e. U.S. Pat. Nos. 4,695,393, 3,970,518, 4,230,685, and 4,677,055. Attachment may be either covalent or non-covalent. A number of potential chemical cross-linking agents are well known in the art, including EDC, dinitrobenzene, bisimidates, N-hydroxysuccinimide ester of suberic acid, dimethyl-3,3′-dithio-bispropionimidate, 4-(bromoaminoethyl)-2-nitrophenylazide, disuccinimidyl tartarate and azidoglyoxal.

It is envisioned that the magnetic particles employed may come in a variety of sizes. While large magnetic particles (mean diameter in solution greater than 10 μm) can respond to weak magnetic fields and magnetic field gradients, they tend to settle rapidly, limiting their usefulness for reactions requiring homogeneous conditions. Large particles also have a more limited surface area per weight than smaller particles, so that less material can be coupled to them. In preferred embodiments, the magnetic beads are less than 10 μm in diameter.

Ferromagnetic materials in general become permanently magnetized in response to magnetic fields. Materials termed “superparamagnetic” experience a force in a magnetic field gradient, but do not become permanently magnetized. Crystals of magnetic iron oxides may be either ferromagnetic or superparamagnetic, depending on the size of the crystals. Superparamagnetic oxides of iron generally result when the crystal is less than about 300 angstroms (Å) in diameter; larger crystals generally have a ferromagnetic character.

Dispersible magnetic iron oxide particles reportedly having 300 Å diameters and surface amine groups are prepared by base precipitation of ferrous chloride and ferric chloride (Fe²⁺/Fe³⁺=1) in the presence of polyethylene imine, according to U.S. Pat. No. 4,267,234. These particles are exposed to a magnetic field three times during preparation and are described as redispersible. The magnetic particles are mixed with a glutaraldehyde suspension polymerization system to form magnetic polyglutaraldehyde microspheres with reported diameters of 0.1 μm. Polyglutaraldehyde microspheres have conjugated aldehyde groups on the surface which can form bonds to amino containing molecules such as proteins.

While a variety of particle sizes are envisioned to be applicable in the disclosed method, in a preferred embodiment, particles are between about 0.1 and about 1.5 μm diameter. Particles with mean diameters in this range can be produced with a surface area as high as about 100 to 150 m²/gm, which provides a high capacity for bioaffinity adsorbent coupling. Magnetic particles of this size range overcome the rapid settling problems of larger particles, but obviate the need for large magnets to generate the magnetic fields and magnetic field gradients required to separate smaller particles. Magnets used to effect separations of the magnetic particles need only generate magnetic fields between about 100 and about 1000 Oersteds. Such fields can be obtained with permanent magnets that are preferably smaller than the container which holds the dispersion of magnetic particles and thus may be suitable for benchtop use. Although ferromagnetic particles may be useful in certain applications of the invention, particles with superparamagnetic behavior are usually preferred since superparamagnetic particles do not exhibit the magnetic aggregation associated with ferromagnetic particles and permit redispersion and reuse. In a preferred embodiments, a pair of spherical magnets may be juxtaposed to a container, such as a 1.5 ml Eppendorf tube, and used to collect magnetic beads on the side of the tube. The use of spherical magnets provides a more intense localized magnetic field, which facilitates separation of magnetic beads from solution.

The method for preparing the magnetic particles may comprise precipitating metal salts in base to form fine magnetic metal oxide crystals, redispersing and washing the crystals in water and in an electrolyte. Magnetic separations may be used to collect the crystals between washes if the crystals are superparamagnetic. The crystals may then be coated with a material capable of adsorptively or covalently bonding to the metal oxide and bearing functional groups for coupling with proteins or other ligands. Commercial sources of magnetic beads are also known in the art and may be used, for example Dynal Biotech (Brown Deer, Wis., USA).

In preferred embodiments, a magnetic cage may be used to facilitate the handling of ferrite particles during washing and reagent change steps. The cage may be produce by stacking neodymium ferrites in a column on opposite sides of a 50 ml conical flask. The cage consists of two blocks of non magnetic material (plastic) with a central hole just large enough to accommodate the diameter of a 50 ml straight wall conical tube.

The upper block of the cage is centered over the lower block and aligned so as to form a cylinder with the neodymium magnets forming a column nearly in contact with the wall of the conical tube when it is inserted in the cylinder. The ½ inch magnets with their strong field pull ferrite particles to the inner wall of the tube in the sorting process allowing the operator to then discard spent reagents or wash solutions, and re-suspend the ferrites in new reagent by removing the tube from the magnetic cylinder.

### Imaging Cassette Honeycomb Aluminum Core

In an exemplary embodiment, a disposable imaging honeycomb center cell cassette may be used for imaging and measuring luminosity on optical imagers, such as the TOAD™ discussed above. It may be positioned and held in place by locking pins on a stage to provide proper orientation of the cassette to the CCD camera. The lower surface (bottom) of the cassette is an optically clear window affixed to the honeycomb aluminum core. The opaque upper (top) surface of the cassette is marked to form a template for filling individual cells within the honeycomb. Its interior surface affixed to the top of the honeycomb is highly reflective, sending photons back to the sensor that would have otherwise escaped detection.

Constructing the cassette involves cutting of the skins to proper size and pattern cutting of aluminum honeycomb into squares measuring approximately 2.75×2.75 inches square. The bottom clear layer is die cut with locking holes punched before it is affixed to the aluminum honeycomb. The upper skin template that provides guidance to the operator for filling cells is affixed in a last step.

Affixing the lower skin to the honeycomb may be done by applying adhesive (e.g. activated fiberglass resin) first to the lower surface of the honeycomb, and then with caution to avoid smearing the optical window, placing the honeycomb on the lower skin to bond. Secure bonding of the aluminum to acetate was easily accomplished with fiberglass resin and catalyst by lightly coating the lower edge of the honeycomb dipped in a shallow tray of resin and then letting it rest undisturbed without any weighting on the acetate.

Aluminum honeycomb may be cut from stock 0.250″×48″×96″ ¼ inch cell 5.2 density non-perforated core visual grade for lighting product with attention to the pattern required, recognizing that horizontal cuts bisect only cells, and vertical cuts bisect cells and nodes in straight lines. The cut tolerance requirement is to stay within the ¼ cell row or vertical column initiated for the entire length of each cut.

### Upper and Lower Skin

In preferred embodiments, the lower skin is die-cut polyester clear 7 mil sheeting 2.64×2.64 inches square precisely the same size as standard Avery label 5196. The upper skin is tag stock with the lower foiled side affixed to the aluminum honeycomb also measuring 2.64×2.64 inches square die cut to the size of the standard Avery label 5196. The upper surface is either printed with the template pattern, or has an Avery 5196 printed label affixed. The template holes are KISS punched to the foil so that a pipette tip can easily be pushed through the upper skin and contents dispensed to the cell.

Adhesive Binder

There are several possible adhesives that could be used. Either a polyester or epoxy resin should work well. The binding requirements are minimal so the greater strength afforded by epoxy resins seems unnecessary. Polyester resin has been used successfully. The resin should be mixed with powder filler to thicken the resin during the cure period so that less of the resin will flow onto the polyester sheet as it sets up. Aluminum powder is recommended because it also renders the adhesive totally opaque, minimizing cross light contamination from one cell to the next (under the door leakage).

Cell Numbering and Configuration:

The opaque upper template affixed to the cassette provides to the operator a visual guide for loading samples into wells. Each cell in the cassette has an address. The address is by row and number in that row, although alternative numbering schemes may be used.

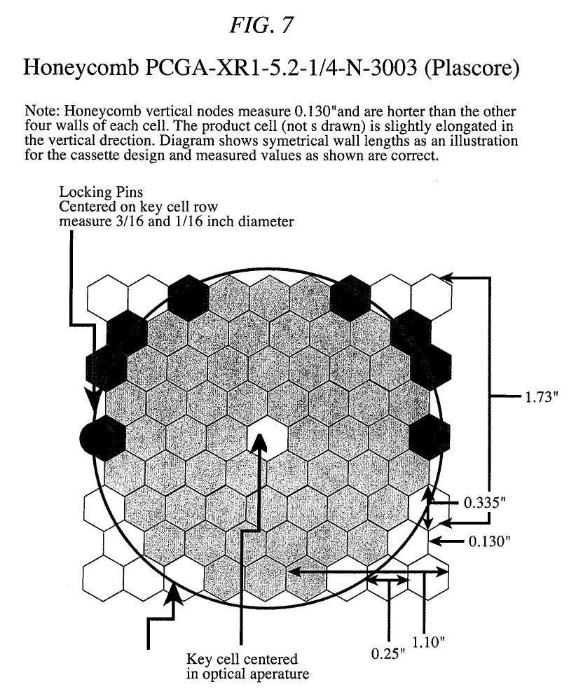

In the illustrated exemplary embodiment, there are 9 rows designated as A, B, C, D, E, F, G, H and I, and numbers for each cell's position in the row. For example the centered cell's address is E4. A loading template may be printed to the upper surface of the cassette directing the operator to load cell samples and is removed before imaging (see FIG. 7).

Loading Template Measurements:

In an exemplary embodiment, the upper template for loading is the same size as a standard Avery 5196 label and measures 2.64 inches square, printing 9 templates in an 8.5×11 inch sheet of pre cut tag stock. There are 55 cell access ports on the template. Access ports are numbered and identified by the row and number for a sample.

### Image Sensing and Data Analysis

In various embodiments, positive assay results may be detected by, for example, analysis of an optical signal emitted by a chemiluminescent target-ligand complex, as exemplified in Example 1. Various methods and apparatus for optical signal detection and analysis are known in the art and may be used in the claimed methods. For example, complementary metal oxide semiconductor (CMOS) image sensors are used in digital cameras and are increasingly found in a variety of analytic instruments. CMOS image sensors are improving in quality and are challenging and replacing charge-coupled device (CCD) imagers for detecting low level spectral images.

Most modem light detectors are designed to capture a spectral signal by presenting a two-dimensional array of sensitive photodiodes towards a target. The photodiodes are designed to produce current when exposed to light, and the resulting current may be analyzed in various ways. Modem sensors convert the analog photodiode signal to a digital signal format that may then be stored and processed for later analysis. High-resolution digital pictures may be produced pixel by pixel with an appropriate source of light, an optical system, an image sensor, and a computer. Using such a system, photographic pictures may be obtained in either monochromatic or color formats.

However, a photodiode will produce an analog output signal that correlates with the energy striking the photodiode array only in special circumstances, such as when the target is illuminated by monochromatic light at a particular wavelength. Even though the output signal of a photodiode is essentially linear with respect to the illumination applied to the photodiode, the signal value for a pixel does not generally correlate accurately with the photon flux. This is because the quantum efficiency (QE) for converting the photon flux to a photodiode electrical energy varies with certain factors. In addition, in most cases more than a single wavelength of light will strike a photodiode.

Every photodiode has a certain QE value that will vary with factors such as wavelength and temperature. Photon flux represents the electromagnetic energy striking the surface of a two-dimensional array, and the QE represents the capability of the photodiode to convert that energy into electrical energy. QE is usually expressed as a percentage of the energy flux, equaling some percentage less than 100 percent. Because QE varies greatly with the wavelength of light illuminating a photodiode, comparisons of a signal at one wavelength to that at another are difficult to interpret unless the QE factors are known for all wavelengths that apply. Further, most image sensors are designed by manufacturers to produce images that approximate the equivalent of what would be seen on a film or by the human eye. Manufacturers may provide access to the raw digital information for every pixel, but image sensors generally process that information before it is available for analysis to better render the “life-like” colors and intensities that represent human visual expectations. For these reasons, the data produced by an image sensor generally does not directly relate to the photon flux that impinges upon the photodiode array of the sensor. This factor limits the usefulness of image sensors for analytic purposes.

Image Sensor Operation

Investigators using a photodiode detector may incorrectly assume that the digital data acquired from the detector correlates with the photon flux striking the detector because increases in the intensity of the signal out will generally directly correlate with increases in signal input in a particular wavelength or band-width. Because photodiodes are very linear in output, increases in the photon flux at different wavelengths over the photodiode surface will, over the photodiode's dynamic range, produce a linear output signal. However, the output data will not correlate with the photon flux if the QE is variable over the range of wavelengths that are striking the photodiode. For a particular number of photons striking a photodiode over a time period, a larger current will be produced by the photodiode at a first wavelength than a second wavelength if the QE for the photodiode is higher for the first wavelength than the second wavelength.

Restricting a light source for analysis to a narrow band by filtering or by using a laser light source generally will not resolve accuracy issues. Emission spectra that are evaluated using image sensors may be very broad even if the excitation source has a narrow wavelength range. For this reason, the shape of the QE curve for a photodiode should be carefully considered in evaluating output data from an image sensor.

With regard to the choice of image sensors, CMOS imager sensors are fundamentally different from charge-coupled devices and are increasingly used in microscopy and diagnostic instruments because they are cheaper to build and require considerably less power to operate. CCD cameras are no longer clearly superior in low intensity light situations, which had been true in the past. CMOS images now rival traditional color imaging methods on film and are easily manipulated.

The images may be transferred from one processor to another as a digital file in a variety of formats preserving the arrayed data pixel address. Manufacturers have devoted considerable energy reproducing “life-like” color image sensors using various color filters and interpolation methods to enhance a digital image rendering colors very close to the human eye experience. However, the pleasing “life-like” color pictures obtained with a color CMOS image sensor are not as useful for analytic procedures. Similar issues exist with monochromatic images unless the image is produced by light at single wavelength, which is rarely true.

In a non-limiting example of a color CMOS image sensor that may be utilized, an image sensor includes an imaging array. The imaging array is comprised of a large number of pixels arranged in a two-dimensional array. There is a filter associated with each of the pixels in the imaging array. The image sensor will also generally contain electronics relating to the processing and transmission of signals generated by the imaging array, including analog signal processing, analog to digital conversion, and digital logic.

The photodiode array in a CMOS color image sensor is blanketed by an ordered thin layer of polymeric filters, such as in a conventional Bayer RGB (red-green-blue) two-dimensional array. Each filter is sized to fit over an individual photodiode in a sequential (Bayer) pattern to capture color information from a broad bandwidth of incident illumination. In an RGB array, a heavy emphasis is placed on the green filters to address the human visual maximal response at 550 nm. There are 2 green filters for every red and for every blue filter. However, even though the human eye is more attuned to the green 550 nm region, yellow is generally a better choice with regards to QE factor.

CMOS image sensors and the integrated circuits that define the active pixel array are inherently monochromatic (black and white) devices that respond only to the total number of electrons striking the photodiodes, not to the color of light. Color is detected either by passing the light through a sequential series of filters (such as red, green, and blue filters), or with miniature transparent polymeric thin-film filters that are deposited over the pixel array.

Active pixel sensor (APS) technology is the most popular design for CMOS image detectors. In addition to a photodiode, each pixel (or imaging element) includes a triad of transistors on its surface that convert accumulated electron charge to a measurable voltage, reset the photodiode, and transfer voltage to a vertical column bus. The photodiode thus occupies only a fraction of the pixel area. The photodiode area encompasses an area equal to 30 to 80 percent of the total pixel area for most CMOS sensors. This area occupied by the photodiode is the area that absorbs photons, while the other parts of the pixel are relatively opaque, blocking, reflecting, or absorbing light. The photodiode area or window is referred to as the “aperture” or “fill factor” of the pixel or image sensor. A small aperture or fill factor results in a significant loss of sensitivity and a corresponding reduction in the signal to noise ratio and leads to a reduction in the dynamic range of the sensor.

CMOS image sensors can be utilized to produce pictures based upon the signals produced when photons strike the photodiode surface associated with each pixel in an active pixel sensor array. The pixel signals are processed to form the total picture either in monochrome (black and white) or color. Monochrome CMOS imager sensors do not have color filters over the photodiode portion of the pixel. However, color CMOS imagers, even with standard Bayer pattern filters, generally are more sensitive than monochromatic CMOS imagers. While it may appear that inherently monochromatic CMOS photodiode without filters would be more sensitive because some light passing through a filter is absorbed and never reaches the photodiode in a color filtered photodiode, this is not generally true. This assumption does not fully take into account the effect a filter has on the QE for a photodiode, which might enhance certain signals, and ignores the advantages provided by microlenses in color photodiode architecture, which are described below. Monochromatic CMOS image sensors do not have a color filter and they do not normally have microlenses over each pixel. These are important factors that make monochromatic imagers less attractive than color CMOS image sensors with regard to imager sensitivity.

A small section of an imaging array of a color image sensor that may be utilized is comprised of four pixels, each having a filter. The filters have colors based on the choices made for the array. For each pixel, there is a portion that comprises the photodiode, the area of the photodiode being only a fraction of the total area. The image sensor will detect only the portion of the light falling on the photodiode area.

In color imagers used in analysis, one possible approach would be to construct imagers by carefully selecting filters and photodiodes to produce QE factors for a given bandwidth that is approximately constant. By combining an appropriate number of photodiodes in an array with chosen filters, the measurement of light energy would be more accurate. The filters chosen could assist in leveling and improving upon the QE factors. However, in practice the filters and photodiodes are chosen for other purposes, with a goal of producing the most visually pleasing images. In order to improve upon the quantum efficiency and spectral response, several CMOS manufacturers use color filter arrays based on the primary subtractive colors, cyan, magenta, and yellow (CMY), instead of the standard additive primaries red, green, and blue (RGB). CMOS image manufacturers generally use either Bayer RGB or Bayer CMY patterns that have been selected for photographic imaging.

For RGB and CMY filters, an imaging array is divided into small arrays of filters, with each such array of filters having the same filter pattern. An RBG filter array contains two-by-two arrays of filters, with each array containing a red filter and a blue filter for two diagonal pixels and two green filters for the remaining two diagonal pixels. A CMY filter array also contains two-by-two arrays of filters, with each array containing a cyan filter and a magenta filter for two diagonal pixels and two yellow filters for the remaining two diagonal pixels. Many other filter colors and patterns are possible, and any filter pattern may be used.

In contrast to monochrome image sensors, color CMOS image sensors also contain microlenses that effectively direct photons to the photodiode aperture. The bubble lens, generally including an anti-reflective coating, can effectively increase the surface area of a photodiode by a significant amount, approximately 60 percent in certain applications. The microlenses substantially increase the effective fill factor and may more than compensate for filters that cut down on the total light that can reach the photodiode.

In certain embodiments, there may be microlenses in an image sensor. Within the image sensor, there are multiple pixels. Each pixel contains an active portion, with the active portion including only a portion of the pixel area. In order to compensate in part for the light energy that would not normally strike the active portions, each of the pixels has an associated microlens. The function of each microlens is to focus more light energy on the active portion and thus to allow measurement of a larger percentage of the incident photon flux.

Image Sensor Optimization

Three primary mechanisms that reduce or hamper photon collection by the photosensitive area of an image sensor are absorption, reflection, and transmission. These factors are wavelength-dependent in nature, and define in part the quantum efficiency (QE) of the image sensor. For example, reflection and transmission of incident light occurs as a function of wavelength, with a high percentage of shorter wavelengths below 400 nm being reflected. Shorter wavelengths are absorbed in the first few microns of the photosensitive region but the longest wavelengths exceeding 650 nm often pass through the photosensitive region.

By examining the QE wavelength dependence curves for each filter type used in an image sensor, the output signal proportional to the photon flux can be determined for any wavelength or interval of interest, including those pixels for a monochromatic image sensor. In many cases every pixel in an array is essentially identical to its neighbor except for the kind of filter (CMY, RGB, other pattern, or no filter). The effect of a filter is either to increase or to reduce the photodiode energy output for a given photon flux. The effect on the signal is wavelength dependent. The QE is the variable in the output signal that should be factored out of the equation if fair comparisons are to be made across the imaging array for each and every pixel.

In a CMOS imager, the pixel signal is obtained for each pixel as raw data after the analog to digital converter transforms the value for a set time interval. If QE is expressed as a fraction, the pixel signal is directly proportional to the product of the QE and Photon Flux:

Pixel Signal=Constant×QE×Photon Flux

If raw data can be normalized according to the appropriate QE, digital values can be created that may be used for more accurate subsequent analysis. The pixel value for a color CMOS image sensor may be obtained before the on chip conversion occurs and the value normalized by multiplying each signal value for a particular color filter by the inverse QE. For a relatively narrow bandwidth, the QE may be treated as a constant depending upon the wavelength and filter type used. In one example, the Bayer CMY pattern over the range 550 to 650 nm for a Kodak 1310 color CMOS image sensor provides a QE of approximately 46 percent, and then drops linearly from 650 nm to 5 percent at 990 nm, approximately 0.6 percent every 5 nm. In addition, the Magenta and Yellow filters are very similar over the range from 630 nm to 990 nm.

For a particular example with a CMY pattern Kodak 1310 color image sensor at 670 nm, the QE values are as shown in Table 3. A pixel with a yellow filter would have its digital raw data multiplied by 2.38, a magenta filter pixel by 2.27, and a cyan filter pixel by 7.69. In this embodiment, the signal for every pixel is effectively transformed to a numeric value that is directly proportional to the actual photon flux. It is noted that Table 3 only contains the QE for the image sensor when light of a particular wavelength (670 nm) strikes the image sensor. The QE for any other wavelength of light will vary.

TABLE 3
Quantum Efficiency and Normalization Factors
for Kodak 1310 Image Sensor at 670 nm

| | Quantum Efficiency | |
|---|---|---|
| Filter Type | (%) | Normalization Factor |
| Yellow | 43 | 2.38 |
| Magenta | 44 | 2.27 |
| Cyan | 13 | 7.69 |
| Monochrome (no filter) | 28 | 3.57 |
| Red | 35 | 2.86 |
| Green | 5 | 20.0 |
| Blue | 3 | 33.3 |

QE Factor Correction

Corrections to account for differences in QE may be made based upon known QE factors for a particular filter type and wavelength. However, an image sensor may also be utilized to automatically correct for differences in pixel QE. Each area of a sensor array, such as each filter quadrant, may be normalized to render every pixel in the quadrant optimally tuned for photon flux in real time. No corrections are made if the pixels and filters are all of the same type, as, for example, the YYYY, MMMM, and CCCC filter patterns. A correction may be made if there are two or more filter types in the array (e.g., filter patterns such as YYYC, RGB Bayer, or modified CMY Bayer). A method of auto correcting for QE may be used for any combination of two or more filter and photodiode types and such method corrects to normalize the 4 pixels in a quadrant so that each pixel produces an equivalent output signal.

If the pixels are tightly packed in a quadrant relative to the change in photon flux over a given region of the array, then it can be assumed that the same number of photons are striking each pixel in the quadrant at any given moment. With the currently available high-resolution sensors, and with anticipated future improvements in resolution, the assumption that neighboring pixels in any given quadrant experience identical photon flux is appropriate. Using this assumption, each of the pixels in the quadrant should produce the same output. Accordingly, auto correction may be used to make adjacent neighbors in each filter quadrant identical. The most sensitive pixel type in a quadrant may be used to factor out QE and wavelength differences, which simplifies the problem of correcting for wavelength and bandwidth dependence. In one example, once a background correction factor is determined for the most sensitive pixel in a quadrant, the same background correction factor may be assumed for each of the other pixels in the same quadrant. Auto correction also reduces or eliminates problems related to temperature variations for different filter and photodiode types. An automated method for threshold and normalized luminosity assignments is discussed in the following section.

In another example, an array of an image sensor comprises multiple filter quadrants. Two or more filters are used in each quadrant of 4 pixels. In each quadrant of 4 pixels, the average analog to digital converted signal output for each filter and photodiode type is determined. If, for example, there are 3 yellow filtered pixels and 1 cyan filtered pixel in the quadrant, the average for the 3 yellow pixels is determined first. The output value for the yellow pixels is then compared to the value for the cyan pixel to determine which output is numerically greater. Under the embodiment, there is an assumption that all 4 pixels receive equivalent photon flux. The highest output value is assigned to all four pixels in the quadrant. The next quadrant in the array may then be corrected in the same manner, with the process continuing until the entire array has been assigned corrected output values to correlate with photon flux.

The process of auto correction is repeated over time as an image sensor is used to record images. In yet another example, the wavelength of light received by an image sensor may change from a first wavelength to a second wavelength. A first type of filter may provide the highest QE for the first wavelength, while a second type of filter provides the highest QE for the second wavelength. The change in wavelength may be included in the calculation process and therefore auto correction for changing light can be made in real time.

It is not necessary to know in advance the QE for each filter type to auto correct for QE differences. Auto correcting the sensor based on the photon flux at the time an image is obtained optimizes the photo image to correlate with photon flux. This method of correcting the signal removes temperature and wavelength dependence differences for different filter types and can be implemented using software. Such method thus automatically corrects for a broad band signal impinging upon an image sensor. In yet another example, the digital signals produced by an image sensor auto corrected for photon flux may be rendered to a gray scale image for subsequent visualization in a monochromatic representation.

Image Sensor Calibration

The optimization of an image sensor may include a calibration step. The calibration may be accomplished by illuminating the color filters and photodiodes with light of known wavelength and intensity. For a color CMOS imager, the raw data for each filter is obtained and compared to expected values. From the resulting comparison, the QE and the multiplier (normalization factor) that is required to obtain the equivalent output signal for each color filter used for each and every pixel in the array may be obtained.

Optimized signals obtained using QE factor conversions can more accurately relate the signal to the photon flux, and therefore more precisely characterize events, such as the optical events related to excitation-emission spectra or absorption phenomena in a chemical reaction. Both sensitivity and accuracy are enhanced by properly converting the signal to account for QE factors. Using a standard CMOS imager (such as a Kodak 1310 color CMOS image sensor,) raw data produced may be processed for signal optimization. The signal is converted to a numeric value that correlates with the photon flux incident upon the imager. This process can be applied to either a color or monochromatic CMOS imager sensor to render the signal proportional to photon flux.

Data processed may be rendered for visualization, such as via a gray scale standard (0 to 255 monochromatic) to producing a black and white image that correlates with the actual photon flux. The visual image of the data is superficially equivalent to a gray scale monochromatic image sensor, but for an equivalent luminance will be more intense than a image produced by a monochromatic non-transformed CMOS counterpart because color CMOS chips are generally more sensitive than monochrome chips. A color image sensor generally provides a better signal and is more sensitive than a monochromatic imager because the pixel photodiode filters improve upon the QE for the photodiode. The filtering of light by a color image sensor may be corrected using the QE factors to convert the signal to a number that is directly proportional to the photon flux. Further, advantage then is taken of the color filter's microlens effectively amplifying the aperture for the photodiode.

Illustrations of Processes

In a non-limiting example of a process for calibration for optimization of an image sensor, a light of a known wavelength and intensity is produced. With a known intensity, the photon flux on each pixel is known, which would be the output if the QE of a pixel were 100 percent. The known light is directed on an image sensor. The output of each pixel of the image sensor is obtained. The output of the image sensor then can be compared with the actual photon flux. Using the comparison, the quantum efficiency of the pixel can be calculated, and then a normalization factor is calculated based upon the quantum efficiency. For a color CMOS image sensor, the comparison and calculation can be done for each filter color, resulting in a normalization factor for each filter color. In other embodiments, the comparison and calculation can be made for each pixel of an image sensor or for sectors of pixels of an image sensor, resulting in normalization factors that apply for certain portions of the image sensor. As the normalization factor varies for each wavelength of light that strikes the image sensor, the wavelength of the known light is varied and the process repeats for each needed wavelength.

In certain embodiments, the image sensor may be a color sensor containing an array of pixels, with each pixel having a filter. The filters may be arranged in quadrants, with each quadrant having a particular filter pattern. The outputs of each of the pixels within a first quadrant of the array may be obtained. The average output of for each filter type in the quadrant is then determined. In one example, if a filter quadrant is CMY pattern containing a cyan filter, a magenta filter, and two yellow filters, the cyan output, the magenta output, and the average of the two yellow outputs are determined. The outputs are then compared and the highest output is determined. The highest output is then assigned to each pixel in the quadrant. For example, if the average yellow output is the highest output for the CMY quadrant, indicating that, under the particular conditions, the yellow filter has the highest QE factor, then the average yellow output is assigned to each of the pixels in the quadrant. If there are more quadrants in the array, the output of the next quadrant is obtained and the process continues. Once the final quadrant has been corrected the process is completed and the corrected output for the array is available. The process can then be repeated over time to allow real time QE factor correction for the image sensor.

In an exemplary process for optimization of an image sensor, an image of an event is captured with an image sensor. Under one embodiment of the invention, the image sensor is a color CMOS image sensor utilizing a filter pattern such as a Bayer RGB or CMY pattern. The raw data for the image of the event is obtained from the image sensor. The raw data is non-optimized data that, due to the nature of the image sensor, will generally vary greatly from the actual photon flux that struck the image sensor. As the normalization factor depends on the wavelength of light, the wavelength is determined. The appropriate normalization factor is determined for each pixel based upon the wavelength of light. For one embodiment utilizing a color CMOS image sensor, a normalization factor for each lens color is used in normalization. Under other embodiments, the normalization factors may vary based on other factors. The raw data is then converted using the appropriate normalization factors for the pixels of the image sensor, thus producing an optimized data set that approximates the actual photon flux for the captured image of the event. Under an embodiment of the invention, an image may be produced using the converted data.

Fluorescence Detection

Fluorophores are frequently used to detect the presence or absence of a coupled reaction on a glass surface. Fluorescence detectors measure the intensity of the evanescent wave produced when a fluorophore is excited with a laser or other light source. Typically the laser is used to excite the fluorophore at its absorption peak and the detector is tuned to read the emission signal at a longer emission wavelength, which is characteristic of that particular fluorophore. The shift in wavelength between absorption and emission is referred to as the Stokes shift. Most fluorescence detection methods use fluorophores with a large Stokes shift so that the emission and absorption curves are well separated. With fluorophores that have a small Stokes shift, it is necessary to excite at a shorter wavelength than the optimal peak absorption maximum because of overlap between the emission and absorption curves. The signal emission intensity is reduced and the sensitivity for detecting target molecules is decreased. The need for a large Stokes shift also limits the choices of fluorophores that can be used.

Because the curves for absorption and emission are frequently very near to one another, accurate reading of the emission signal may be complicated. If the distance between the emission and absorption curves is small, it is difficult to separate the light from an emission spectrum from that of the absorption signal. Lasers with a narrow band at the absorption peak are frequently used with filters to cut out all light up to a certain critical point just below the emission spectral curve. By selecting an appropriate long pass filter, band pass filter, or combination of long pass and band pass filters, the emission signal can be observed in a narrow window, eliminating much of the interference from the excitatory light source. Interference from the excitatory light source is also avoided by aligning the detector and apparatus so that the emission signal can be read at a large incident angle to the excitation beam. Although filters eliminate most of the signal from the excitatory light source, they also cut out a significant portion of the evanescent (emitted) signal. Most band pass filters cut out as much as 40 to 50% of the emission signal. Long pass filters may cut an additional 10% of the emission signal.

Fluorescent detection is used in a number of common test methods. DNA hybridization is commonly analyzed in this manner, using an appropriate fluorophore coupled to a set of known oligonucleotides that hybridize to capture oligonucleotides affixed to a slide. Sandwich immunoassays also employ this method of analysis, either using a tagged secondary antibody that binds to a primary antibody, or using a secondary biotinylated antibody and an avidin-fluorophore as the tag. Many variations on this method are well known.

Various other types of light interference may occur in fluorescent detection. Light scatter occurs by reflection of the excitation beam, while light dispersion occurs by reflection and bending of the excitation beam. Scatter and dispersion may represent a large part of the light striking a detector. In general, when a substance (such as a protein, nucleic acid or other biomolecule) is affixed to the surface of a glass slide, it acts as a mirror to reflect and scatter light in a variety of directions. The amount of surface covered and the mass or density of the attached material may greatly affect the amount of scattered light. The chemical composition of proteins, oligonucleotides or polymers attached to the glass surface may also affect the scattered light. In addition, the material attached to the glass surface material may itself fluoresce. The glass used may also have surface irregularities that can affect the signals received by the detector. The energy absorbed across the glass may vary from one spot to another, making signal analysis very problematic. Such problems require the use of novel methods of fluorescent detection and/or data analysis.

Evanescent Emission and Scattered Light

Evanescent signals are generally very weak and light scatter is intense, making accurate quantitative detection of analytes problematic. Light scatter is frequently assumed to be eliminated by filters. However, scattered light is almost always present and can be a significant part of the total signal reaching a detector. Filters used to remove light scatter also remove much of the target emission signal, thereby decreasing detector sensitivity. Filters may also transmit a small amount of scattered light. If the scattered light is relatively large compared to the evanescent emitted light, the detected signal will be a combination from several sources, only one of which represents target molecule binding.

In an exemplary model of light scattering, two spots (e.g., different antibodies) are deposited on a glass surface. During a method to detect a target, one of the spots remains totally non-reactive. The other spot reacts with a target, such as a bacterial pathogen and/or other reagents. Target binding to the reactive antibody increases the mass attached to the spot and results in a larger surface area and a change in molecular structure at the spot. A mass effect has occurred. The light scatter from the reactive spot will be different from the light scatter before target molecule binding. A sensitive photon-counting detector could detect this difference in scatter. A variety of instruments, such as certain flow cytometers and turbidity meters take advantage of scatter to quantify the amount of material in a solution. Those instruments measure the angle of scatter for a beam of light impinging on a target material. The change in signal is the difference between the reference signal (S_{ref}) and signal 2 (S₂). The S₂ signal may have two components, a modified scatter signal plus a mass effect signal of the coupled pathogen. The signal from the reactive spot changes while the signal from the non-reactive spot signal is constant.

ΔS(non-reactive spot)=0

ΔS(reactive spot)=Modified(Sₚ)+M₁−S_{ref}

If the mass effect is sufficient to cause a large scatter effect, the fluorophore used for target detection could be eliminated. For example in DNA hybridization experiments, the mass attached to a surface using standard oligonucleotide probes (about 24 nucleotides in length) may be increased by a factor of 2 or more upon binding of target nucleic acids. Such a large change in mass may be detectable by monitoring light scatter instead of evanescent waves. In the case of a sandwich immunoassay with a biotinylated secondary antibody, another mass effect occurs when the biotinylated antibody binds to the pathogen. A third mass effect occurs when avidin-conjugated fluorophore binds to biotin.

The most sensitive signal may be obtained by subtracting the initial reference signal from the final captured signal, obtained after the fluorophore has been attached and excited. That signal represents the modified accumulated mass effects and the emission signal for the reactive spot.

ΔS(reactive spot)=Modified accumulated mass effects+Emission—S_{ref}

This method of analysis can be used with a CMOS imager or any known digital imaging method that allows storage of pixel images for subsequent processing. The signal obtained from each spot will contain more useful information and will show a more intense change upon target binding if a proper subtraction method is used. The scatter effect may be turned to an advantage in detecting target binding. Moreover, it is unnecessary to have fluorophore emission and absorption curves well separated, since spurious signals are subtracted out of the image. The full intensity of an emission signal may be measured without reducing emitted light by with filters.

A subtraction method also eliminates artifacts and defects that may derive, for example, from inhomogeneity (chips, flaws) in the glass slide surface. The non-reactive spots completely blank out and do not appear as a signal. Because CMOS imagers and pixel capturing devices in general exhibit a random, very low level noise there are limits as to what kinds of signals can be detected. At any given point in time, the baseline reference may exhibit a random number of spikes. A weak signal falling between two spikes would not normally be detected against this background noise.

The signal-to-noise problem may be improved if numerous images are captured and added one upon the other. Because the random spikes inherent in a detector such as a CMOS imager are constantly shifting about, accumulating the frame images will tend to average out the random noise. However a weak signal from the emission of an excited fluorophore does not change its pixel location. Therefore, an accumulated signal caused by target binding will increase with time. This method is similar to taking a photoimage of a distant star or galaxy, by tracking the object as it moves across the sky. The object of interest appears brighter against the background with time because the signal has accumulated at the same spot on the detector, while the background light averages out.

Method of Analysis

In an exemplary embodiment, a glass or nylon slide or other matrix array is secured on a stage. Before target molecule binding, an excitatory laser is focused on one end of the slide at an inclined angle about 30 to 40 degrees. The slide acts as a waveguide to conduct the excitatory light to spots, containing bound primary antibody, on the surface. A CMOS, CCD or other optical imager is used to capture the light signals. The imager may be located beneath the slide and aligned so that spots on the slide are directly above the imager and are sharply focused on the imager surface with optical lenses and apertures.

A number of pictures are taken. Each picture represents a single frame. For example 10 frames are taken using a 50 millisecond exposure. The exposure is selected so that the amount of light captured in a single frame is within the sensitive range for the camera. The 10 digital frames are then added to provide a reference set that is used for subtraction of unwanted (background) signals. The accumulated image is referred to as the calibration slide.

The same number of frames used to obtain the reference slide image are taken of the sample slide after binding of primary and/or secondary antibodies, enzyme and reagent, etc, using the same exposures. The cumulative set of frames is referred to as the sample slide image. The luminescent signal for each spot is determined by subtracting the reference slide image from the sample slide image. This process essentially eliminates background noise and matrix array artifacts, resulting in very sensitive detection of target molecules.

In alternative embodiments, pictures may be obtained in either still frame or video mode. A typical video frame runs at 2000 ms and captures 100 frames each for the reference and sample analysis. This method removes artifacts and non-reactive spots, leaving only those signals that represent target molecule binding to the array.

### Auto-Threshold Correction

A common problem in sample analysis has to do with making measurements and interpreting the meaning of values when no absolute standard exists. For example in measuring luminosity, because the measurement can not normally be obtained precisely when the reaction is initiated, and because the concentration of substrate is typically not known, only relative luminosity measurements are generally obtained and used in the analytic process. There are no absolute standards against which the signal can be measured.

Luminosity is frequently measured using a CCD camera with software. Each instrument will measure in terms of the absolute numeric value differently because of variations in the circuitry, optics, CCD chip and housing, etc. Even the same instrument will measure differently from moment to moment and scan to scan and on different days due to subtle changes in temperature and circuitry response, variations in solutions, scanning time, etc. making it difficult to interpret absolute values obtained in a given set of measurements.

For a particular instrument, when these differences in signal are relatively small compared to the numeric value obtained it may be possible to easily differentiate one sample in a set relative to others in the set, and to assign it to a population with a different distribution profile, for example as a negative or positive serum sample for a particular marker. However, in practice the differences in a measurement may be large from one experiment to the next relative to the absolute numeric value for the same or similar samples. This is especially true when the relative luminosity is very low in value and low level measurements are made.

By using a reference within a set of measurements, to which all other measurements are compared, it is possible to accurately state that a signal is either greater than, equal to, or less than that reference numeric value. And with the frequently observed broad dynamic range of over 1000 to a million in relative luminosity units, it is possible to measure relative luminosities to well within a few parts per 1000 units.

But with each new set of measurements, due to the change in absolute values, the relative luminosities of one set of measurements obtained by subtracting background measurement, or specific sample measurement, from that of another will not necessarily render the equivalent relative luminosity for a test sample. A method of differentiating one sample set in a series (the negative or normal set of sera) with a distribution curve that is different from that of another (positive or uniquely marked and differentiated sample set) is needed. And a method of making all instruments equivalent in their ability to differentiate signals in the same way is also needed. This can be done for any sample test, using only the measured luminosity for sample providing either the negative or positive reference set and background reference signal is also obtained in the scanning procedure.

Generally it is better to use as the reference the negative or normal set because a pool of samples may be prepared and used as the reference, closely approximating the average expected value for several samples simultaneously tested and averaged. Positive or uniquely marked sera may vary considerably depending upon the marker. For example, cattle infected with M. bovis with circulating antibody as the marker of interest may have very little antibody early in the infection. In a different animal or at a later time in the infection the antibody concentration may be very much larger or smaller in concentration. The positively infected animals should not be used for referencing. Negative (normal) sera are devoid of the antibody marker specific for M bovis and can be used as the reference.

Relative Luminosity Measurements

The Total Optical Assay Device (TOAD) described above, used with software, optics, and instrument housing loaded with a set of samples for analysis provides to the investigator a numeric value for each sample in a crib set. A crib set normally contains 12 or more wells, each containing either sample or a control solution that is scanned to capture an image in a set period of time (e.g., 10 minutes).

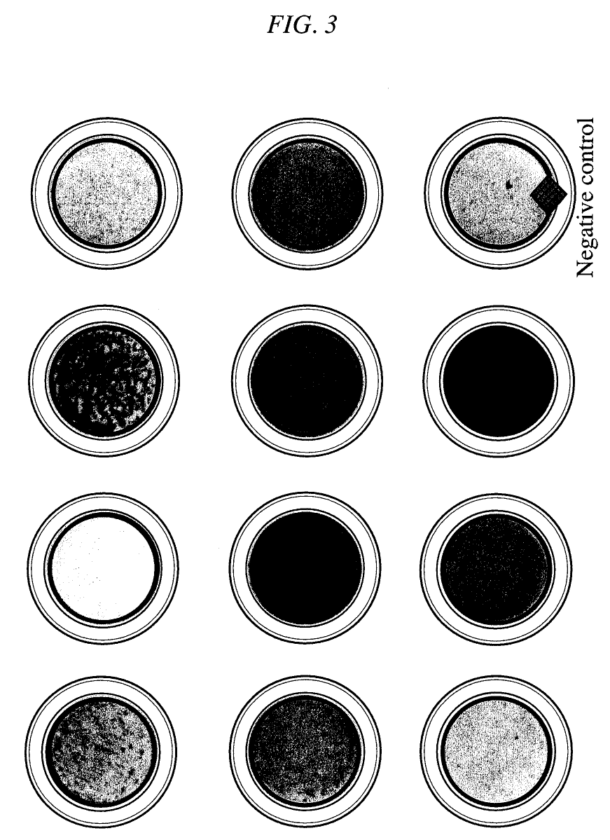

Normally several samples are scanned simultaneously. Background numeric values representing signal that would have been observed in the absence of sample (background) are obtained at the same time as samples are scanned by examining those areas in the CCD camera target area between the samples. The difference between the sample numeric value and background numeric value represents the relative luminosity for a particular test sample. A representative image for a set of 12 samples is shown in FIG. 3 with the pool of approximately 10 sera negative animals at the same substrate concentration used as a reference (negative control).

Because only 11 samples and the negative control can be processed in a single crib set assay, many sets are required to analyze a large number of samples. For each set, 11 additional samples are obtained and their numeric values are measurable relative to the negative control reference for that crib set. Table 5 shows the results for 9 consecutive assay sets with the relative luminosity for 99 different samples in a blinded study with uninfected and infected cattle sera.

The relative luminosity for each sample in a crib set provides useful information allowing the differentiation of a positive from a negatively infected animal using the referenced pool of negative controls. This is accomplished by assigning a threshold above which the sample is positive and below which it is negative. The threshold is obtained empirically for a given set of experimental conditions.

Threshold Assignments:

The threshold cutoff used to determine if a sample is either positively or negatively infected is determined empirically. Negative control reference values were obtained for each of the 9 crib sets and the background, NC, and NC relative luminosity values (by subtracting BG from NC).

The relative luminosity for pooled negative control sera is acquired. The values for NC_RL vary considerably. The ratio of the average of several sera measured individually plus a multiple standard deviation from the average relative to the pooled negative reference is a constant. The ratio is constant because the CCD image out put is directly proportional to photons striking its surface, and both the pooled sera reference and negative sample sera in a test are therefore affected to the same degree for a set of experimental conditions. A threshold is determined automatically by exploiting the relationship between the average expected relative luminosity value for a serum that is negative relative to the reference of pooled negative samples, and the observed value for a sample that may or may not be truly negative by adding to the average expected relative luminosity value a multiplier of standard deviations above the average that should include any negative sera sample. This can be expressed by the equation:

Threshold=Obs_{NC}+X(SD)−NC_{—}RL_{pooled reference}
Obs_{NC}=Observed negative control sample serum value
X=Multiplier
SD=Standard Deviation of several negative sample sera values
NC_RL=Negative Control pooled sera reference value

TABLE 5
ovine TB S&S Blinded Study Initial Screen

| | | | | Delta | | | False | | Actual | | Unblinded |
|---|---|---|---|---|---|---|---|---|---|---|---|
| Sample | Obs | BG | RL | NC | Pos | Neg | Pos | Neg | Neg | Pos | Assignments |
| 14-1 | 4034 | 3226 | 808 | −181 | 0 | 1 | 0 | 0 | 1 | 0 | Negative |
| 14-2 | 4488 | 3226 | 1262 | 273 | 0 | 1 | 0 | 0 | 1 | 0 | Negative |
| 14-3 | 4651 | 3226 | 1425 | 436 | 0 | 1 | 0 | 0 | 1 | 0 | Negative |
| 14-4 | 3460 | 3226 | 204 | −785 | 0 | 1 | 0 | 0 | 1 | 0 | Negative |
| 14-5 | 4170 | 3226 | 944 | −45 | 0 | 1 | 0 | 0 | 1 | 0 | Negative |
| 14-6 | 4260 | 3226 | 1034 | 45 | 0 | 1 | 0 | 0 | 1 | 0 | Negative |
| 14-7 | 4556 | 3226 | 1330 | 341 | 0 | 1 | 0 | 0 | 1 | 0 | Negative |
| 14-8 | 3923 | 3226 | 697 | −292 | 0 | 1 | 0 | 0 | 1 | 0 | Negative |
| 14-9 | 5205 | 3226 | 1979 | 990 | 1 | 0 | 0 | 0 | 0 | 1 | Positive |
| 14-10 | 4708 | 3226 | 1482 | 493 | 0 | 1 | 0 | 0 | 1 | 0 | Negative |
| 14-11 | 4372 | 3226 | 1146 | 157 | 0 | 1 | 0 | 0 | 1 | 0 | Negative |
| 14-12 | 2041 | 840 | 1201 | 594 | 1 | 0 | 0 | 0 | 0 | 1 | Positive |
| 14-13 | 1813 | 840 | 973 | 366 | 0 | 1 | 0 | 0 | 1 | 0 | Negative |
| 14-14 | 1116 | 840 | 276 | −331 | 0 | 1 | 0 | 0 | 1 | 0 | Negative |
| 14-15 | 1744 | 840 | 904 | 297 | 0 | 1 | 0 | 0 | 1 | 0 | Negative |
| 14-16 | 2208 | 840 | 1368 | 761 | 1 | 0 | 1 | 0 | 1 | 0 | Negative |
| 14-17 | 2784 | 840 | 1944 | 1337 | 1 | 0 | 1 | 0 | 1 | 0 | Negative |
| 14-18 | 3508 | 840 | 2668 | 2061 | 1 | 0 | 0 | 0 | 0 | 1 | Positive |
| 14-19 | 2280 | 840 | 1440 | 833 | 1 | 0 | 1 | 0 | 1 | 0 | Negative |
| 14-20 | 3173 | 840 | 2333 | 1726 | 1 | 0 | 0 | 0 | 0 | 1 | Positive |
| 14-21 | 2163 | 840 | 1323 | 716 | 1 | 0 | 1 | 0 | 1 | 0 | Negative |
| 14-22 | 1853 | 840 | 1013 | 406 | 1 | 0 | 1 | 0 | 1 | 0 | Negative |
| 14-23 | 4291 | 3713 | 578 | −418 | 0 | 1 | 0 | 0 | 1 | 0 | Negative |
| 14-24 | 4582 | 3713 | 869 | −127 | 0 | 1 | 0 | 0 | 1 | 0 | Negative |
| 14-25 | 5308 | 3713 | 1595 | 599 | 0 | 1 | 0 | 0 | 1 | 0 | Negative |
| 14-26 | 4279 | 3713 | 566 | −430 | 0 | 1 | 0 | 0 | 1 | 0 | Negative |
| 14-27 | 5016 | 3713 | 1303 | 307 | 0 | 1 | 0 | 0 | 1 | 0 | Negative |
| 14-28 | 5293 | 3713 | 1580 | 584 | 0 | 1 | 0 | 0 | 1 | 0 | Negative |
| 14-29 | 5176 | 3713 | 1463 | 467 | 0 | 1 | 0 | 0 | 1 | 0 | Negative |
| 14-30 | 5245 | 3713 | 1532 | 536 | 0 | 1 | 0 | [symbol] | 0 | 1 | Positive |
| 14-31 | 4681 | 3713 | 968 | −28 | 0 | 1 | 0 | 0 | 1 | 0 | Negative |
| 14-32 | 4769 | 3713 | 1056 | 60 | 0 | 1 | 0 | 0 | 1 | 0 | Negative |
| 14-33 | 5982 | 3713 | 2269 | 1273 | 1 | 0 | 1 | 0 | 1 | 0 | Negative |
| 16-1 | 3179 | 2198 | 981 | 182 | 0 | 1 | 0 | 0 | 1 | 0 | Negative |
| 16-2 | 2561 | 2198 | 363 | −436 | 0 | 1 | 0 | 0 | 1 | 0 | Negative |
| 16-3 | 3704 | 2198 | 1506 | 707 | 1 | 0 | 0 | 0 | 0 | 1 | Positive |
| 16-4 | 2981 | 2198 | 783 | −16 | 0 | 1 | 0 | 0 | 1 | 0 | Negative |
| 16-5 | 3352 | 2198 | 1154 | 355 | 0 | 1 | 0 | 0 | 1 | 0 | Negative |
| 16-6 | 4816 | 2198 | 2618 | 1819 | 1 | 0 | 0 | 0 | 0 | 1 | Positive |
| 16-7 | 3760 | 2198 | 1562 | 763 | 1 | 0 | 1 | 0 | 1 | 0 | Negative |
| 16-8 | 3452 | 2198 | 1254 | 455 | 0 | 1 | 0 | 0 | 1 | 0 | Negative |
| 16-9 | 2952 | 2198 | 754 | −45 | 0 | 1 | 0 | 0 | 1 | 0 | Negative |
| 16-10 | 3466 | 2198 | 1268 | 469 | 0 | 1 | 0 | 0 | 1 | 0 | Negative |
| 16-11 | 4256 | 2198 | 2058 | 1259 | 1 | 0 | 1 | 0 | 1 | 0 | Negative |
| 16-12 | 2843 | 2058 | 785 | −282 | 0 | 1 | 0 | 0 | 1 | 0 | Negative |
| 16-13 | 3719 | 2058 | 1661 | 594 | 0 | 1 | 0 | 0 | 1 | 0 | Negative |
| 16-14 | 3705 | 2058 | 1647 | 580 | 0 | 1 | 0 | 0 | 1 | 0 | Negative |
| 16-15 | 2908 | 2058 | 850 | −217 | 0 | 1 | 0 | 0 | 1 | 0 | Negative |
| 16-16 | 4794 | 2058 | 2736 | 1669 | 1 | 0 | 0 | 0 | 0 | 1 | Positive |
| 16-17 | 3438 | 2058 | 1380 | 313 | 0 | 1 | 0 | 0 | 1 | 0 | Negative |
| 16-18 | 3540 | 2058 | 1482 | 415 | 0 | 1 | 0 | 0 | 1 | 0 | Negative |
| 16-19 | 2844 | 2058 | 786 | −281 | 0 | 1 | 0 | 0 | 1 | 0 | Negative |
| 16-20 | 4508 | 2058 | 2450 | 1383 | 1 | 0 | 0 | 0 | 0 | 1 | Positive |
| 16-21 | 3120 | 2058 | 1062 | −5 | 0 | 1 | 0 | 0 | 1 | 0 | Negative |
| 16-22 | 2977 | 2058 | 919 | −148 | 0 | 1 | 0 | 0 | 1 | 0 | Negative |
| 16-23 | 3066 | 2188 | 878 | −68 | 0 | 1 | 0 | 0 | 1 | 0 | Negative |
| 16-24 | 3365 | 2188 | 1177 | 231 | 0 | 1 | 0 | 0 | 1 | 0 | Negative |
| 16-25 | 3226 | 2188 | 1038 | 92 | 0 | 1 | 0 | 0 | 1 | 0 | Negative |
| 16-26 | 3334 | 2188 | 1146 | 200 | 0 | 1 | 0 | 0 | 1 | 0 | Negative |
| 16-27 | 4593 | 2188 | 2405 | 1459 | 1 | 0 | 0 | 0 | 0 | 1 | Positive |
| 16-28 | 3421 | 2188 | 1233 | 287 | 0 | 1 | 0 | 0 | 1 | 0 | Negative |
| 16-29 | 4462 | 2188 | 2274 | 1328 | 1 | 0 | 0 | 0 | 0 | 1 | Positive |
| 16-30 | 2459 | 2188 | 271 | −675 | 0 | 1 | 0 | 0 | 1 | 0 | Negative |
| 16-31 | 3560 | 2188 | 1372 | 426 | 0 | 1 | 0 | 0 | 1 | 0 | Negative |
| 16-32 | 3396 | 2188 | 1208 | 262 | 0 | 1 | 0 | 0 | 1 | 0 | Negative |
| 16-33 | 3208 | 2188 | 1020 | 74 | 0 | 1 | 0 | 0 | 1 | 0 | Negative |
| 17-1 | 4080 | 3557 | 523 | 35 | 0 | 1 | 0 | 0 | 1 | 0 | Negative |
| 17-2 | 4182 | 3557 | 625 | 137 | 0 | 1 | 0 | 0 | 1 | 0 | Negative |
| 17-3 | 4164 | 3557 | 607 | 119 | 0 | 1 | 0 | 0 | 1 | 0 | Negative |
| 17-4 | 4014 | 3557 | 457 | −31 | 0 | 1 | 0 | 0 | 1 | 0 | Negative |
| 17-5 | 4020 | 3557 | 463 | −25 | 0 | 1 | 0 | 0 | 1 | 0 | Negative |
| 17-6 | 4331 | 3557 | 774 | 286 | 0 | 1 | 0 | 0 | 1 | 0 | Negative |
| 17-7 | 4581 | 3557 | 1024 | 536 | 1 | 0 | 0 | 0 | 0 | 1 | Positive |
| 17-8 | 3704 | 3557 | 147 | −341 | 0 | 1 | 0 | 0 | 1 | 0 | Negative |
| 17-9 | 3920 | 3557 | 363 | −125 | 0 | 1 | 0 | 0 | 1 | 0 | Negative |
| 17-10 | 4162 | 3557 | 605 | 117 | 0 | 1 | 0 | 0 | 1 | 0 | Negative |
| 17-11 | 4499 | 3557 | 942 | 454 | 1 | 0 | 0 | 0 | 0 | 1 | Positive |
| 17-12 | 5451 | 3510 | 1941 | 1195 | 1 | 0 | 0 | 0 | 0 | 1 | Positive |
| 17-13 | 4511 | 3510 | 1001 | 255 | 0 | 1 | 0 | 0 | 1 | 0 | Negative |
| 17-14 | 4088 | 3510 | 578 | −168 | 0 | 1 | 0 | 0 | 1 | 0 | Negative |
| 17-15 | 5142 | 3510 | 1632 | 886 | 1 | 0 | 0 | 0 | 0 | 1 | Positive |
| 17-16 | 4227 | 3510 | 717 | −29 | 0 | 1 | 0 | 0 | 1 | 0 | Negative |
| 17-17 | 4039 | 3510 | 529 | −217 | 0 | 1 | 0 | 0 | 1 | 0 | Negative |
| 17-18 | 4914 | 3510 | 1404 | 658 | 1 | 0 | 1 | 0 | 1 | 0 | Negative |
| 17-19 | 4515 | 3510 | 1005 | 259 | 0 | 1 | 0 | 0 | 1 | 0 | Negative |
| 17-20 | 4742 | 3510 | 1232 | 486 | 1 | 0 | 1 | 0 | 1 | 0 | Negative |
| 17-21 | 4222 | 3510 | 712 | −34 | 0 | 1 | 0 | 0 | 1 | 0 | Negative |
| 17-22 | 4396 | 3510 | 886 | 140 | 0 | 1 | 0 | 0 | 1 | 0 | Negative |
| 17-23 | 4273 | 3442 | 831 | 89 | 0 | 1 | 0 | 0 | 1 | 0 | Negative |
| 17-24 | 4674 | 3442 | 1232 | 490 | 1 | 0 | 1 | 0 | 1 | 0 | Negative |
| 17-25 | 4343 | 3442 | 901 | 159 | 0 | 1 | 0 | 0 | 1 | 0 | Negative |
| 17-26 | 4369 | 3442 | 927 | 185 | 0 | 1 | 0 | 0 | 1 | 0 | Negative |
| 17-27 | 4489 | 3442 | 1047 | 305 | 0 | 1 | 0 | 0 | 1 | 0 | Negative |
| 17-28 | 4716 | 3442 | 1274 | 532 | 1 | 0 | 0 | 0 | 0 | 1 | Positive |
| 17-29 | 4252 | 3442 | 810 | 68 | 0 | 1 | 0 | 0 | 1 | 0 | Negative |
| 17-30 | 3991 | 3442 | 549 | −193 | 0 | 1 | 0 | 0 | 1 | 0 | Negative |
| 17-31 | 4795 | 3442 | 1353 | 611 | 1 | 0 | 0 | 0 | 0 | 1 | Positive |
| 17-32 | 4826 | 3442 | 1384 | 642 | 1 | 0 | 1 | 0 | 1 | 0 | Negative |
| 17-33 | 4693 | 3442 | 1251 | 509 | 1 | 0 | 1 | 0 | 1 | 0 | Negative |
| | | | | | 29 | 72 | 13 | 2 | 82 | 17 | Totals |

| | |
|---|---|
| Sensitivity | 94 |
| Specificity | 84 |
| ROC Area | 0.94 |
| PPV | 55 |
| NPV | 99 |
| Obs | Observed sample numeric value |
| BG | Background numeric value between samples |
| RL | Relative luminosity (Obs − BG) |
| Delta NC | Value of the difference (Obs − NC) |
| NC | Pooled sera negative control value (not included in table) |
| Pos | Sample assignment is positive if > NC threshold |
| Neg | Sample assignment is negative if < NC threshold |

[symbol]

indicates data missing or illegible when filed

If the observed value for a sample is equal to the NC pooled reference, then the threshold is just the multiplier of standard deviations above the pooled reference for a given set of experimental conditions.

Threshold=X(SD)

Since the relationship between the average of several tested negative samples plus a multiplier of standard deviations for those samples tested relative to the pooled reference test sample is a constant, the threshold value can be automatically calculated by a simple formula as shown below.

{ObS_{NC-average}+X(SD)}/NC_{—}RL_{pooled reference}=Constant

### Therefore,

Threshold={Constant−1}NC_{—}RL

Threshold Factor={Constant−1}

A particular sample in a crib set is determined to be either positive or negative because its value is either above or below the threshold.

Positive Assignment

Obs_{relative luminosity}≧Threshold

Negative Assignment

Obs_{relative luminosity}<Threshold

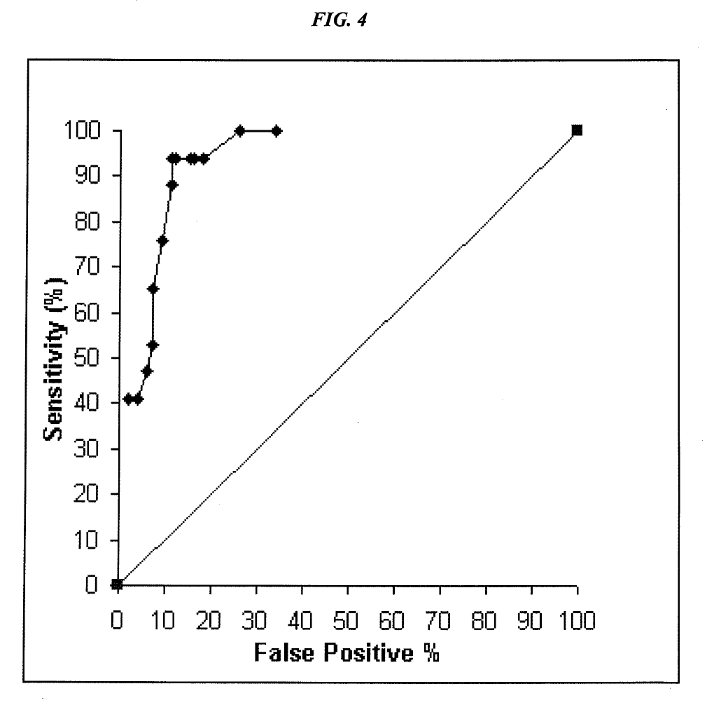

In practice the value for {Constant−1} is allowed to vary after testing so that the effect on Specificity and Sensitivity measurements can be determined. The standard ROC curve is then constructed (see, e.g., FIG. 4). From the table of values, the threshold factor is selected for any Specificity and Sensitivity cut off desired (Table 4). For example a threshold factor at 0.9 represents Specificity and Sensitivity values of 91 and 76% respectively.

TABLE 4
ROC Plot for 99 Samples Positive and Negative for M. Bovis.

| | | Threshold |
|---|---|---|
| Specificity (%) | Sensitivity (%) | Factor |
| 66 | 100 | 0.4 |
| 74 | 100 | 0.5 |
| 82 | 94 | 0.6 |
| 84 | 94 | 0.62 |
| 85 | 94 | 0.66 |
| 88 | 94 | 0.68 |
| 89 | 94 | 0.7 |
| 89 | 88 | 0.8 |
| 91 | 76 | 0.9 |
| 93 | 65 | 1 |
| 93 | 53 | 1.1 |
| 94 | 47 | 1.2 |
| 96 | 41 | 1.3 |
| 98 | 41 | 1.4 |

Normalizing Thresholds and Relative Luminosities

The calculated threshold is used to assign a sample based on its relative luminosity to either the positively infected or normal sera groups. If a threshold factor used results in 100% sensitivity, all of the positive samples should be identified (i.e., false negatives should be zero) but there may be a number of false positives (normal sera group) because the threshold is too low to eliminate their inclusion. The false positives can be eliminated in subsequent experiments with reflex supplemental testing.

By normalizing the relative luminosities against the selected threshold, a sample may be identified as either positive or negative because its value is either greater or less than 1.0 respectively. This is a more useful way of expressing values because it makes it easier to examine a list of samples to immediately see to which set the assignment should be allotted based on the numeric value for that sample.

The threshold is converted (normalized) from a relative luminosity value to an integer by dividing the observed relative luminosity for a sample by the threshold for that crib set. It can be appreciated at a glance when comparing one sample to the other that the ratio Delta/threshold is above or below 1.0. This alone determines the assignment for a positive or negative sample. Relative luminosity inspection by itself does not allow for such an easy interpretation of the assignment.

Positive Assignment

{Obs_{relative luminosity}}/Threshold≧1.0

Negative Assignment

{Obs_{relative luminosity}}/Threshold<1.0

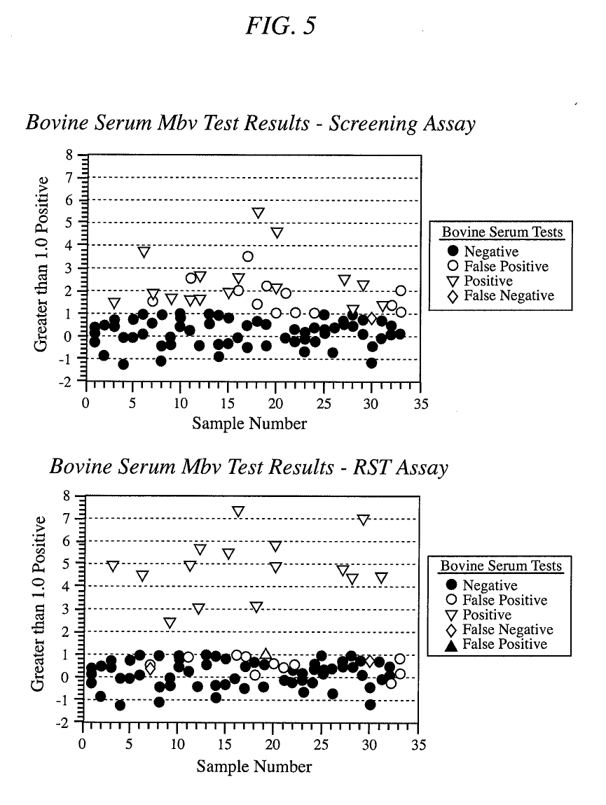

The automated and normalized values for the 99 tested animals are listed in Table 5, for the initial round of testing only. Graphic representations for screening and reflex supplemental testing are shown in FIG. 5.

By auto calculating the threshold, the criteria for sensitivity and specificity are set for a given set of experimental conditions. Each instrument will by the same criteria interpret results in an equivalent manner. And by normalizing the observed relative luminosity by dividing by the threshold calculated relative luminosity, all samples in a series or from instrument to instrument are easily assessed as either positive or negative for the threshold selected as they are either greater than or less than 1.0.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exemplary Protocols for Bovine Tuberculosis Testing

As discussed above, a capture probe comprising one or more peptide sequences or other antigens expressed in a target pathogen of interest may be used to bind to and detect the presence of anti-pathogen antibodies in blood, serum or other sample obtained from a subject. The skilled artisan will realize that the target to be detected is not limited, and any target against which circulating host antibodies are present may be detected using similar protocols. Conversely, antibodies against a target of interest may be used as capture probes and the presence of target antigen detected, for example, by sandwich ELISA or other techniques well known in the art.

### Materials and Methods

Production of Recombinant Antigen (RecAg) Probe

In the present exemplary embodiment of bovine tuberculosis detection, the capture probe used was a recombinant fusion protein expressed from two adjacent open reading frames (ORFs) within the Mycobacterium tuberculosis strain H37Rv genome, encoding CFP-10 and ESAT-6. Those two gene products were fused in frame by a process of PCR-directed mutagenesis in which the TGA stop codon of CFP-10 was changed to Gly (GGA) and an additional G residue was inserted downstream within the 32 nucleotide intervening region between CFP-10 and ESAT-6 in order to retain correct reading frame. This resulted in a fusion protein consisting of the complete CFP-10 gene product (100 amino acids) fused to the complete ESAT-6 gene product (95 amino acids) with an intervening 12 “nonsense” amino acids from the inter-ORF region. The recombinant antigen (RecAg) was then affixed to ferrite beads as a probe for bovine antibodies in infected animals.

CFP-10 Amino Acid Sequence

(SEQ ID NO:1)

MAEMKTDAATLAQEAGNFERISGDLKTQIDQVESTAGSLQGQWRGAAGTA
AQAAVVRFQEAANKQKQELDEISTNIRQAGVQYSRADEEQQQALSSQMGF
CFP-10 Nucleotide Sequence

(SEQ ID NO:2)

atggcagagatgaagaccgatgccgctaccctcgcgcaggaggcaggtaa
tttcgagcggatctccggcgacctgaaaacccagatcgaccaggtggagt
cgacggcaggttcgttgcagggccagtggcgcggcgcggcggggacggcc
gcccaggccgcggtggtgcgcttccaagaagcagccaataagcagaagca
ggaactcgacgagatctcgacgaatattcgtcaggccggcgtccaatact
cgagggccgacgaggagcagcagcaggcgctgtcctcgcaaatgggcttc
TGA
Inter-ORF region

(SEQ ID NO:3)

cccgctaatacgaaaagaaacggagcaaaaac
ESAT-6 Amino Acid Sequence

(SEQ ID NO:4)

MTEQQWNFAGIEAAASAIQGNVTSIHSLLDEGKQSLTKLAAAWGGSGSEA
YQGVQQKWDATATELNNALQNLARTISEAGQAMASTEGNVTGMFA
ESAT-6 Nucleotide Sequence

(SEQ ID NO:5)

atgacagagcagcagtggaatttcgcgggtatcgaggccgcggcaagcgc
aatccagggaaatgtcacgtccattcattccctccttgacgaggggaagc
agtccctgaccaagctcgcagcggcctggggcggtagcggttcggaggcg
taccagggtgtccagcaaaaatgggacgccacggctaccgagctgaacaa
cgcgctgcagaacctggcgcggacgatcagcgaagccggtcaggcaatgg
cttcgaccgaaggcaacgtcactgggatgttcgcatag
Entire Nucleotide Sequence

(SEQ ID NO:6)

atggcagagatgaagaccgatgccgctaccctcgcgcaggaggcaggtaa
tttcgagcggatctccggcgacctgaaaacccagatcgaccaggtggagt
cgacggcaggttcgttgcagggccagtggcgcggcgcggcggggacggcc
gcccaggccgcggtggtgcgcttccaagaagcagccaataagcagaagca
ggaactcgacgagatctcgacgaatattcgtcaggccggcgtccaatact
cgagggccgacgaggagcagcagcaggcgctgtcctcgcaaatgggcttc
GGAcccgctGaatacgaaaagaaacggagcaaaaacatgacagagcagca
gtggaatttcgcgggtatcgaggccgcggcaagcgcaatccagggaaatg
tcacgtccattcattccctccttgacgaggggaagcagtccctgaccaag
ctcgcagcggcctggggcggtagcggttcggaggcgtaccagggtgtcca
gcaaaaatgggacgccacggctaccgagctgaacaacgcgctgcagaacc
tggcgcggacgatcagcgaagccggtcaggcaatggcttcgaccgaaggc
aacgtcactgggatgttcgcatag

As indicated, the TGA stop codon at the 3′ end of the CFP-10 coding sequence was mutagenized to a glycine encoding GGA glycine codon. Also a G residue was inserted 6 bases downstream from the mutagenized GGA codon to make the fused ESAT-6 coding sequence in-frame with the CFP-10 sequence. Recombinant CFP-ESAT antigen was prepared by GenWay Biotech, Inc. (San Diego, Calif.).

Conjugation of Magnetic Beads to Fusion Protein

The CFP-110:ESAT-6 recombinant fusion protein antigen (CFP-ESAT) was conjugated to ferrite beads using a PolyLink Protein Coupling Kit for COOH Microspheres from Bangs Laboratories, Inc. (Fishers, Ind.) with slight modifications in the coupling process. Fusion protein free of Protein A and other interfering agents was covalently coupled to acrylic carboxyl (COOH) coated ferrite beads. Briefly, the carboxyl groups were activated with water-soluble carbodiimide with a NHS modification, which then reacts with these groups to create an active ester. The ester is reactive toward the primary amines on the protein to be conjugated to the beads. A representative protocol is disclosed in Data Sheet #644 (which accompanies the PolyLink Protein Coupling Kit).

The stock ferrite bead-CFP-ESAT conjugate, after blocking and washing in a glycinamide buffer, was suspended to a 2 mg/ml ferrite bead concentration in a standard dilution buffer with a microbial inhibitor. Prior to use, the beads were diluted to a concentration of 0.04 mg/ml in Standard Dilution Buffer (10 mM PBS, 0.1% BSA, 0.05% ProClin) for use in the bovine TB assay. Aliquots of the diluted bead preparation were used to spike diluted sera samples for each sample analyzed.

Materials

Carboxyl (COOH) coated ferrite microspheres and the PolyLink Protein Coupling Kit were from Bangs Laboratories, Inc., (Fishers Ind.). Biotinylated goat anti-bovine IgM (mu chain specific) was from KPL (Gaithersburg, Md.). HRP-SA solution, SuperSignal West Pico Luminol/Enhancer Solution and SuperSignal West Pico Stable Peroxide Solution were from Pierce (Rockford, Ill.).

Protocol

An exemplary step by step protocol for a rapid diagnostic test for Mbv Infection is provided below. More generally, in a typical test, a 1:50 dilution of a serum sample was made by adding 20 μl whole bovine serum (preferably azide-free) to 980 μl of Standard Dilution Buffer (10 mM PBS, 0.1% BSA, 0.05% ProClin) in a 1.5 ml Eppendorf Protein LoBind Tube. Then 280 μl of this sample was dispensed into a second Eppendorf LoBind tube and 70 μl of a well dispersed solution of 0.05 mg/ml ESAT-6/CFP-10 Bangs Biomag bead conjugate was pipetted into the bovine serum sample and incubated at RT for 15 minutes in a non-magnetic rack position.

The tube was then placed snugly into a magnetic rack and 720 μl of wash buffer (10 mM PBS, 0.05% Tween 20) was added to the sample, which was then incubated in the magnetic force field for an additional 2 minutes. With the sample tube remaining in the magnetic rack, all sample liquid was carefully removed by dropping an L-1000 pipette tip to bottom of tube and aspirating all of the buffer and sample (leaving behind two discrete dots of ferrite beads “snake-eyes” firmly affixed to the container walls in the force field). The tube was then removed from the rack and the beads were washed with 900 μl of a PBS-0.05% Tween wash buffer. Again the sample was placed in the force field for 2 minutes to affix beads. The wash step was again repeated so that 3 washes (the initial 720 μl spike and the 2 subsequent washes) were achieved. Then 250 μl of dialyzed KPL (Mu chain specific) biotinylated anti-bovine IgM (0.3 μg/ml in Standard Dilution Buffer) was used to wash beads from the sample wall back into solution. The beads were incubated at RT for 10 minutes with biotinylated antibody.

The sample was again spiked with 720 μl PBS-Tween wash buffer, incubated on magnets and washed as described before for the first sample incubation step. The tube containing only ferrite beads was then again removed from the magnetic force field and 200 μl of HRPSA (0.2 μg/ml in Standard Dilution Buffer) was used to disperse beads into solution where they incubate for 5 minutes at RT. Finally, using the identical process the sample was spiked with 720 μl PBS-Tween wash buffer and washed twice by dispersing and affixing beads at 2 minute intervals. Then 200 μl of a 1:1 mixture of luminal peroxide (Pierce) was added to each tube, uniformly dispersing beads into the solution.

The entire 200 μl luminol-peroxide-bead complex was transferred into a clear break-away well and deposited into a 12 sample crib (one sample was always a negative control sample consisting of a pooled collection of bovine sera which were tested negative for TB) for imaging. Emitted photons are captured for 10 minutes on the TOAD™ apparatus described above. Observed values for each sample, including negative control as well as background (determined as the average signal on 4 background fields) were then software processed with auto-threshold assignments. Results were determined by the program as Mbv negative or positive. Positives were retested in two Reflex rounds as described above. This general procedure may be followed or modified by routine experimentation according to techniques well known in the art. A more detailed exemplary step-by-step protocol of use in the claimed methods is described below.

Detailed Protocol

The skilled artisan will realize that the detailed protocol steps listed below represent a preferred embodiment of the claimed methods and that various steps, concentrations, amounts, times, etc. may be varied by routine experimentation within the scope of the claimed methods. All serum dilutions should preferably be made and all testing conducted in 1.5 ml Eppendorf Protein LoBind tubes. One negative control sample was included in each (12 sample) crib set. A 280 μl aliquot of a pooled collection of bovine sera (1:50 dilution) which have tested negative for TB was used in place of an individual serum sample and processed exactly the same as the bovine samples being tested.

Inclusion of a positive control is optional, but it is a good idea to run periodically for quality control testing of assay reagents. Fifty μl of a solution of 0.04 mg/ml BBSA conjugated Bangs Biomag beads made up in Standard Dilution Buffer (10 mM PBS, 0.1% BSA, 0.05% ProClin) was dispensed into a 1.5 ml Eppendorf Protein LoBind tube. An additional 280 μl of Dilution Buffer was added to bring up volume for adherence of beads to magnets. The positive control was processed exactly like the other samples starting at Step 6 below (wash steps).
1) Make a 1:50 dilution of each serum sample to be tested by adding 20 μl whole bovine serum (without Na azide) to 980 μl of Dilution Buffer (10 mM PBS, 0.1% BSA, 0.05% ProClin) and aliquot 280 μl into an a 1.5 ml Eppendorf Protein LoBind tube.
2) Pipette 70 μl of a well dispersed solution of 0.05 mg/ml ESAT-6/CFP-10 Bangs Biomag bead conjugate into the 280 μl bovine serum sample and incubate at RT for 15 minutes. Place each tube snugly in a magnetic well and spike sample with 720 μl Wash Buffer (10 mM PBS, 0.05% Tween 20). Incubate on magnets for an additional 2 minutes to allow ‘snake eyes’, condensed patches of ferrite beads, to form on sides of tube exposed to magnets.
3) Carefully aspirate liquid from the tube without disturbing beads bound to the sides by placing an L-1000 pipette tip into the tube and suctioning fluid from bottom in a smooth, steady stream until air bubbles are observed in the tip. (One pipette tip was dedicated to one sample throughout the entire protocol).
4) After removing tube from magnetic wells, add 900 μl of Wash Buffer and gently mix beads back into solution by slowly pipetting solution in and out of the tip against the wall of tube (taking care to minimize foam production). Place tube back in magnetic well and incubate for 2 minutes. Pipette off fluid without disturbing bound beads as described above. Repeat this step 1 more time for a total of 3 washes (the initial spike and 2 subsequent washes).
5) After removing tube from magnetic well, add 250 μl of dialyzed KPL biotin-conjugated affinity purified goat anti-bovine IgM (mu chain specific) antibody prepared at 0.3 μg/ml in Dilution Buffer to each sample and disperse beads by gently hand vortexing into solution. Incubate at RT for 10 minutes, place snugly in magnetic wells, spike with 720 μl Wash Buffer and incubate for an additional 2 minutes.
6) Carefully pipette off liquid as in Step 3 with an additional 2 wash steps as described previously in Step 4. Remove sample tube(s) from magnetic wells.
7) Add 200 μl of Pierce HRP-SA (prepared at 0.2 μg/ml in Dilution Buffer) to each tube, disperse beads into solution and incubate RT for 5 minutes. Place snugly in magnetic wells, spike sample with 720 μl Wash Buffer and incubate for 2 minutes at RT
8) Carefully pipette off liquid and wash 2 times more with as described previously in step 4. Remove sample tube(s) from magnetic wells.
9) Prepare a solution of Pierce SuperSignal West Pico Luminol Enhancer and SuperSignal West Pico Stable Peroxide in a 1:1 ratio and add 200 μl to each sample tube. Vortex the contents of each tube gently by hand and squirt liquid down sides of tube with a L-200 pipette tip to disperse beads into solution. Transfer the entire 200 μl volume from each sample tube into a clear plastic breakaway well and deposit into a 12 sample crib (one sample of which is a negative control (1:50 dilution) of pooled Mbov negative sera).
10) Emitted photons are captured for 10 minutes on the TOAD™ apparatus described above and with analyzed with software under optimization conditions using a cooled CCD high resolution camera. Observed values for each sample and negative control as well as background (determined as the average of 4 background fields) are inserted into the Reflex-Testing—Automatic Threshold Adjustment Excel Sheet template. Results for each sample are analyzed by an algorithm in the excel template and given the designation of Mbov negative or positive. Positives in this first round of screening are retested exactly as described above (Reflex Test #1). Positives in this second round of testing are tested one final time (Reflex Test #2) for confirmation of positive status (M. bovis infection).

The exemplary protocol discussed above is designed for optical detection with samples that spontaneously emit light, using any known chemiluminescent or bioluminescent detection system. In this case, no external source of excitatory light is needed to detect bound analytes. The skilled artisan will realize that alternative systems may be utilized, such as fluorescently tagged detection molecules that bind to target analytes. Such systems are well known in the art. The use of a fluorescent detection system would necessitate additional components, such as an excitatory light source (e.g. laser, photodiode array, etc.), cutoff filters to screen the photodetector from excitatory light, and other such known components for fluorescent detection systems. The illustrative embodiment discussed above possesses the advantages of simplicity of construction and use, low cost, and sensitive detection of target analytes with minimal background noise.

### Results

Results from three blinded Sensitivity and Specificity Studies obtained using the Rapid Diagnostic Test and Reflex Supplemental testing were pooled into one 99 sample study. Results from the initial screen are shown in Table 5. Assays were performed as described above, with Ferrite conjugate GW ESAT 70 ul, 0.04 mg/ml probe, 10 mM PBS 0.05% Tween 20 wash buffer, IgM 0.3 ug/ml 10 mM PBS 0.1% BSA 0.05% PC antibody solution, 0.2 ug/ml HRPSA, 10 mM PBS 0.1% BSA 0.05% PC, and 15, 10, 5 minute incubation cycles. The three studies exhibited an average sensitivity of 88, specificity 84, ROC 0.94, PPV 52 and NPV 96. A representative image for a single crib set of 11 sera and the negative control is shown in FIG. 3.

The advantages of reflex supplemental testing are shown in Table 5. It is clear that the reflex supplemental testing procedure decreases the number of false positive results obtained. More than 500 tests on sera have been completed. Approximately 20 positive samples and more negative sera samples have been used in a 1:50 dilution to identify factors that influence the effectiveness of the assay. Certain of these factors are discussed below.

Temperature Effects on Mbv Detection

The effect of temperature on the assay can significantly alter outcomes. Both lower temperature (˜4 C) and higher temperature (˜37 C) make it much more difficult to differentiate positive and negative serum samples from one another. At higher temperatures all samples including negative sera appear to react very strongly so that so much light was produced that the signals are essentially indistinguishable. Lowering the temperature has quite the opposite effect with signal loss for both negative and positive sera. The preferred temperature appears to be very near room temperature (˜25 C).

pH Effects on Mbv Detection

Based on limited analysis, optimal signals were detected at pH˜7.4. Lowering the pH to 4.0 resulted in a marked decrease in signal for positive sera.

Biotinylated Secondary Antibody

Many secondary biotinylated antibodies have been tested in the assay including biotinylated IgY anti-bovine IgG, biotinylated goat IgG anti bovine IgG and biotinylated goat IgG anti-bovine IgM. Combinations have also been tested. All individual and combination tests have been successful in differentiating positive and negative sera to varying degrees, but the simplest and currently optimized antibody to use is biotinylated goat IgG anti-bovine IgM. The optimal concentration with 0.2 ug/ml HRP_SA was 0.3 ug/ml IgM in a standard dilution buffer.

Protein A Interference

Much effort was directed to prepare ferrite bead conjugates free of Protein A. Protein A was found to substantially interfere with assay development in generating false positive results and/or high background. Even trace amounts of Protein A present in solvent or substrate during RecAg conjugation resulted in beads that strongly react with biotinylated antibodies and bovine serum antibodies. With trace contamination the background signal for a negative control will be as bright or brighter than a positive serum test.

Protein A is commonly used for antibody purification schemes and is often co-eluted with the antibody and must be eliminated or false positives signals will be observed. This is true regardless of how the antibody will be used because Protein A binds to both Fc and Fab (to a lesser extent) allowing for bridging to other antibodies.

If the antibody is to be conjugated to ferrite beads and has Protein A in the mix, some of the Protein A will end up on the ferrite bead along with the antibody. Since we detect the antibody response on the ferrite as an antibody-antigen interaction typically with a biotinylated antibody against that same or linked target that the primary antibody has recognized, any biotin picked up (by the HRPSA reaction with luminol) would be assumed to be a confirmation that the primary antibody had picked up epitope. But with Protein A on the ferrite bead and a sensitive detector, the Protein A catches some of the biotinylated secondary antibody resulting in light detection at the end of the assay regardless of what happened to the primary antibody. A false positive is detected in the absence of epitope.

If the antibody is biotinylated (intended as a secondary antibody detector) and Protein A is present, some of the Protein A is biotinylated and because it binds with the Fc and Fab of the biotinylated antibody, ends up in the reagent even after dialysis. Because the Protein A can bridge between two antibodies linking them, even if no epitope is present the biotinylated antibody will be linked to the antibody on ferrites (or spotted on nitrocellulose) again resulting in a false positive detection.

Antibody Dialysis to Remove Protein A

Protein A's affinity for antibodies is dramatically reduced at lower pH. We use a 10 mM Glycine pH 2.8 dialysis and drive the dissociation by placing the antibody in a dialysis tube with 100 kD membrane cut-off and add to the Glycine a cationic resin to trap protein passing the membrane in the dialysate. Typically 1 gram of a strong cationic exchange resin is added to the Glycine pH 2.8, 1 liter exchange against 5 ml of antibody in the membrane tube (floatalyzer) and dialysis is performed 16 to 24 hours with 2 to 3 liter exchanges. The dialysate is switched to pH 7.4 10 mM PBS liter and continue dialysis an additional 16 to 24 hours with 2 to 3 liter exchanges. So the ratio is 200:1×3 in the Glycine buffer pH 2.8, and the same in the PBS media (million fold first and million fold second exchange ratios). If working with a biotinylated antibody, a 10 mM PBS 0.1% BSA 0.05% proclin (antimicrobial inhibitor) solution is used for the second set of exchanges. This results in very pure antibodies, in the first example in PBS ready for ferrite bead conjugation or biotinylation, and in the second example ready for dilution to an appropriate level for assay work.

Removal of Protein A from Proteins Between 10 kD and 100 kD in Size

Proteins in the category are usually epitopes. The immunoassay will not work properly if Protein A is present, as it will bind non-specifically to antibodies, and the epitopes are assumed to be contaminated with Protein A. An antibody is used to bind Protein A at a pH where the association is strong. The first step is to spike the protein solution with an appropriate IgG antibody, preferably free of biotin. It should also be from a different family than the antibody that will be used in the assay generally. The antibody is incubated 30 minutes with the protein solution, typically in a PBS pH 7.4 solution. Then filtered and the filtrate collected using a 100 kD membrane filter (e.g. Amicon 100 kD filters). The Protein A at 47 kD would normally pass the filter, but will not pass with the antibody to which it is bound. The protein of interest passes this filter and is collected, free of Protein A. The collected protein is dialyzed against PBS pH 7.4.

A typical example would be CFP10ESAT6 which is about 27 kD. A solution of 500 uG/ml of the fusion protein 1 ml volume is spiked with 50 uL of Goat antimouse IgG antibody 0.7 uG/ml and allowed to incubate 30 minutes. The solution is transferred to an Amicon 100 kD filter, placed on the centrifuge and filtrate collected. Recovery filtrate volume will be about 1 ml. The filtrate is transferred to a 1 ml dialysis tube 10 kD membrane and placed in a 1 L 10 mM PBS pH 7.4 solution and dialyzed with 3 exchanges over 24 hours. The freshly isolated fusion protein is then conjugated to ferrite beads in a modified protocol.

Removal of Protein A from Buffers and Other Reagents

Many commercial reagents are contaminated with Protein A. All reagents are filtered through a 30 kD Amicon filter before activating conjugation steps to be certain that Protein A does not inadvertently get back into the mix at these points. For example, in the MES washing steps of the ferrite beads before conjugation, and in preparing EDC and NHS reagents for bead activation the MES is pre-filtered. Filtered solutions or clean solutions are also used for quenching and then stabilizing the beads

Variability in Sera Samples

One difficulty in developing the Mbv assay concerned marked variability in sample analysis. Even when the same sample was repeatedly tested, considerable variance in the relative luminosity compared to a pool of negative controls was frequently observed. Many technical modifications and method improvements were initiated to minimize the variance issue but still there was poor reproducibility in terms of an absolute RL value for any given sample. However it was ultimately appreciated that the negative sera and positive sera behave differently in as far as positive sera are persistently well above the negative pool of sera used as a negative control, while the negative sera that test high do not consistently test high and can be eliminated by reflex supplemental testing.

In general it was found that very reproducible outcomes were achieved when a threshold for positive sera was set at approximately 1.6 standard deviations above the average for multiple negative sera tested. At this threshold in a simple, non-reflex screening the sensitivity was 88% and specificity 84%. All of the 99 samples were tested using randomization and blinding protocols to avoid any possible bias in the analysis. Some of the positive sera appeared very weakly positive, but were nevertheless detected both in the initial screen and on supplemental reflex testing. A comparison between initial screening and supplemental reflex testing results is shown in Table 5. It is important to appreciate that in very low prevalent states any test with less than 100% specificity will result in many false positives rendering the positive predictive value nearly useless. By adopting a reflex supplemental testing strategy, with each pass the pool of animals tested was enriched and concentrated providing the sensitivity was also high. This makes it possible to eliminate false positives with confidence in identifying truly infected animals.

Some of the determinants that optimized the assay relate to the use of 0.05% Tween in the wash solution and use of Lobind eppendorf tubes. Very low but unpredictable amounts of absorption onto the surface of the plastic containers contributed to variability and this could be reduced in the Lobind format. Considerable effort was directed to find evidence for possible bead loss but it appears that this was not a significant factor in the variance observed.

### ROC Curves

The data may also be analyzed by another statistical application called the ROC (Receiver Operating Characteristic) curve, which takes into account variation in Sensitivity and Specificity as the arbitrary test threshold position is varied. How useful a test is at discriminating between two populations of true negatives and true positives is characterized by examining the area under the ROC curve. The closer the area is to 0.5, the worse the test and the closer it is to 1.0, the better the test. The area under the ROC curve after one initial screening using the Rapid Diagnostic test disclosed herein is 0.94 (FIG. 4).

### Positive Predictive Value and Prevalence

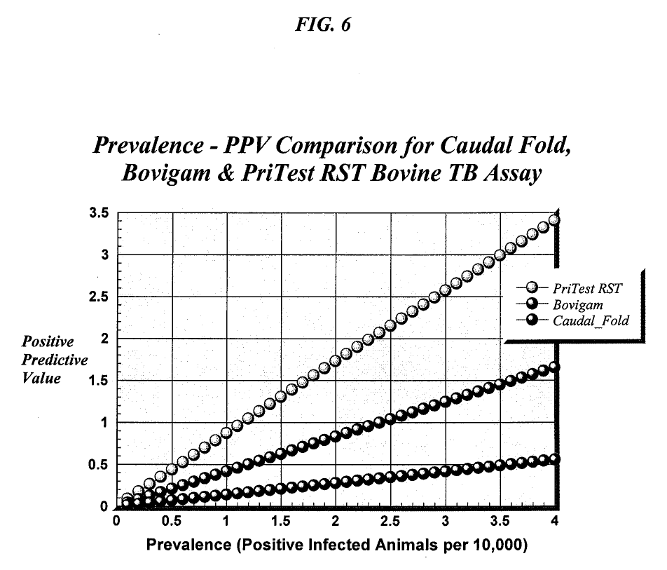

Using the reported Sensitivity and Specificity values for Caudal Fold, Bovigam, and the presently disclosed assay results in 99 randomized cattle sera tested using reflex supplemental testing, a direct comparison of Positive Predicted Values (PPV) based on the method used is shown below (FIG. 6). The test with the higher PPV for a given prevalence will predict more precisely which animals are infected. Positive Predicted Value is calculated by the formula;

$$\frac{\begin{matrix}Total True Positive Sera in Group - \\ Non Detected True Positives \end{matrix}}{Total Positives Detected in the Study}$$

The superiority of the presently claimed methods over existing assays—the Bovigam and Caudal fold—in positive predictive value is apparent from FIG. 6. With reflex supplemental testing, because of the high specificity (99%) it is anticipated that most of the false positives expected in a low prevalent testing environment will be released, rather than incorrectly slaughtered, as indicated by the reduction in number of false positive results with the reflex supplemental testing method disclosed herein.

### CONCLUSIONS

A rapid method of differentiating between positively infected cattle with Mycobacterium bovis and uninfected cattle using a recombinant is disclosed herein. Screening of 99 samples yielded a sensitivity of 88% and specificity of 84%. By adjusting the threshold the test can be set to capture 100% of positively infected animals with a specificity of about 74%.

Reflex supplemental testing results in an improved outcome by reducing or eliminating false positive results. It requires only a minimal number of additional tests to increase the specificity to 99% without any change in the sensitivity (88%). Based on PPV, the disclosed reflex supplemental testing method is superior to either the Bovigam or Caudal fold test, which are the most accurate tests presently known in the art. Because it is faster and far less expensive and not technically demanding to conduct, it is a useful tool for eradicating M. bovis in cattle.

Serum is the only sample needed so a single visit and sample can provide the needed information to determine if the animal is infected. Serum can be stored frozen for an indefinite period of time until ready to commence testing. Repeat testing can be done as is deemed appropriate without any concern of having altered the animal's immune response in the sampling of blood.

### Example 2

### Method of Detecting Pathogens

The present methods, compositions and apparatus allow the effective and rapid identification and isolation of tuberculosis infected animals, using a CP10_ESAT fusion protein assay. In such an assay, a synthetic protein or peptide, displaying one or more antigenic epitopes of Mycobacterium bovis, is allowed to react with blood, serum or plasma from an animal suspected of being infected with M. bovis. Binding of antibody to the target antigen indicates that the animal is infected with M. bovis. As the CP10_ESAT fusion protein used displays immune cross-reactivity with Mycobacterium species known to infect other species, such as humans, the skilled artisan will realize that the same compositions, apparatus and methods may be used to detect tuberculosis in other species, such as humans, bison, deer or any other animal known to be a potential carrier for Mycobacterium sp. Proteins homologous to CP10 and ESAT are well known antigens for various Mycobacterium species (e.g., van Pittius et al., Genome Biology 2(10), 2001). With the CP10_ESAT fusion protein attached to a substrate, such as a protein chip, Grabber™ slides or magnetic beads, the anti-Mycobacterium antibodies present in infected host blood will also bind to the substrate. After appropriate wash steps, the presence of anti-Mycobacterium antibodies in a sample may be detected, for example, by addition of commercially available biotinylated anti-bovine antibodies, followed by chemiluminescent assay using conjugated HRP-SA, peroxide and luminol as discussed above.

A preliminary set of 33 samples of bovine serum were tested in a blinded study, using the protocols disclosed above. Under the conditions of the assay, there were zero false negative results. Out of 33 blinded samples, there were 4 false positive results obtained on the first round of assays. Each sample that tested positive on the first round was subjected to reflex retesting, using the same assay at higher sensitivity. After reflex retesting the number of false positives declined to zero. An ROC curve analyzing sensitivity vs. specificity illustrates that the assay can obtain essentially 100% sensitivity at a specificity of about 70%, while 100% specificity is obtained at a sensitivity of about 70% (see, e.g., FIG. 4). By performing the reflex sample testing at 100% sensitivity and 70% specificity, it is possible to eliminate virtually all false positive test results.

The skilled artisan will realize that the reflex supplemental testing method disclosed herein is not limited to detection of tuberculosis, but may be used to detect virtually any type of pathogen, contaminant, biohazard, biowarfare agent, diseased cell or other condition, so long as an appropriate ligand and/or probe may be obtained. In this particular exemplary embodiment, the method discloses performing supplemental reflex testing on all positive samples, using the same assay at conditions of about 100% sensitivity and about 70% specificity. Under these conditions, there are virtually no false negative results. Therefore, each round of iterative reflex testing will eliminate about 70% of true negative subjects from the positive test pool. Use of 4 rounds of iterative reflex testing, applied to only those samples testing positive on the previous round, should result in a close to 100% accuracy in detecting infected animals. For example, 4 cycles of iterative testing at a 70% specificity would result in a false positive rate of 0.3×0.3×0.3×0.3, or less than 1%. Use of 5 cycles would result in about a 0.25% false positive rate. Use of 6 cycles would result in an false positive rate of less than 0.1%. Further, because the reflex testing is only performed on positive samples, the number of assays performed diminishes rapidly with each cycle, resulting in a rapid, efficient and inexpensive method to achieve close to 100% testing accuracy. The reflex supplemental testing strategy provides within a few hours a clear path to infected animals and they never re-enter the herd to infect others.

In the developed countries, such as the US and Europe, the percentage of infected animals in a herd is relatively low. Assuming an infection rate of 1 in 1,000,

| Event | Observations |
|---|---|
| 1000 Cattle possibly 1 infected with MB | 1 MB infected animal |
| | 999 non-infected animals |
| Test Results for 98% Sensitivity and | 20 “false positive” |
| Specificity | 49 of every 50 true positives |
| | detected |
| Expected Outcome | 21 animals all test positive |
| | Must use confirmatory tests to |
| | find the one animal infected, |
| | assuming he is not back in the |
| | herd infecting others. |

Suppose an assay was used that in the laboratory setting could consistently produce a 98% Sensitivity and 98% Specificity. That would by most standards be considered quite good. But in the above example where many negative animals are screened to find a single infected animal (1 in 1000 animals infected), we would expect 20 of the non-infected animals to test false positive. We see that high Sensitivity and Specificity alone will not do the job. We would expect to measure 21 animals positive in that group, assuming the infected animal didn't slip quietly back in with the herd undetected as a false negative. One in every 50 positive animals would escape to re-infect the herd with a 98% Sensitivity. We don't know how to identify which one of the 21 is truly infected. So a far more expensive confirmatory test must then be done on these 21 animals to settle the issue.

If the herd size is 10,000 with a single infected animal, the first pass should provide 201 positives. And as the number of infected animals in the herd increase, a number of false negative test results will occur. Undetected animals will re-enter to infect others in the herd. No single test will ever consistently produce the level of Sensitivity and Specificity required to effectively isolate infected animals with 100% certainty. Reflex supplemental testing involves testing deliberately at a threshold where false positives are expected because the threshold for detecting positives has been set sufficiently low to catch 100% of positively infected animals. By then testing in a second batch all of those animals that did not pass negative on the first run again in the same assay, a second batch of truly non-infected animals can be safely released. The process can continue in sequential steps until only the true positive isolates are confirmed and rapidly centers on infected animals. This method is in principle much more effectively than a single step assay.

If the Specificity is moderately high at the threshold where 100% Sensitivity can be expected, then large numbers of animals are released in each run. A single tube of sera is more than adequate and tests are only repeated on “positive” outcome animals. Because Sensitivity is 100%, an animal that tested positive in the first run and negative in the second or third run can be safely released as truly un-infected. The disclosed exemplary assay provides 70% Specificity at the threshold for 100% Sensitivity. We can calculate the numbers to get to our positive infected animal in the 1000 herd number. By illustration the calculations are shown for a 10,000 animal number.

| | Observations | |
|---|---|---|
| | Infected or | Released as |
| Reflex Supplemental Testing | “Positive” | “Negative” |
| 1000 Cattle possibly 1 | 1 MB infected | 999 non-infected |
| infected with MB | animal | |
| Initial Test Screen | 300 | 700 |
| Reflex # 1 | 90 | 210 |
| Reflex # 2 | 27 | 63 |
| Reflex # 3 | 8 | 19 |
| Reflex # 4 | 2 | 6 |
| Totals | 427 | 998 test non- |
| | | infected |

The sera for each animal is retained and reflex tested only if it is positive in a test. The total number of tests to isolate 2 animals out of a herd size of 1000 is 1128 and less than 1 ml of sera for each animal is more than sufficient to complete all tests. Because so many animals are released with each pass, only 128 additional tests are required to isolate the infected animal to one of two possibilities. To screen and cull to 2 animals in a herd size of 10,000 it would require only 6 Reflex test procedures and 11,285 tests. We would expect to release 7,000 animals in the initial screen.

### Example 3

### Methods and Protocols for Ferrite-MPB83 Assays Ferrite Vinyl Amine Conjugates & MPB83 Conjugates

Vinyl amine coated ferrites are manufactured by conjugating commercially provided carboxyl ferrites to vinyl amine. The coated ferrites are then covalently linked with a target antigen that will be used in the assay. The conjugation efficiency is very high and minimizes false positive interactions between negative serum samples, that are commonly experienced with non-coated carboxyl ferrites.

The following protocol has been used successfully for MPB83 and for K39 antigen conjugates. We use the acronym FCXPT for carboxyl ferrites EDC conjugated to poly vinyl amine polymer that has then been activated to accept antigen conjugates. We use the sulfo (SIAB) Pierce method to link antigen to sulfhydryl groups on the ferrites to produce the final product FCXPTSM (MPB83 conjugated ferrites) that are then oxidized to eliminate electron donors that could interfere with horse radish peroxidase luminol peroxide reactions we expect for detection of a positive reaction with ferrites.

Pierce and Poly Science recommend that ideal ferrite conjugation with EDC occurs at pH 5.5 and is complete in a few hours. We have found the reaction is far more efficient at pH 4.0 and in the conjugation of vinyl amine with activated carboxyl ferrites takes more then 24 hours for ideal conjugation.

Materials and Reagents
PVAm, Lupamin BASF product # 5095-15 ml
MES Pierce Product # 28390, buffer 50 mM, pH˜4 with WFL non filtered water ˜500 ml
Borate Buffer pH 8.2 50 mM WFL water non filtered ˜500 ml
EDC Fluka product # 03450
NHS Pierce product # 24500
Sulfo SIAB Pierce product # 22329
Traut's reagent Pierce product # 26101
Carboxyl ferrites 21.4 mG/ml (FC) Poly Sciences product # BP 618
50 ml conical tubes rinsed with distilled water and then MES
Concentrated HCL
MPB83 lyophilized 1 mG Lionex Diagnostics Product code MB-MPB83
2 Conical flasks
Axygen tubes
Lobind tubes
50 ml conical magnetic collector cage
1M Hydroxylamine in Borate Buffer pH 8.2
Magnetic cage—neodymium 3 column collector used to efficiently hold ferrites in place so that reagents can be exchanged without loss of ferrites.

Preparing PVAm and Activated Carboxyl Ferrites (FCXPT)

Rinse 2 conical flasks first with water, then with MES buffer and shake dry. Include the caps with the rinsing procedure. One tube will be used to prepare PVAm for conjugation to the activated ferrites, and the second tube will be used to activate ferrites. They will then be mixed once ferrites are activated.

Activation of Ferrites
EDC—23 mg weighed into dry Axygen tube and sealed
NHS—42 mg weighed into dry Axygen tube and sealed
Add 30 ml MES to one of the 50 ml conical tubes and label it EDC/NHS/FC
Wash 300 μl (21.4 μg/μl) in a Lobind tube with 1 ml MES buffer 3× using magnetic port to collect ferrites with 4 minutes between each collection discarding MES after each wash.
Add the EDC and the NHS to the 50 ml conical washing the Axygen tubes with MES from the 30 ml, transferring all contents to the 50 ml conical.
Suspend the washed FC in this MES using a 1 ml pipette, and transfer the contents to the 50 ml conical being careful to wash all ferrites into the 30 ml MES mix, and then discard the Lobind.
Allow this reaction to proceed for 60 minutes (timer).

PVAm Preparation for conjugation
While the FC is activating, place 15 ml of PVAm in the 2^{nd} conical.
Adjust the pH with concentrated HCl to 4.0
Mix and recheck the pH until you are satisfied it is correct.

First Conjugation Step FCXP
After 60 minutes FC activation, add the entire contents of the pH adjusted PVAm (˜15 ml) to the 30 ml MES conical.
Wash the PVAm conical walls with the solution to be certain that all material has been transferred to a single 50 ml conical
Adjust the volume to 50 ml
Place the conical on the rotor and let it react 24 hours.

Conversion of Amines to SH & MPB83 Conjugation (FCXPT).

The second step conjugation converts primary amines to sulfhydryl groups to then conjugate activated MPB83 to the ferrites. This reaction takes place in a borate buffer and it will be necessary to collect your ferrites that are in MES, quench them, and proceed in the borate buffer reaction. This will involve several wash steps, and each takes time, especially in the first step because ferrites carry a high positive charge. The times shown have been worked out by careful measurements, checking with a small aliquot to see if in an Axygen tube snake eyes could be seen on our processing plate, adding the contents back if collected ferrites are observed and waiting longer if they are seen.

Activation MPB83 SSIAB
Initiate MPB83 activation.
Weigh in a dry Axygen tube 3 mG SSIAB and seal
Open 2 500 uG vials of MPB83.
Add 400 uL borate buffer to solubilize the MPB83, and transfer the entire contents to the Axygen tube with SSIAB.
Wash each vial with an additional 400 uL of borate buffer transferring the contents to the Axygen tube with SSIAB (total volume 1600 uL
Place the Axygen tube on the rotor, light protected and allow to react while you work with the ferrites.

Collect and Quench Ferrites:
Place the 50 ml conical with ferrites in the magnetic cage and wait 2.5 hours to allow all ferrites to move to the wall. They move slowly and pack slowly because of the charge on amines at this pH in MES.
Discard the MES by pouring the contents in to a waste container while the tube is in the cage.
Remove the tube from the cage.
Add ˜50 ml borate buffer to the ferrites, and 2 ml 1M hydroxylamine.
Suspend ferrites completely using auto pipette washings on the wall so all ferrites are suspended.
Place the borate quench solution with ferrites in the cage.
Collect ferrites 30 minutes. (wash #1), and then discard the borate buffer by pouring the contents into a waste container while the tube is in the cage.
Repeat this procedure filling this time with borate buffer, dispersing ferrites, and collecting in the cage for wash # 2, and again for wash # 3, giving 30 minutes for each ferrite magnetic collection.

Traut's Activation Step:
Discard the borate from the 3rd wash step, pouring it into a waste container while the tube is in the cage.
Fill the tube with 50 ml borate buffer, dispersing ferrites again.
Weigh out 42 mG Traut's reagent and add it to the ferrites that are dispersed.
Place the tube on the rotor and allow it to react 1 hour.

Collect Traut Activated Ferrites.
After 1 hour on the rotor, place the ferrite tube with Traut activated ferrites in the magnetic cage.
Collect ferrites 30 minutes, and then discard spent reagents by pouring the contents into a waste container while the tube is in the cage.
Fill the tube with 50 ml borate buffer, collect 30 minutes on the cage, and discard borate buffer as before (wash #1), and repeat again with another wash #2.
Fill the tube to the 50 ml mark with borate buffer.
The concentration of ferrites FCXPT is 128.4 μg/ml

Conjugate MPB83 to Ferrites:
Calculate the volume of Traut activated ferrites to equal 3 mg of ferrites. In this protocol, it is 23.4 ml.
Transfer 23.4 ml FCXPT to a fresh washed 50 ml conical
Add the SSIAB activated MPB83 Axygen tube contents to this 50 ml conical and bring the volume to exactly 50 ml.
Place the tube on the rotor and let the reaction proceed 24 hours with light protection.
After 24 hours, the reaction is at an end point and you have 50 ml of 60 μg/ml FCXPTSM ferrites.

Oxidation of Ferrites

We have observed that ferrites are much stabilized and give a better signal if they are oxidized before they are transferred to standard dilution buffer for storage and subsequent use. It appears that sulfhydryl groups and possibly other groups associated with the antigen are capable of competing with hydrogen peroxide as an electron donor, rendering the subsequent luminol peroxide light reaction less intense unless the ferrites are first oxidized to eliminate the quenching that occurs. Without this step, true positive reactions are not easily differentiated from negative reactions unless they are very intense.

Ferrite oxidation is accomplished by allowing the ferrites to react at least 30 minutes with a 50% diluted Super Signal West Pico Peroxide Solution (Pierce 1859674). This is done in the 50 ml conical with the just prepared 3000 μg of ferrites by collecting the ferrites first, then discarding 25 ml of the solvent and adding to this 25 ml of peroxide solution and re-suspending the ferrites. After 30 minutes, the ferrites are again collected, the spent peroxide solution is discarded, and the ferrites are suspended in standard dilution buffer. They are stable for at least 6 months and ready for use in the assay.

### Example 4

### UK Badger and Cattle TB Test Results

Despite many technological advances in the field of diagnostics, to date there are no rapid inexpensive and accurate means to diagnose even minimally active mycobacterial infections. No single test stands above the others and they are all generally poor, failing in either sensitivity or specificity. Zahrani et. al. (Am. J. Respir. Crit. Care Med., Volume 162, Number 4, 2000, 1323-1329) report typical findings for diagnosis in 60 cases of active TB. Sensitivity and specificity, respectively, of the following tests are mycobacterial culture 73% and 100%; PCR 42% and 100%; chest X-ray 67-77% and 66-76%; tuberculin testing 94% and 20%; and serology 33% and 87%. The tuberculin test is the only test currently able to detect latent disease, but there are many false positive tests by this method.

Although there has been a long and persistent search for serological assays which can detect circulating antibody to mycobacterial infections, results are generally poor using lateral flow or ELISA methods. Serologic tests are appealing because they generally are rapid compared to the other methods, require very little material to test, and minimize the chances of infecting those who would otherwise handle infected tissues (e.g. sputum samples) from patients with active disease. Serum tests offer also the possibility of detecting latent inactive disease so that it can be treated early, minimizing future complications and spread from one individual to another. This is of particular interest in HIV TB infected patients who rapidly succumb to TB and can spread the infection to other contacts.

Precisely why it is that circulating mycobacterial antibodies have so elusively escaped detection is unknown. There are many postulates. The exemplary methods, compositions, imaging software and instrument shown in the present disclosure, when used in a disease closely similar to human TB (M. bovis infecting cattle and badger) has with high sensitivity and specificity already demonstrated that antibodies are abundantly present and disease is easily detected in the active and latent forms. M. bovis (Mbv) serologic testing, like TB serologic testing in humans, has historically failed to accurately detect and distinguish infected animals from uninfected animals. (Wood, P. R. and Rothel, J. S. In vitro immunodiagnostic assays for bovine tuberculosis. 1994, Vet. Micro, 40: 125-135.) Using the same methods, compositions and apparatus, a rapid, accurate and cost effective test for human TB can be achieved.

The exemplary embodiments, disclosed herein for Mycobacterium bovis (Mbv) infection provide an ideal serological method for widespread screening of TB infection in cattle. It has also been shown to work well in badger testing. The test appears to yield highly accurate results in less than 2 hours for an initial screen, and does not require a high level of technical expertise. Optimizing on both sensitivity and specificity for minimal false positives is done using reflex supplemental testing of sera. The same method could be used to detect TB in humans with high specificity and sensitivity with just a single drop of blood.

Badger TB Studies

A collaborative study was performed with Mark Chambers (VLA) to test badger serum for TB. The study demonstrated that it is possible to differentiate badgers confirmed by culture for Mbv as either positive or negative for Mbv antibody. Mbv culture positive badgers produced antibodies to various epitopes including MPB83 and CFP10_ESAT6 (fusion protein).

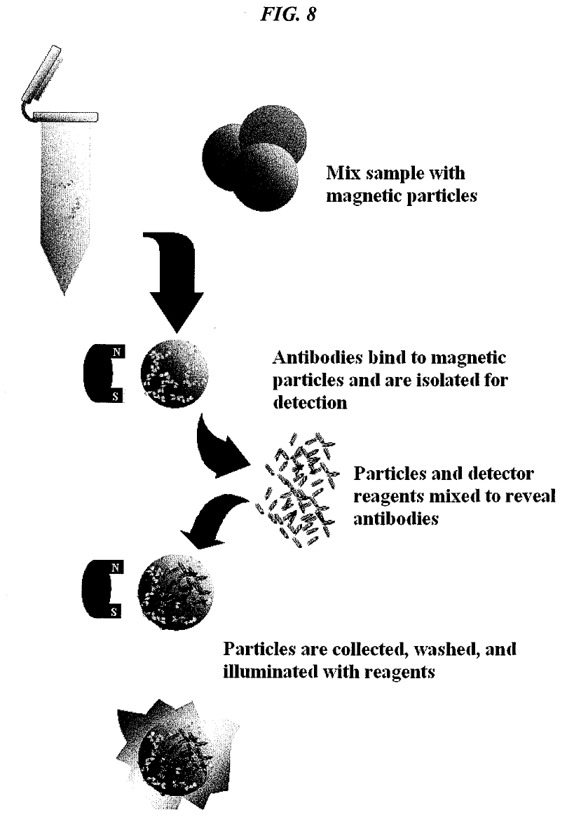

An antibody based assay was used to identify non-infected and positively infected badgers. Optimization results were obtained and reported based on sample badger sera from H&E and culture confirmed Mbv positive and non-infected (negative) badgers. Negative control studies and reference values were obtained by studying known captive, never infected badgers. The general scheme for detecting antibodies against mycobacterial antigens is as shown in FIG. 8.

In a set of 600 samples analyzed in a blinded format, it was shown that the sensitivity and specificity was comparable to VLA gamma interferon testing and the Rapid Diagnostic assay was certainly not as costly or complex to run. The SeraLyte-Mbv™ assay method is currently in use at the VLA Langford site in a badger vaccine testing program.

In an experiment using BCG-vaccinated badgers never exposed to Mbv, the SeraLyte-Mbv™ assay did not cross-react with BCG. When the vaccinated badgers were artificially infected with Mbv and proven by culture to have the disease, the SeraLyte-Mbv™ detected the infected animals. These results demonstrate that there is no interference by BCG in the SeraLyte-Mbv™.

The statistics for 1146 badger samples assayed by simple screening and by Reflex Supplemental Testing are shown in Table 6. Nearly 600 of the samples were tested with the operator blinded to sample identity. Because the image acquired in our assay is automatically interpreted, the operator is essentially blinded to each sample processed, having no means to change the interpretation once the automated cassette image is obtained.

TABLE 6
Summary Statistics for 1146 Badger
Sera Positive or Negative for Mbv

| | Samples | Sens | Spec |
|---|---|---|---|
| Screen | | | |
| Screen #1 | 147 | 92 | 92 |
| Screen #2 | 147 | 88 | 90 |
| Screen #3 | 147 | 90 | 98 |
| Screen #4 | 72 | 93 | 94 |
| Screen #5 | 159 | 88 | 93 |
| Screen #6 | 327 | 93 | 94 |
| Screen #7 | 147 | 88 | 93 |
| | 1146 | Total Badger samples tested | |
| | | on Optimized Assay | |
| Reflex Supplemental | | | |
| Tests | | | |
| RST #1 | | 85 | 100 |
| RST #2 | | 85 | 100 |
| RST #3 | | 84 | 100 |
| RST #4 | | 85 | 100 |
| RST #5 | | 83 | 100 |
| RST #6 | | 83 | 100 |
| RST #7 | | 86 | 100 |

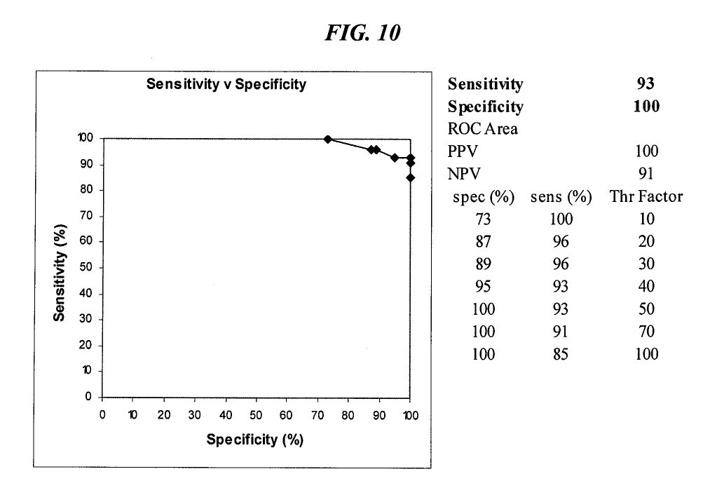

In a set of 306 blinded badger serum field samples, using only a single screening method of analysis SeraLyte-Mbv™ tests scored high in sensitivity and specificity as shown in FIG. 10.

Two epitopes were tested. MPB83 was used to test in all sera in a very large number of badger and cattle provided by investigators in the UK. We also tested again CFP10_ESAT6 in vaccinated and non-vaccinated badger sera that tested positive for MPB83. Assays were first optimized with assignments based on culture, H&E, and post-mortem examinations and subsequently in blinded studies shown to accurately by serum analysis alone strongly correlate with the positive and negative for disease assignments. MPB83 ferrite conjugates appear to accurately identify artificially infected vaccinated captive badger. This was also demonstrated in cattle using appropriate antibody combinations. MPB83 antigens on ferrite probes will differentiate between infected and BCG vaccinated non-infected animals.

Cattle TB Testing

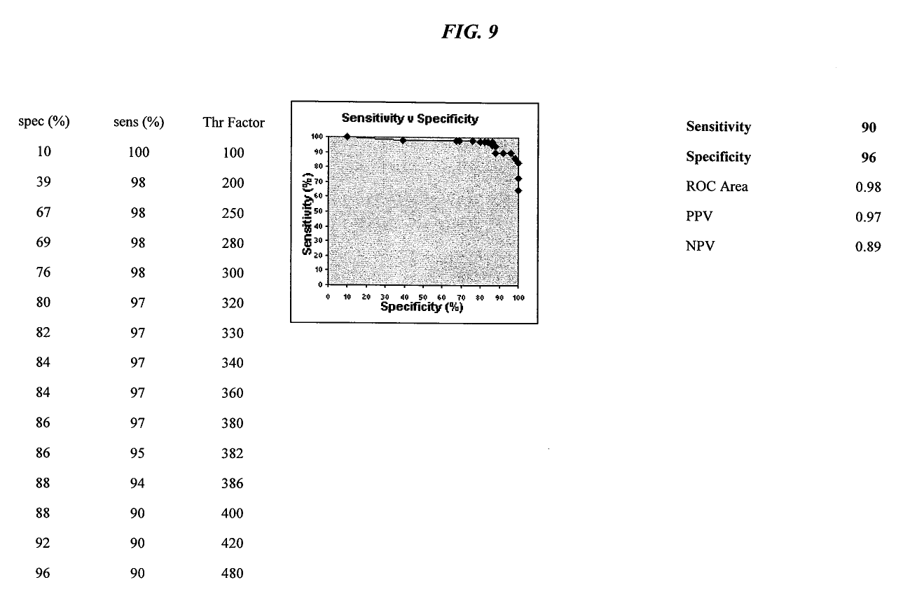

Frozen ampoules of UK tested cattle sera were provided by Martin Vordermeier (UK) with assignments based on skin testing and culture results. Cattle sera were rapidly identified as either positive or negative for infection based on either MPB83 or CFP10_ESAT6 ferrite conjugates. The kind of specificity and sensitivity obtained in testing of cattle is shown in FIG. 9 based on optimization protocols.

TABLE 6
Screening and RSTest Results for Cattle and Badger Infected with Mbv

| | Sensitivity | Specificity | PPV | NPV | ROC |
|---|---|---|---|---|---|
| Screening | | | | | |
| Cattle CFP10_ESAT6 | 94 | 84 | 55 | 99 | 0.94 |
| Cattle MPB83 | 90 | 96 | 97 | 89 | 0.98 |
| Badger MPB83 | 95 | 93 | 86 | 89 | 0.96 |
| Supplemental Reflex | | | | | |
| Cattle CFP10_ESAT6 | 88 | 99 | 94 | 96 | n/a |
| Badger MPB83 | 85 | 100 | 100 | 79 | n/a |

The shape of the ROC graph and statistical values for sensitivity (92%) and specificity (94%) were calculated, see Table 7. Due to the limited supply of serum after optimization, additional data was not collected for RST analysis.

Reflex Supplemental Testing:

No single test will ever consistently produce the level of sensitivity and specificity required to minimize the number of false positives that are expected to occur even in a good assay procedure. Reflex supplemental testing involves testing deliberately at a threshold where false positives are expected and tolerated during the screening part of the assay, because the threshold for detecting positives has been set sufficiently low to catch 100% of positively infected animals. This is done to improve upon the statistics, accurately identifying positive reactors. The reflex supplemental testing (RSTest) of all positive samples in the screening iteratively eliminates false positives at the rate characterized by screening specificity. In this way true positive sera are retained and identified whilst false positive samples are in subsequent testing shown to be truly negative for disease. We have found this method of testing most efficient in identifying true positives while minimizing the number of incorrectly assigned positives. With the high specificity and sensitivity in the currently disclosed assay, relatively few repetitions are required for ideal test results. The same procedure could be applied in testing for TB in humans.

More than 1500 cattle tests have been done, with many improvements in the assay optimization protocols. Proper selection of antibodies and antigen for ferrite conjugation is important. We have come to appreciate too, how strongly certain proteins can interfere in the testing of sera. For example, Protein A even at extraordinarily low levels accompanying the antigen when affixed to the ferrite probe causes so much interference that the tests are nearly useless. Many false positive outcomes are seen without appropriate steps to eliminate these kinds of contaminants. Most antibodies are affinity purified on Protein A columns, introducing one of the significantly interfering components that many investigators have failed to recognize as problematic. This may explain in part why lateral flow and ELISA methods are rather insensitive detectors.

Despite the limited quantity of cattle serum available for testing, we identified the ideal antibody combination for best performance based on the ROC analysis. With many replicates and more than 250 sample sets, including 136 culture confirmed positive and 118 culture confirmed negative serum samples, we successfully differentiated the positive from the negative serum samples.

### Example 5

### Human TB Testing

The MPB83 antigen has been used successfully not only in SeraLyte-Mbv™ assays for screening cattle and badgers for Mbv infection, but also in detecting Mtb (or Mbv) infections in humans, demonstrating the robustness of our platform. Although it was initially believed that a single epitope would not be effective in screening for TB infection in multiple species, the MPB83 probe has been shown to work extremely well in all three species with high Sensitivity and Specificity.

The SeraLyte-TB™ human assay was optimized using sera from 36 subjects with culture confirmed Mtb and 42 serum samples negative for antibody obtained from a commercial provider of human sera. These optimization studies reveal high specificity and sensitivity (up to 95% and 97%, respectively) at an appropriate dilution of human serum with a well defined ROC graph.

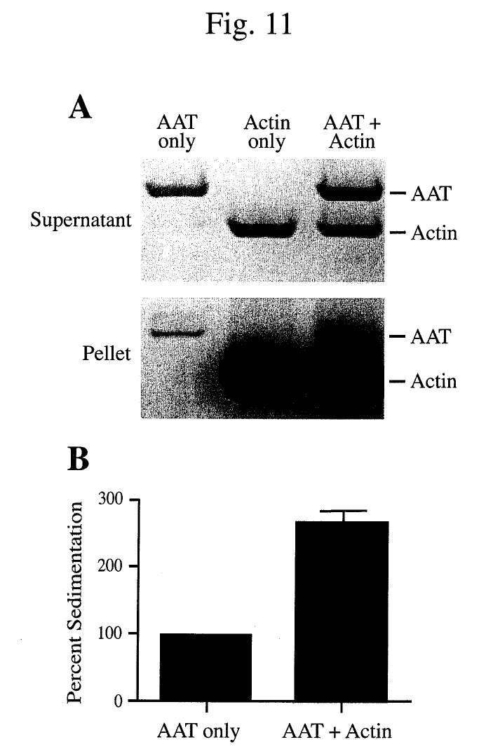

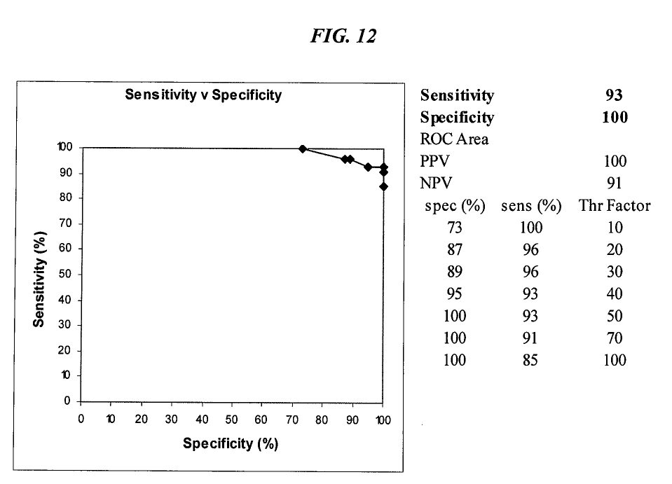

FIG. 11 shows the ROC graph we obtained on the human sample sets with sensitivity and specificity paired to the threshold value used to assign subjects to one or the other of the categories (Positive or Negative for Infection). Multiple replicates, of 36 positives and 42 negatives were tested totaling 253 samples, were used to construct the ROC graph and for calculations of sensitivity and specificity.

As has been observed in cattle and badger testing, the MPB83 ferrite probe appears to accurately differentiate serum containing antibodies against tuberculosis from serum free of antibody against the tuberculosis bacillus.

### Example 6

### USDA Cattle Samples Tested

Over 1,100 cattle serum samples received from Dr. Ray Waters and Steven Hennager of the USDA were tested, of which 444 of these samples proved viable test results. The Kansas De-identified Brucellosis testing samples, were shipped directly from Kansas State University's Veterinary Diagnostic Laboratory.

Over 600 of the 1993 Texas Hillcrest Herd samples were made available for validation testing this year. These had been lyophilized and stored by the USDA for an estimated thirteen years. These samples turned out to be disappointing to all groups that testing them this year, as their poor quality provided no meaningful data.

TABLE 8
Summary of USDA Cattle Samples Tested

| USDA | | | viable |
|---|---|---|---|
| Samples | | Year Samples | for |
| Received | Source | Collected | testing |
| 624 | Texas Hillcrest Herd | Estimated 1993 | no |
| 90 | USDA Artificially Infected cattle | Estimated 2003 | yes |
| | and negative controls | | |
| 57 | Michigan Mbv Reactor Herd | 2006 | yes |
| | Testing | | |
| 10 | Michigan Mbv Reactor Herd | 2006 | yes |
| | Testing | | |
| 87 | Texas Mbv Reactor Herd Testing | 2006 | yes |
| 200 | Kansas State De-identified | 2006 | yes |
| | Brucellosis Testing | | |

Ninety (90) frozen lyophilized samples from a set of artificially infected and negative control animals were evaluated. Even though the samples were approximately three years old, SeraLyte-Mbv™ detected with great accuracy samples that were positive as confirmed by culture. All samples were tested in a blinded format. Four of the cattle were inoculated with M. kansasii and 23 were inoculated with M. avium. The SeraLyte assay showed positive results for the M. kansasii samples and negative results for the M. avium samples, providing good differentiation. The ROC graph and sensitivity (93%) and specificity (100%) for this initially blinded and subsequently unblinded test are shown in FIG. 4.

In another set of USDA serum samples collected from a reactor herd in Michigan, 14 out of 57 of the cattle tested positive in the SeraLyte-Mbv™ test. Michigan State testing results confirmed that 12 of the 14 samples we identified as positive had a positive caudal fold test result. One of the others we identified as positive in the serologic test was positive for both the caudal fold and comparative cervical tuberculin test, and the other sample we identified as positive was also thought to have been positive on both tests as well, but has not yet been confirmed.

We also tested 87 recent samples from a Texas herd of cattle where a positive reactor had been identified, and detected 17 out of the 87 positive by serological testing for Mbv infection. However, in this set of samples it could not be confirmed by the USDA that any were truly infected, raising the concern that some samples were interpreted as false positive by our serologic test. This lead to our request to the USDA to provide some presumed negative samples from within the USDA's various testing programs.

We were able to obtain 200 additional cattle serum samples from the Kansas State University Veterinary Diagnostic Laboratory to help confirm test specificity. This group of cattle was from a low Mbv prevalence area and presumed negative. These samples were de-identified residuals from the laboratory's cattle Brucellosis testing program. All 200 samples tested negative. Based on these findings, it appears highly probable that the cattle from the Texas reactor herd which tested positive by SeraLyte-Mbv are likely positive for Mbv.

### Example 7

### RST and Test Optimization

A question is, how many reflex supplemental tests are needed? Too many tests are of no value because time, money and resources are then wasted. The RST number can be easily calculated based on the specificity, sensitivity, and number of samples that are tested. Both seropositive and seronegative animals will have a test value that can be easily determined in an assay. A normal distribution curve of values for both the infected (positive) and uninfected (negative) animals can be determined.

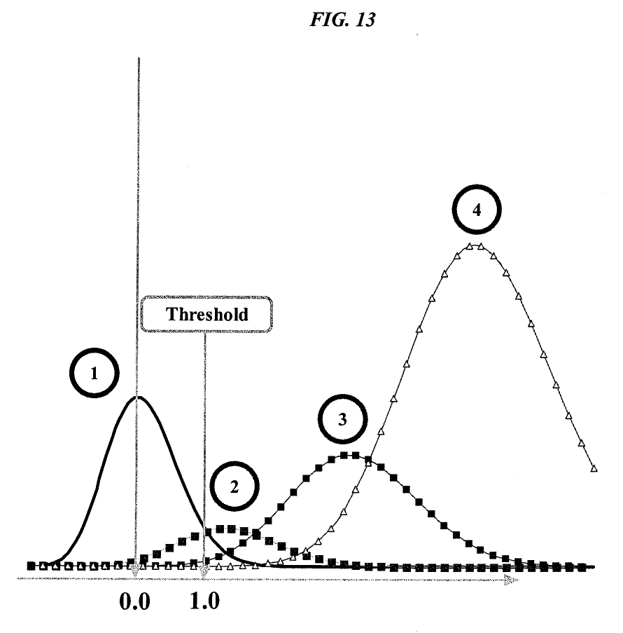

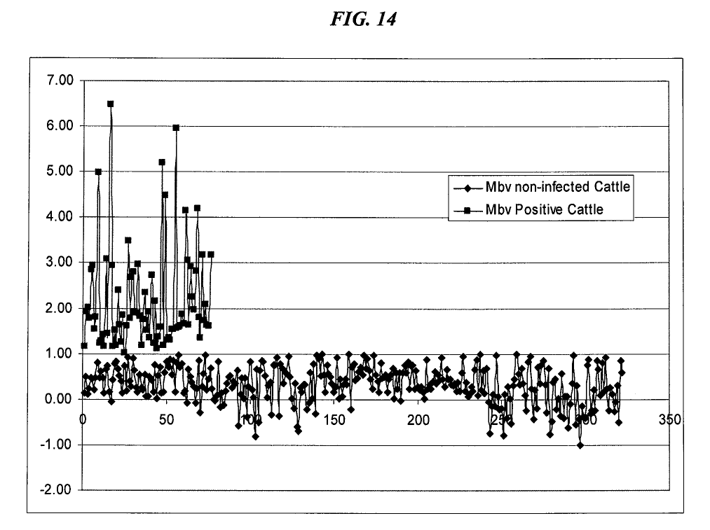

FIG. 1 represents an ideal situation revealing that even when the curves overlap, by using the RST method, the number of positive samples relative to negative false positive samples is increased with each reflex (re-testing positive samples at the same threshold to eliminate false positives with little or no loss in the number of positive samples in the set). However, a more realistic picture as may be experienced in true field testing is illustrated in FIG. 13. The truly negative serum samples should provide a signal virtually identical to a reference negative control sample. Subtracting the reference sample's relative luminosity from the test samples luminosity reveals how different the samples are in signal intensity. For truly negative samples, the value should approximate 0.0 as shown below. However, non-specific interactions result in the serum and other proteins binding to the ferrite particles and they may associate with the reporter HRPSA to make a signal greater then 0.0. This effect results in the broadening at the base of the negative for Mbv serum sample distribution profile making it difficult to differentiate certain negative samples from weakly positive for Mbv samples.

(Test Sample)_{RL}−(Negative Control Reference)_{RL}˜0.0

Because serum protein samples can vary considerably from one animal to the next depending on several factors including nutrition, the presence or absence of other diseases, stresses, etc., a different truly negative for Mbv animal may test above or below 0.0 depending on the specific interactions for that serum sample with the ferrites relative to the manner in which the proteins from the negative control sample interacts. It is therefore important to reduce to the highest degree possible all non-specific interactions for negative serum samples to better differentiate a positive from a negative for Mbv serum sample.

Assay Optimization

Ferrite particles nonspecifically bind to many proteins and are not specific for the target antibody even with a conjugated antigen because of the variety of large folded protein hydrophobic and hydrophilic regions. These interactions trapping non target antibodies intensify the signal for a true negative leading to the possibility of a false positive outcome, and making it more difficult to tell a weakly positive from a true negative sample non specifically interacting with reagents.

Optimizing the assay requires certain knowledge of true positive and true negative samples that can be tested under various conditions to see if relative luminosity differences accurately identified positive for Mbv serum samples. Since, in the early stage of optimization little is known about the ideal conditions for making a difference in signal, robust positive samples are used against known negative samples. As the assay proves to adequately differentiate samples, and more samples are tested, including positive samples with near threshold values for the cut off, the high sensitivity and specificity experienced with certain robust samples may not be observed unless other measures are used.

Therefore, it is necessary to devise a strategy that will sustain high sensitivity and specificity in the face of varied and unknown new sample sets that are to be tested, some of which may be very low tittered (near threshold) sample sets. It is not always appreciated that the distribution of positive and negative sample signals can vary considerably as noted above. For example under FIG. 13, curve 2 early in the infection with an immune incompetent sample set, the ability to differentiate positive from negative samples is difficult because such weak signals and low levels of antibody exist. If the prevalence is also low, this becomes an almost impossible task.

It should be appreciated that if an assay is working, and the results are used to eliminate positively infected animals rendering an over all reduction in prevalence, the test itself is more rigorously challenged in subsequent testing. This is true because with fewer and fewer positive animals in a herd, the more borderline cases are hard to detect. The threshold for detection should be properly selected. It should be below the level where a positive sample would be expected, to capture with 100% sensitivity, but sufficiently high that reflex testing will eliminate false positive results.

As antibody levels increase, it becomes easier to tell a positive from a negative sample. Since we can never predict in advance the prevalence, or status of the animal's immune response, we devised a strategy to optimize accurate detection for all unknown sample sets. As can be seen, by setting the threshold to the 1.0 level, where virtually all of the positive for Mbv animals are detected (except for immune deficient animals), we accept false positives in the first screen. We then test again, only those positives out of the screen to eliminate false positives iteratively as previously described.

Practical issues govern how many reflex tests are ideal because it is possible that in the set of samples being tested, the distribution is quite different then the set that was used during optimization, or for any other testing that had been done with the assay.

Reflex testing will certainly eliminate false positives, but for subsets of cattle testing near the threshold because they are either immune suppressed, malnourished, or otherwise impaired, the signal will be possibly lost randomly with each repeat where the signal is at the cut off point (FIG. 13, 1.0). Some true positive samples might be missed and incorrectly assigned a negative value if restrictions on RST are not in place. Since we can not generally know in advance the animal's antibody status, it is only safe to run a single reflex providing specificity is high for truly negative serum sets. In this way false negatives are also minimized.

If the specificity is ˜90%, in the first screen we have reduced the population of negatives to 10%, and in the single reflex to 1% of the samples testing positive. That is to say, only 1 in 100 of the samples testing positive is a false positive. So we have in a single reflex increased specificity to 99% with expected little loss in true positives unless we are dealing with seriously immune incompetent animals. Even with a specificity of 80%, the single reflex results in an expected specificity of 96%.

We can test truly negative samples. So it is important to show specificity >80% at the threshold where virtually all of the positive samples are detected and differentiated from the negative sample sets. That observation leads to the realization that if we are to test fewer then 30 samples in a set, with the hopes of detecting a single positive sample in the set, reflex testing will not likely improve our results since 2 out of 30 of the samples would test false positive, and reflex testing could result in the loss of a true positive if it happened to be near the threshold for detection.

These findings have led us to the following protocol for reflex supplemental testing in cattle, and should hold true for any other assay as well.
If the sample set for testing consists of 30 or fewer true negatives, do not proceed to the RST.
If the sample set for testing consists of more than 30 true negatives, do proceed to a single reflex for optimal detection.

For example, if we have a set of 40 samples to test, but know the prevalence is ˜25%, we would not run a reflex test, because there are anticipated to be only 30 true negatives in that set. So we also observe generally, that foreknowledge of prevalence will be helpful in planning how to most accurately differentiate positive from negative for Mbv animals. If in the example of 40 samples, 35 were seen to be negative in the screening protocol, we would then realize that a reflex test was warranted for optimal detection. We adjust our software to take these matters into consideration so that a technician involved in testing will be properly advised how to proceed for optimal detection.

Serum samples (n=200 cattle) were diluted and tested in a single screen and subsequent reflex test for cattle, differentiating Mbv infected animals from non-infected animals (Mexican Herd Test April 2007). Two serum samples were taken from each animal, one before and the other after a protein challenge injection. Although no evidence was seen indicating the protein challenge had an effect on the test status, 12 of the cattle were so close to the threshold that they could only be classified as indeterminate. Twenty five (25) cattle tested positive for Mbv before and after challenge, and 147 tested negative for Mbv before and after the protein challenge. The indeterminate in this example are most probably either malnourished, immune suppressed, or under stress affecting antibody production.

Minimizing Nonspecific Protein Binding to Ferrites:

We have noted the importance of minimizing nonspecific interactions with ferrites. Blocking with BSA or other proteins does not always adequately eliminate non-specific interaction because the protein interactions are reversible. A serum protein may more avidly compete for binding sites rendering a falsely positive signal.

Covalent binding of BSA first to the ferrite and then BSA to the antigen (i.e. MPB83) works well as has been demonstrated. This is true for cattle and badger serum analysis, but has limitations in human testing for TB. Humans consume many varied proteins including cattle proteins, unlike badger and cattle that normally would not consume proteins derived from cattle that would be expected to stimulate an antibody response.

In testing of humans, we found a subset of the population that may be as large as 20% in certain situations that show clearly evidence of anti-BSA antibodies. This could even be observed in infants given cow's milk. Because it is impossible to predict in a sample set if any or all of the samples are contaminated with anti BSA antibodies, we had to find an alternative blocking strategy.

Part of the optimization process for lower limit detection involves selecting a protein to use as a blocking agent that is unlikely to have been presented to the human diet, is large enough to effectively interfere in nonspecific interactions, and can be used as a linker for conjugating antigens to the protein coated ferrite.

We identified and tested initially Glucose oxidase (GO). It is relatively inexpensive, abundant, and easily purified. It also has numerous functional groups that can be used for conjugation to ferrites and antigen alike. When we tested human serum samples that had reacted with BSA coated ferrites on GO ferrites, most of the false positives were eliminated.

We also tested simultaneously a synthetic polymer, polyvinyl amine, to if we could take advantage of the numerous primary amines to link first to the ferrite, and then through a sulfhydrylization scheme to MPB83. This proved to be very successful and simple. It was also inexpensive and abundant. Testing in cattle and in human samples that had reacted with BSA coated ferrites proved that non-specific interactions in both cattle and humans were dramatically reduced. Moreover, the intensity for a positive cattle sample was much enhanced. Because the synthetic steps are high yielding and straight forward and apparently very reproducible, we now employ vinyl amine coated ferrites with antigen conjugates (MPB83) as the ideal probe for detecting Mbv. We believe it will also prove well in human testing too.

Antigen Selection for Optimal TB Detection:

There are numerous antigens that have been identified as possible candidates for trapping host antibody against the organism. Based on our experience, it appears that a single antigen, MPB83 will adequately detect and differentiate negative from positive serum samples. Frequently it is suggested by others that no single antigen will effectively identify infected animals or individuals serologically because the antibodies are not sufficiently high enough to be detected.

It appears that surface antigens are indeed the most likely first choice in our experience because they are the first to encounter the host's immune system. We do not find evidence that the antibodies rapidly subside once stimulated, and with the more sensitive lower limits of detection we are able to accomplish through optimization procedures, imaging methods, and software early and apparent sustained responses in positively infected animals. As has already been noted, these responses occur as early as 10 days after artificial infection in certain subsets.

All of the COMPOSITIONS, METHODS and APPARATUS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS, METHODS and APPARATUS and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method for detecting a pathogen in a group of samples comprising:
a) assaying the samples for the presence of the pathogen at a sensitivity of about 100%;
b) using the same assay to iteratively retest only those samples that show positive test results; and
c) repeating the iterative retesting on only those samples that show positive test results after each round of testing, until a selective level of accuracy is obtained.

2. The method of claim 1, wherein the iterative retesting is repeated for three, four, five or six cycles.

3. The method of claim 1, wherein the assay has a sensitivity of 100% and a selectivity of 70%.

4. The method of claim 1, wherein the assay has a sensitivity of 99%, 99.5%, 99.8%, 99.9% or 100%.

5. The method of claim 1, wherein the pathogen is a species of Mycobacterium.

6. The method of claim 5, wherein the pathogen is Mycobacterium bovis or Mycobacterium tuberculosis.

7. The method of claim 5, wherein the assay comprises exposing a CP10_ESAT fusion protein or an MPB83 protein or peptide to a sample of blood, serum or plasma from a subject and detecting antibody binding to the fusion protein.

8. The method of claim 7, wherein the subject is a cow, a badger, an elk, a bison, a deer or a human.

9. The reflex supplemental testing method of claim 1, wherein the number of false negative results is zero.

10. The method of claim 7, further comprising detecting the presence of antibodies against CP10_ESAT or MPB83 in a sample using biotinylated goat IgG anti-bovine IgM antibody, horseradish peroxidase conjugated streptavidin and a luminal peroxide solution to generate chemiluminescence.

11. The method of claim 10, wherein chemiluminescence is measured using a Total Optical Assay Device.

12. The method of claim 10, wherein chemiluminescence is measured using a cassette comprising reflective honeycomb cells or a microtiter well plate.

13. The method of claim 10, further comprising performing data analysis on the measured chemiluminescent signal from each sample.

14. The method of claim 13, wherein the data analysis comprises an auto-threshold correction.

15. The method of claim 13, wherein the data analysis comprises a background determination for groups of pixels, the background for the group of pixels set to equal the highest background emission for any pixel in the group.

16. The method of claim 13, wherein the data analysis comprises applying a quantum efficiency correction factor.

17. The method of claim 14, wherein the threshold for a positive result is set at about 1.6 standard deviations above the average test value for multiple negative samples.

18. The method of claim 7, wherein the CP10_ESAT or MPB83 is conjugated to magnetic beads.

19. The method of claim 18, further comprising using spherical magnets to collect the magnetic beads from solution.

20. A method for detecting tuberculosis in a subject comprising:
a) obtaining a sample of blood or plasma from the subject;
b) exposing the sample to ferrite-conjugated MPB83 protein or peptide under conditions allowing binding of anti-MPB83 antibodies in the sample to the ferrite-conjugated protein or peptide;
c) collecting the ferrite bound to the MPB83 protein or peptide and antibodies; and
d) detecting the presence of antibodies attached to the ferrite.

21. The method of claim 20, wherein the presence of antibodies against MPB83 is indicative of the presence of tuberculosis in the subject.

22. The method of claim 20, wherein the ferrite particles are collected using spherical neodymium magnets.

23. The method of claim 20, wherein the antibodies are detecting using a biotinylated second are antibody.

24. The method of claim 23, further comprising adding streptavidin-conjugated horseradish peroxidase, luminol and peroxide.

25. The method of claim 24, further comprising measuring the light emission produced by chemiluminescent reaction of luminol and peroxide.

26. The method of claim 25, wherein the light emission occurs in a microtiter well plate, said plate with a transparent bottom and with the sides of said wells silvered to reflect light.

27. The method of claim 26, wherein the microtiter well plate is covered with a reflective cover.

28. The method of claim 27, wherein light emission is measured using a CCD device located below the microtiter well plate.

29. The method of claim 28, wherein the wells of the microtiter plate fit into holes drilled into a machined aluminum block located above the CCD device.


SEQUENCE LISTING (6 sequences)

SEQ ID NO: 1 - protein, 100 aa, Mycobacterium tuberculosis
MAEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 2 - DNA, 303 bp, Mycobacterium tuberculosis
atggcagagatgaagaccgatgccgctaccctcgcgcaggaggcaggtaatttcgagcggatctccggcgacctgaaaacccagatcgaccaggtggagtcgacggcaggttcgttgcagggccagtggcgcggcgcggcggggacggccgcccaggccgcggtggtgcgcttccaagaagcagccaataagcagaagcaggaactcgacgagatctcgacgaatattcgtcaggccggcgtccaatactcgagggccgacgaggagcagcagcaggcgctgtcctcgcaaatgggcttctga

SEQ ID NO: 3 - DNA, 32 bp, Artificial
  Inter-ORF region of probe
cccgctaatacgaaaagaaacggagcaaaaac

SEQ ID NO: 4 - protein, 95 aa, Mycobacterium tuberculosis
MTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 5 - DNA, 288 bp, Mycobacterium tuberculosis
atgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgcatag

SEQ ID NO: 6 - DNA, 624 bp, Artificial
  probe
atggcagagatgaagaccgatgccgctaccctcgcgcaggaggcaggtaatttcgagcggatctccggcgacctgaaaacccagatcgaccaggtggagtcgacggcaggttcgttgcagggccagtggcgcggcgcggcggggacggccgcccaggccgcggtggtgcgcttccaagaagcagccaataagcagaagcaggaactcgacgagatctcgacgaatattcgtcaggccggcgtccaatactcgagggccgacgaggagcagcagcaggcgctgtcctcgcaaatgggcttcggacccgctgaatacgaaaagaaacggagcaaaaacatgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgcatag